(19) United States
(12) Patent Application Publication
(10) Pub. No.: US 2024/0228645 A1
(43) Pub. Date: Jul. 11, 2024

(54) ANTIBODIES TARGETING BAFF-R AND USE THEREOF

(71) Applicant: Dragonfly Therapeutics, Inc., Waltham, MA (US)

(72) Inventors: Benjamin FISCHER, Waltham, MA (US); Pyae P. HEIN, Malden, MA (US); Alexander IVANOV, Waltham, MA (US); Xinbi LI, Waltham, MA (US); Matthew SCHNEIDER, Natick, MA (US)

(21) Appl. No.: 18/620,375

(22) Filed: Mar. 28, 2024

Related U.S. Application Data
(63) Continuation of application No. PCT/US2022/077068, filed on Sep. 27, 2022.
(60) Provisional application No. 63/250,092, filed on Sep. 29, 2021.

(51) Int. Cl. C07K 16/28; A61K 39/00; A61K 47/68; C07K 14/525; C07K 14/54; C07K 14/55; C07K 14/56; C12N 5/0783

(52) U.S. Cl. CPC C07K 16/2878; A61K 39/4611; A61K 39/4613; A61K 39/4631; A61K 39/4633; A61K 39/4643; A61K 47/68031; A61K 47/68033; A61K 47/68035; A61K 47/68037; A61K 47/6807; A61K 47/6849; C07K 14/525; C07K 14/5406; C07K 14/5428; C07K 14/5434; C07K 14/5443; C07K 14/55; C07K 14/56; C07K 16/2809; C12N 5/0636; C12N 5/0646; C07K 2317/31; C07K 2317/92; C07K 2319/02; C07K 2319/03; C12N 2510/00

(57) ABSTRACT

Disclosed are proteins with antibody heavy chain and light chain variable domains that can be paired to form an antigen-binding site targeting BAFF-R on a cell, pharmaceutical compositions comprising such proteins, and therapeutic methods using such proteins and pharmaceutical compositions, including for the treatment of cancer or autoimmune disease.

### CROSS REFERENCE

This application is a continuation of International Patent Application No. PCT/US2022/077068, filed Sep27, 2022, which claims the benefit of US Provisional Application No. 63/250,092, filed Sep29, 2021. The entire contents of each of the foregoing applications are incorporated herein by reference in their entirety.

### SEQUENCE LISTING

This application contains a computer readable Sequence Listing which has been submitted in XML file format via Patent Center, the entire content of which is incorporated by reference herein in its entirety. The Sequence Listing XML file submitted via Patent Center is entitled “14247-699-999_SequenceListing.xml,” was created on Mar. 27, 2024, and is 114,456 bytes in size.

### FIELD OF THE INVENTION

The invention provides proteins with antibody heavy chain and light chain variable domains that can be paired to form an antigen-binding site targeting BAFF-R on a cell, pharmaceutical compositions comprising such proteins, and therapeutic methods using such proteins and pharmaceutical compositions, including for the treatment of cancer or autoimmune disease.

### BACKGROUND

Cancer continues to be a significant health problem despite the substantial research efforts and scientific advances reported in the literature for treating this disease. Some of the most frequently diagnosed cancers in adults include prostate cancer, breast cancer, and lung cancer. Hematological malignancies, though less frequent than solid cancers, have low survival rates. Current treatment options for these cancers are not effective for all patients and/or can have substantial adverse side effects. Other types of cancer also remain challenging to treat using existing therapeutic options.

BAFF-R, also called BAFF receptor, TNF receptor superfamily member 13C (TNFRSF13C), CD268, or BR3, is a type III transmembrane protein of the TNF receptor superfamily. BAFF-R is expressed at the late transitional (T2) B-cell stage and on all mature B cells, is downregulated on germinal center B cells, is re-expressed on memory cells, and is absent on plasma cells (Davidson (2012) Curr. Rheumatol. Rep., 14(4): 295-302). BAFF-R is a receptor for B cell-activating factor (BAFF), a B cell survival factor. BAFF can engage three receptors: BAFF-R, transmembrane activator and CAML interactor (TACI), and B-cell maturation antigen (BCMA). Among these three receptors, BAFF-R is the principal receptor involved in the development of follicular and marginal zone splenic B cells (Schiemann et al. (2001) Science, 293: 2111-14).

The BAFF/BAFF-R signaling axis may play a role in B cell hyperplasia. Increased expression of BAFF-R, as well as elevated serum levels of BAFF, has been observed in non-Hodgkin lymphoma (NHL) patients (Shen et al. (2016) Adv. Clin. Exp. Med., 25(5):837-44). Certain single nucleotide polymorphisms (SNPs) in BAFF-R are associated with increased risk of chronic lymphocytic leukemia (CLL) (Jesek et al. (2016) Tumour Biol., 37(10):13617-26). The BAFF/BAFF-R axis is also implicated in autoimmune inflammatory diseases (Mackay et al. (1999) J. Exp. Med., 190:1697-1710). Some systemic lupus erythematosus (SLE) patients have increased levels of BAFF in serum (Cheema et al. (2001) Arthritis Rheum., 44:1313-19), and BAFF-R is consistently occupied on blood B cells in SLE (Carter et al. (2005) Arthritis Rheum., 52:3943-54). Given the observation that autoreactive B cells have a greater dependency on BAFF for their survival as compared with protective B cells (Lesley et al. (2004) Immunity, 20:441-53), it has been proposed that abnormally high levels of BAFF may contribute to the pathogenesis of autoimmune diseases by enhancing the survival of autoreactive B cells.

Accordingly, there remains a need in the field for new and useful antibodies that bind BAFF-R, particularly antibodies that bind to BAFF-R and inhibit its interaction with BAFF.

### SUMMARY OF THE INVENTION

The present invention provides antigen-binding sites that bind BAFF-R. Proteins and protein conjugates containing such antigen-binding sites, for example, antibodies, antibody-drug conjugates, bispecific T-cell engagers (BiTEs), and immunocytokines, as well as immune effector cells (e.g., T cells) expressing a protein containing such an antigen-binding site (e.g., a chimeric antigen receptor (CAR)), are useful for treating BAFF-R-associated diseases such as cancer and autoimmune disease.

Accordingly, in one aspect, the present invention provides an antigen-binding site that binds or is capable of binding BAFF-R, comprising:
a heavy chain variable domain (VH) comprising a complementarity-determining region 1 (CDR1) sequence comprising an amino acid sequence of SEQ ID NO:50, a complementarity-determining region 2 (CDR2) sequence comprising an amino acid sequence of SEQ ID NO:51, and a complementarity-determining region 3 (CDR3) sequence comprising an amino acid sequence of SEQ ID NO:52; and
light chain variable domain (VL) comprising a CDR1 sequence comprising an amino acid sequence of SEQ ID NO:4, a CDR2 sequence comprising an amino acid sequence of SEQ ID NO:5, and a CDR3 sequence comprising an amino acid sequence of SEQ ID NO:49.

In another aspect, provided herein is an antigen-binding site that binds or is capable of binding BAFF-R, wherein:
(a) the VH comprises CDR1, CDR2, and CDR3 sequences identical to the amino acid sequences of SEQ ID NOs: 46, 47, and 48, respectively; and the VL comprises CDR1, CDR2, and CDR3 sequences identical to the amino acid sequences of SEQ ID NOs: 4, 5, and 49, respectively;
(b) the VH comprises CDR1, CDR2, and CDR3 sequences identical to the amino acid sequences of SEQ ID NOs: 1, 2, and 16, respectively; and the VL comprises sequences identical to the amino acid sequences of SEQ ID NOs: 4, 5, and 6, respectively;
(c) the VH comprises CDR1, CDR2, and CDR3 sequences identical to the amino acid sequences of SEQ ID NOs: 21, 2, and 22, respectively; and the VL comprises CDR1, CDR2, and CDR3 sequences identical to the amino acid sequences of SEQ ID NOs: 4, 5, and 6, respectively;
(d) the VH comprises CDR1, CDR2, and CDR3 sequences identical to the amino acid sequences of SEQ ID NOs: 20, 23, and 26, respectively; and the VL comprises CDR1, CDR2, and CDR3 sequences identical to the amino acid sequences of SEQ ID NOs: : 4, 5, and 6, respectively; or
(c) the VH comprises CDR1, CDR2, and CDR3 sequences identical to the amino acid sequences of SEQ ID NOs: 35, 36, and 37, respectively; and the VL comprises CDR1, CDR2, and CDR3 sequences identical to the amino acid sequences of SEQ ID NOs: 4, 5, and 49, respectively.

In some embodiments, an antigen-binding site comprises a VH comprising CDR1, CDR2, and CDR3 sequences identical to the amino acid sequences of SEQ ID NOs: 46, 47, and 48, respectively; and a VL comprising CDR1, CDR2, and CDR3 sequences identical to the amino acid sequences of SEQ ID NOs: 4, 5, and 49, respectively.

In some embodiments, an antigen-binding site comprises a VH comprising CDR1, CDR2, and CDR3 sequences identical to the amino acid sequences of SEQ ID NOs: 1, 23, and 38, respectively; and a VL comprising CDR1, CDR2, and CDR3 sequences identical to the amino acid sequences of SEQ ID NOs: 4, 5, and 39, respectively.

In some embodiments, an antigen-binding site comprises a VH comprising an amino acid sequence at least 90%, at least 91%, at least 92%, at least 93%, at least 94%, at least 95%, at least 96%, at least 97%, at least 98%, or at least 99% identical to SEQ ID NO:40. In some embodiments, an antigen-binding site comprises a VH comprising a G44C substitution relative to SEQ ID NO:40. In some embodiments, an antigen-binding site comprises a VH comprising the amino acid sequence of SEQ ID NO:40. In some embodiments, an antigen-binding site comprises a VH comprising the amino acid sequence of SEQ ID NO:42.

In some embodiments, an antigen-binding site comprises a VL comprising an amino acid sequence at least 90%, at least 91%, at least 92%, at least 93%, at least 94%, at least 95%, at least 96%, at least 97%, at least 98%, or at least 99% identical to SEQ ID NO:41. In some embodiments, the VL comprises a G100C substitution relative to SEQ ID NO:41. In some embodiments, the VL comprises the amino acid sequence of SEQ ID NO:41. In some embodiments, an antigen-binding site comprises a VL comprising the amino acid sequence of SEQ ID NO:43.

In another aspect, provided herein is an antigen-binding site comprising a VH comprising the amino acid sequence of SEQ ID NO:40 and a VL comprising the amino acid sequence of SEQ ID NO:41, or a VH comprising the amino acid sequence of SEQ ID NO:42 and a VL comprising the amino acid sequence of SEQ ID NO:43. In some embodiments, an antigen-binding site comprises a VH comprising the amino acid sequence of SEQ ID NO:40 and a VL comprising the amino acid sequence of SEQ ID NO:41. In some embodiments, an antigen-binding binding site comprises a VH comprising the amino acid sequence of SEQ ID NO:42 and a VL comprising the amino acid sequence of SEQ ID NO:43.

In some embodiments, the antigen-binding site is present as a single-chain fragment variable (scFv), a Fab fragment, or a monoclonal antibody.

In some embodiments, the antigen-binding site is present as a single-chain fragment variable (scFv).

In some embodiments, the antigen-binding site is present as an scFv comprising an amino acid sequence at least 90% identical to the sequence of SEQ ID NO:44 or SEQ ID NO:45. In some embodiments, the scFv comprises an amino acid sequence of SEQ ID NO:44 or SEQ ID NO:45. In some embodiments, the scFv comprises the amino acid sequence of SEQ ID NO:44. In some embodiments, the scFv consists of the amino acid sequence of SEQ ID NO:44.

In another aspect, provided herein is an antigen-binding site that competes with the antigen-binding site of any of the above embodiments for binding to BAFF-R.

In some embodiments, the antigen-binding site binds human BAFF-R with a dissociation constant (K_{D}) smaller than or equal to 5 nM, as measured by surface plasmon resonance (SPR).

In some embodiments, the antigen-binding site inhibits (e.g., blocks) binding of BAFF-R to BAFF (e.g., by at least 50%, at least 75%, at least 90%, at least 95% or at least 99% as measured in a competitive binding assay).

In another aspect, provided herein is a protein comprising the antigen-binding site of any one of the above embodiments.

In some embodiments, the protein further comprises an antibody heavy chain constant region. In some embodiments, the antibody heavy chain constant region is a human IgG heavy chain constant region. In some embodiments, the antibody heavy chain constant region is a human IgG1 heavy chain constant region. In some embodiments, each polypeptide chain of the antibody heavy chain constant region comprises an amino acid sequence at least 90% identical to the amino acid sequence of wild-type human IgG1 Fc region.

In some embodiments, at least one polypeptide chain of the antibody heavy chain constant region comprises one or more mutations, relative to the amino acid sequence of wild-type human IgG1 Fc region, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system.

In some embodiments, at least one polypeptide chain of the antibody heavy chain constant region comprises one or more mutations, relative to the amino acid sequence of wild-type human IgG1 Fc region, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y4071, Y407V, K409F, K409W, K409D, T411D, T411E, K439D, and K439E, numbered according to the EU numbering system.

In some embodiments, one polypeptide chain of the antibody heavy chain constant region comprises one or more mutations, relative to the amino acid sequence of wild-type human IgG1 Fc region, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to the amino acid sequence of wild-type human IgG1 Fc region, at one or more positions selected from Q347, Y349, L351, S354, E356, E357, S364, T366, L368, K370, N390, K392, T394, D399, D401, F405, Y407, K409, T411, and K439, numbered according to the EU numbering system.

In some embodiments, one polypeptide chain of the antibody heavy chain constant region comprises K360E and K409W substitutions relative to the amino acid sequence of wild-type human IgG1 Fc region; and the other polypeptide chain of the antibody heavy chain constant region comprises Q347R, D399V and F405T substitutions relative to the amino acid sequence of wild-type human IgG1 Fc region, numbered according to the EU numbering system.

In some embodiments, one polypeptide chain of the antibody heavy chain constant region comprises a Y349C substitution relative to the amino acid sequence of wild-type human IgG1 Fc region; and the other polypeptide chain of the antibody heavy chain constant region comprises an S354C substitution relative to the amino acid sequence of wild-type human IgG1 Fc region, numbered according to the EU numbering system.

In another aspect, provided herein is an antibody-drug conjugate comprising the protein of any one of the above embodiments and a drug moiety. In some embodiments, the drug moiety is selected from the group consisting of auristatin, N-acetyl-y calicheamicin, maytansinoid, pyrrolobenzodiazepine, and SN-38.

In another aspect, provided herein is an immunocytokine comprising the antigen-binding site of any one of the above embodiments and a cytokine. In some embodiments, the cytokine is selected from the group consisting of IL-2, IL-4, IL-10, IL-12, IL-15, TNF, and IFNα.

In another aspect, provided herein is a bispecific T-cell engager comprising the antigen-binding site of any one of the above embodiments and an antigen-binding site that binds CD3.

In another aspect, provided herein is a chimeric antigen receptor (CAR) comprising:
(a) an antigen-binding site of the present invention;
(b) a transmembrane domain; and
(c) an intracellular signaling domain.

In some embodiments, the transmembrane domain is selected from the

transmembrane regions of the alpha, beta or zeta chain of the T-cell receptor, CD28, CD3 epsilon, CD45, CD4, CD5, CD8, CD9, CD16, CD22, BAFF-R, CD37, CD64, CD80, CD86, CD134, CD137, CD152, and CD154.

In some embodiments, the intracellular signaling domain comprises a primary signaling domain comprising a functional signaling domain of CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12. In some embodiments, the intracellular signaling domain further comprises a costimulatory signaling domain comprising a functional signaling domain of a costimulatory receptor. In some embodiments, the costimulatory receptor is selected from the group consisting of OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

In another aspect, provided herein is an isolated nucleic acid encoding the CAR of any one of the above embodiments.

In another aspect, provided herein is an expression vector comprising the isolated nucleic acid of the above aspect.

In another aspect, provided herein is an immune effector cell comprising the nucleic acid or the expression vector of the above aspects.

In another aspect, provided herein is an immune effector cell expressing the CAR of any one of the above aspects. In some embodiments, the immune effector cell is a T cell. In some embodiments, the T cell is a CD8+ T cell, a CD4+ T cell, a γδ T cell, or an NKT cell. In some embodiments, the immune effector cell is an NK cell.

In another aspect, provided herein is a pharmaceutical composition comprising the protein, the antibody-drug conjugate, the immunocytokine, the bispecific T-cell engager, or the immune effector cell of any of the above aspects or embodiments; and a pharmaceutically acceptable carrier.

In another aspect, provided herein is a method of treating cancer, the method comprising administering to a subject in need thereof an effective amount of the protein, the antibody-drug conjugate, the immunocytokine, the bispecific T-cell engager, the immune effector cell, or the pharmaceutical composition of any of the above aspects or embodiments. In some embodiments, the cancer is B-cell non-Hodgkin's lymphoma (B-NHL), chronic lymphocytic leukemia (CLL), mantle cell lymphoma (MCL), follicular lymphoma (FL), diffuse large B-cell lymphoma (DLBCL), marginal zone lymphoma, mucosa-associated lymphoid tissue (MALT) lymphoma, primary mediastinal B-cell lymphoma, and acute lymphocytic leukemia (ALL). In some embodiments, the cancer expresses BAFF-R.

In another aspect, provided herein is a method of treating an autoimmune inflammatory disease, the method comprising administering to a subject in need thereof an effective amount of the protein, the antibody-drug conjugate, the immunocytokine, the bispecific T-cell engager, the immune effector cell, or the pharmaceutical composition of any of the above aspects or embodiments.

In some embodiments, protein, the antibody-drug conjugate, the immunocytokine, or the bispecific T-cell engager of any of the above aspects or embodiments is a purified antigen-binding site, protein, antibody-drug conjugate, immunocytokine, or bispecific T cell engager.

In some embodiments, the protein, the antibody-drug conjugate, the immunocytokine, or the bispecific T-cell engager of any of the above aspects or embodiments is purified by a method selected from the group consisting of: centrifugation, depth filtration, cell lysis, homogenization, freeze-thawing, affinity purification, gel filtration, ion exchange chromatography, hydrophobic interaction exchange chromatography, and mixed-mode chromatography.

These and other aspects and advantages of the invention are illustrated by the following figures, detailed description and claims.

### BRIEF DESCRIPTION OF THE DRAWINGS

The invention can be more completely understood with reference to the following drawings.

FIG. 1 is a graph showing fluorescence outputs from a binding assay showing blocking of BAFF-biotin binding to human BAFF-R expressed on CHO cells by the indicated antibodies.

FIG. 2 is a graph showing fluorescence outputs from a blocking assay of BAFF-biotin binding to BAFF-R expressed on CHO cells by the indicated antibodies.

FIGS. 3A-3D are graphs of fluorescence outputs from binding assays on CHO cells showing binding of indicated antibodies to BAFF-R (FIG. 3A, FIG. 3B) or from a blocking assay of BAFF-biotin binding to BAFF-R by indicated antibodies (FIG. 3C, FIG. 3D).

FIGS. 4A-4E are flow cytometry plots showing binding of AB0369scFv expressed on yeast to no antigen control (FIG. 4A), h-BAFF-R-hFc (FIG. 4B), Irrelevant-hFc (FIG. 4C), hBAFF-R-GST (FIG. 4D), or Irrelevant-GST (FIG. 4E). Vertical axes indicates scFv expression as measured by detection of the Flag epitope tag; horizonal axes indicate binding of biotinylated control of BAFF-R constructs to scFv as measured by detection of streptavidin-PE.

FIGS. 5A and 5B are graphs showing binding of AB0369 or indicated controls to human (FIG. 5A) or cynomolgus monkey (FIG. 5B) BAFF-R.

FIGS. 6A and 6B detail a poly-specificity assay of a multi-specific binding proteins with a BAFF-R binding site derived from AB0369. FIG. 6A is a schematic of the assay. FIG. 6B shows graphs of AB0369 (left panels), trastuzumab negative control (middle panels), or ixekizumab positive control (right panels) in the absence (top panels) or presence (bottom panels) of poly-specificity reagent (PSR).

FIG. 7 is a graph showing a KHYG-1-CD16aV cytotoxicity assay of Ramos cells as induced by a multispecific binding protein with a BAFF-R binding site derived from AB0369.

FIG. 8 is a graph showing fluorescence outputs from a binding assay showing blockage of BAFF-biotin binding to human BAFF-R expressed on CHO cells by AB0369 or indicated.

FIGS. 9A-9D are flow cytometry plots showing binding of hBAFF-R-hFc-His to parental AB0369scFv or clones selected from a library produced by affinity maturation expressed on yeast following successive rounds of selection. FIG. 9A shows binding to parental AB0369scFv; FIG. 9B shows binding to sample from the first round of clone selection; FIG. 9C shows binding to sample from the second round of clone selection; FIG. 9D shows binding to output from the second round of clone selection.

FIGS. 10A-10E are flow cytometry plots showing binding of hBAFF-R-hFc-His to AB0369 and affinity-matured scFv clones expressed on yeast. FIG. 10A shows binding to parental AB0369; FIG. 10B shows binding to AB0605; FIG. 10C shows binding to AB0622; FIG. 10D shows binding to AB0622; and FIG. 10E shows binding to an ianalumab-based scFv.

FIGS. 11A-11C are graphs demonstrating BAFF-R binding and cytotoxicity of multi-specific binding proteins developed from affinity maturation of AB0369. FIG. 11A is a graph showing binding of multi-specific binding proteins with BAFF-R binding sites derived from indicated clones to human BAFF-R expressed on CHO cells. FIG. 11B is a graph showing a KHYG-1-CD16aV cytotoxicity assay of Ramos cells as induced by multi-specific binding proteins with BAFF-R binding sites derived from indicated clones. FIG. 11C is a graph showing a KHYG-1-CD16aV cytotoxicity assay of Ramos cells as induced by multi-specific binding proteins with BAFF-R binding sites derived from AB0622.

FIGS. 12A and 12B detail a poly-specificity assay of multi-specific binding proteins with BAFF-R binding sites derived from AB00605 and AB0606. FIG. 12A is a schematic of the assay. FIG. 12B shows graphs of AB0605 (left panels) or AB0606 (right panels) in the absence (top panels) or presence (bottom panels) of poly-specificity reagent (PSR).

FIGS. 13A-13C are flow cytometry plots showing binding of hBAFF-R-hFc-His to parental AB0369scFv or clones selected from a library produced by affinity maturation and expressed on yeast following successive rounds of selection. FIG. 13A shows binding to parental AB0369scFv; FIG. 13B shows binding to sample from the first round of clone selection; FIG. 13C shows binding to sample from the second round of clone selection.

FIGS. 14A-14E are flow cytometry plots showing binding of hBAFF-R-hFc-His to AB0369 and affinity-matured scFv clones expressed on yeast. FIG. 14A shows binding to parental AB0369; FIG. 14B shows binding to AB0679; FIG. 14C shows binding to AB0681; FIG. 14D shows binding to AB0682; and FIG. 14E shows binding to an ianalumab-based scFv.

FIGS. 15A-15C are graphs demonstrating BAFF-R binding to multi-specific binding proteins developed from affinity maturation of AB0369. FIG. 15A is a graph showing binding of multi-specific binding proteins with BAFF-R binding sites derived from indicated clones to human BAFF-R expressed on CHO cells. FIG. 15B is a graph showing binding of multi-specific binding proteins with BAFF-R binding sites derived from indicated clones to cynomolgus monkey BAFF-R expressed on CHO cells. FIG. 15C is a graph showing fluorescence outputs from a binding assay showing blockage of BAFF-biotin binding to BAFF-R expressed on CHO cells by the indicated antibodies.

FIG. 16 is a graph showing a KHYG-1-CD16aV cytotoxicity assay of BJAB cells as induced by multi-specific binding proteins with BAFF-R binding sites derived from AB0679, AB0568, or Tool-F3′ positive control.

FIGS. 17A-17D are flow cytometry plots showing binding of hBAFF-R-hFc-His to parental AB0369scFv clones selected from a library produced by affinity maturation expressed on yeast following successive rounds of selection. FIG. 17A shows binding to parental AB0369scFv; FIG. 17B shows binding to sample from the first round of clone selection; FIG. 17C shows binding to sample from the second round of clone selection; and FIG. 17D shows binding to sample from the third round of clone selection.

FIGS. 18A-18F are flow cytometry plots showing binding of hBAFF-R-hFc-His to AB0369 and affinity-matured scFv clones expressed on yeast. FIG. 18A shows binding to parental AB0369; FIG. 18B shows binding to AB0682; FIG. 18C shows binding to AB0898; FIG. 18D shows binding to AB0899; FIG. 18E shows binding to AB0900; and FIG. 18F shows binding to an ianalumab-based scFv.

FIG. 19 is a graph showing a KHYG-1-CD16aV cytotoxicity assay of BJAB cells as induced by multi-specific binding proteins with BAFF-R binding sites derived from AB0898, AB0899, or AB0900.

FIG. 20 shows graphs of differential scanning calorimetry (DSC) profiles of AB0898 (top panel), AB0899 (center panel), and AB0900 (bottom panel).

FIG. 21 shows flow cytometry plots of binding of scFv clones expressed on yeast to biotinylated hBAFFR-Fc before (left) and after (right) challenge by incubation with 1 mM non-biotinylated hBAFFR-Fc.

FIG. 22 shows flow cytometry plots of binding of scFv clones expressed on yeast to biotinylated hBAFFR-Fc before (top) and after (bottom) challenge by incubation with 1mM non-biotinylated hBAFFR-Fc. Clones tested are (left-to-right) AB1080, AB1081, AB1084, AB1085, and ianalumab-based scFv.

FIGS. 23A and 23B are graphs showing binding of indicated antibody clones to human (FIG. 23A) or cynomolgus monkey (FIG. 23B) BAFF-R.

FIGS. 24A and 24B detail a poly-specificity assay of a multi-specific binding proteins with a BAFF-R binding site derived from AB1080 or AB1081. FIG. 24A is a schematic of the assay. FIG. 24B shows graphs of AB1080 (left panels), AB1081 (middle-left panels), trastuzumab negative control (middle-right panels), or ixekizumab positive control (right panels) in the absence (top panels) or presence (bottom panels) of poly-specificity reagent (PSR).

FIGS. 25A and 25B show graphs of a KHYG-1-CD16aV cytotoxicity assay of BJAB cells as induced by multi-specific binding proteins with BAFF-R binding sites derived from AB1080 (FIG. 25A) or AB 1085 (FIG. 25B) compared to Tool positive control.

FIG. 26 is a graph showing fluorescence outputs from a binding assay showing blockage of BAFF-biotin binding to human BAFF-R expressed on CHO cells by the indicated antibody clones.

FIG. 27 shows graphs of nano-dual scanning fluorimetry (nanoDSF) analysis of multi-specific binding proteins with BAFF-R binding sites derived from AB1080 (left panel), AB1081 (center-left panel), AB1084 (center-right panel), and AB1085 (right panel).

FIG. 28 shows a graph of hydrophobic interaction chromatography (HIC) analysis of multi-specific binding proteins with BAFF-R binding sites derived from indicated antibodies.

FIG. 29 shows a graph of HIC analysis of AB1612 compared to indicated benchmark biologics.

FIGS. 30A and 30B are graphs showing binding of indicated antibody clones to human (FIG. 30A) or cynomolgus monkey (FIG. 30B) BAFF-R.

### DETAILED DESCRIPTION

The present invention provides antigen-binding sites that bind human BAFF-R. Proteins and protein conjugates containing such antigen-binding sites, for example, antibodies, antibody-drug conjugates, bispecific T-cell engagers (BiTEs), and immunocytokines, as well as immune effector cells (e.g., T cells) expressing a protein containing such an antigen-binding site (e.g., a chimeric antigen receptor (CAR)), are useful for treating BAFF-R-associated diseases such as cancer and autoimmune disease. Various aspects of the present invention are set forth in the sections below; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in or is capable of antigen binding. In human antibodies, the antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide. All the amino acid positions in heavy or light chain variable regions disclosed herein are numbered according to Kabat numbering.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the present invention and their pharmaceutically acceptable acid addition salts.

Exemplary bases include, but are not limited to, alkali metal (e.g., sodium) hydroxides, alkaline carth metal (e.g., magnesium) hydroxides, ammonia, and compounds of formula NW4+, wherein W is C1-4 alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, BAFF-R (also known as BAFF receptor, B-cell activating factor receptor, BR3, TNFRSF13C, tumor necrosis factor receptor superfamily member 13C, TNF receptor superfamily member 13C, CD268, and BLyS receptor 3) refers to the protein of Uniprot Accession No. Q96RJ3 and related isoforms and orthologs.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

Various features and aspects of the invention are discussed in more detail below.

### I. Antigen-Binding Site

In one aspect, the present invention provides an antigen-binding site that binds human BAFF-R. The VH, VL, CDR, and scFv sequences of exemplary antigen-binding sites are listed in Table 1. The CDR sequences are identified according to the Chothia numbering scheme.

TABLE 1
Sequences of Exemplary Antigen-Binding Sites that Bind BAFF-R

| Clone | VH | VL |
|---|---|---|
| 1129_A01 | EVQLVQSGGGVVQPGRSLRLSC | EIVLTQSPSSLSASVGDRVTITCR |
| (AB0369 scFv) | AASGFTFSSYGMHWVRQAPGK | ASQSISSYLNWYQQKPGKAPKLL |
| | CLEWVAVIWYDGSNKYYGDSV | IYAASSLQSGVPSRFSGSGSGTDF |
| | KGRFTISRDNSKNTLYLQMNSL | TLTISSLQPEDFATYYCQQSYSTP |
| | RDEDTAVYYCARRFTMLRGLII | LTFGCGTKVEIK (SEQ ID NO: 63) |
| | EDYGMDVWGQGTTVTVSS | CDR1: RASQSISSYLN (SEQ ID |
| | (SEQ ID NO: 77) | NO: 4) |
| | CDR1: GFTFSSY (SEQ ID NO: 1) | CDR2: AASSLQS (SEQ ID NO: 5) |
| | CDR2: WYDGSN (SEQ ID NO: 2) | CDR3: QQSYSTPLT (SEQ ID |
| | CDR3: RFTMLRGLIIEDYGMDV | NO: 6) |
| | (SEQ ID NO: 3) | |
| 1203_A01 | CDR1: GFTFSSY (SEQ ID NO: 1) | CDR1: RASQSVSSNLA (SEQ ID |
| | CDR2: WYDGSN (SEQ ID NO: 2) | NO: 8) |
| | CDR3: RFTMLRGVFIEDYGMDV | CDR2: GASTRAT (SEQ ID NO: 9) |
| | (SEQ ID NO: 7) | CDR3: QQSYSTPLT (SEQ ID |
| | | NO: 6) |
| 1203_A02 | CDR1: GFTFSTY (SEQ ID NO: 10) | CDR1: RASQSISSYLN (SEQ ID |
| | CDR2: WYDGSN (SEQ ID NO: 2) | NO: 4) |
| | CDR3: RNTMVRGVIIEDYGMDV | CDR2: AASSLQS (SEQ ID NO: 5) |
| | (SEQ ID NO: 11) | CDR3: QQSYSSPLT (SEQ ID |
| | | NO: 12) |
| AB0605scfv | EVQLVQSGGGVVQPGRSLRLSC | EIVLTQSPSSLSASVGDRVTITCR |
| | AASGFTFSSYGMHWVRQAPGK | ASQSISSYLNWYQQKPGKAPKLL |
| | CLEWVAVIWYDGSNKYYGDSV | IYAASSLQSGVPSRFSGSGSGTDF |
| | KGRFTISRDNSKNTLYLQMNSL | TLTISSLQPEDFATYYCQQSYSTP |
| | RDEDTAVYYCARRFTMLRGQYI | LTFGCGTKVEIK (SEQ ID NO: 63) |
| | EDYGMDVWGQGTTVTVSS | CDR1: RASQSISSYLN (SEQ ID |
| | (SEQ ID NO: 64) | NO: 4) |
| | CDR1: GFTFSSY (SEQ ID NO: 1) | CDR2: AASSLQS (SEQ ID NO: 5) |
| | CDR2: WYDGSN (SEQ ID NO: 2) | CDR3: QQSYSTPLT (SEQ ID |
| | CDR3: RFTMLRGQYIEDYGMDV | NO: 6) |
| | (SEQ ID NO: 13) | |
| AB0606scfv | EVQLVQSGGGVVQPGRSLRLSC | EIVLTQSPSSLSASVGDRVTITCR |
| | AASGFTFSSYGMHWVRQAPGK | ASQSISSYLNWYQQKPGKAPKLL |
| | CLEWVAVIWYDGSNKYYGDSV | IYAASSLQSGVPSRFSGSGSGTDF |
| | KGRFTISRDNSKNTLYLQMNSL | TLTISSLQPEDFATYYCQQSYSTP |
| | RDEDTAVYYCARRFTMLRGWY | LTFGCGTKVEIK (SEQ ID NO: 63) |
| | IEDYGMDVWGQGTTVTVSS | CDR1: RASQSISSYLN (SEQ ID |
| | (SEQ ID NO: 65) | NO: 4) |
| | CDR1: GFTFSSY (SEQ ID NO: 1) | CDR2: AASSLQS (SEQ ID NO: 5) |
| | CDR2: WYDGSN (SEQ ID NO: 2) | CDR3: QQSYSTPLT (SEQ ID |
| | CDR3: | NO: 6) |
| | RFTMLRGWYIEDYGMDV (SEQ | |
| | ID NO: 14) | |
| AB0622scfv | EVQLVQSGGGVVQPGRSLRLSC | EIVLTQSPSSLSASVGDRVTITCR |
| | AASGFTFSSYGMHWVRQAPGK | ASQSISSYLNWYQQKPGKAPKLL |
| | CLEWVAVIWYDGSNKYYGDSV | IYAASSLQSGVPSRFSGSGSGTDF |
| | KGRFTISRDNSKNTLYLQMNSL | TLTISSLQPEDFATYYCQQSYSTP |
| | RDEDTAVYYCARRFTMLRGWII | LTFGCGTKVEIK (SEQ ID NO: 63) |
| | EDYGMDVWGQGTTVTVSS | CDR1: RASQSISSYLN (SEQ ID |
| | (SEQ ID NO: 66) | NO: 4) |
| | CDR1: GFTFSSY (SEQ ID NO: 1) | CDR2: AASSLQS (SEQ ID NO: 5) |
| | CDR2: WYDGSN (SEQ ID NO: 2) | CDR3: QQSYSTPLT (SEQ ID |
| | CDR3: RFTMLRGWIIEDYGMDV | NO: 6) |
| | (SEQ ID NO: 15) | |
| Consensus 1 | CDR1: GFTFSSY (SEQ ID NO: 1) | CDR1: RASQSISSYLN (SEQ ID |
| (AB0605, AB0606, | CDR2: WYDGSN (SEQ ID NO: 2) | NO: 4) |
| AB0622) | CDR3: | CDR2: AASSLQS (SEQ ID NO: 5) |
| | RFTMLRGX₁X₂IEDYGMDV | CDR3: QQSYSTPLT (SEQ ID |
| | where X₁ is Q or W, and X₂ is I | NO: 6) |
| | or Y (SEQ ID NO: 16) | |
| AB0679scFv | EVQLVQSGGGVVQPGRSLRLSC | EIVLTQSPSSLSASVGDRVTITCR |
| | AASGFPFSSYGMHWVRQAPGK | ASQSISSYLNWYQQKPGKAPKLL |
| | CLEWVAVIWYDGSNKYYGDSV | IYAASSLQSGVPSRFSGSGSGTDF |
| | KGRFTISRDNSKNTLYLQMNSL | TLTISSLQPEDFATYYCQQSYSTP |
| | RDEDTAVYYCARRFTMLRGWY | LTFGCGTKVEIK (SEQ ID NO: 63) |
| | IEDYGMDVWGQGTTVTVSS | CDR1: RASQSISSYLN (SEQ ID |
| | (SEQ ID NO: 67) | NO: 4) |
| | CDR1: GFPFSSY (SEQ ID NO: 17) | CDR2: AASSLQS (SEQ ID NO: 5) |
| | CDR2: WYDGSN (SEQ ID NO: 2) | CDR3: QQSYSTPLT (SEQ ID |
| | CDR3: | NO: 6) |
| | RFTMLRGWYIEDYGMDV (SEQ | |
| | ID NO: 14) | |
| AB0681scFv | EVQLVQSGGGVVQPGRSLRLSC | EIVLTQSPSSLSASVGDRVTITCR |
| | AASGEWFSSYGMHWVRQAPGK | ASQSISSYLNWYQQKPGKAPKLL |
| | CLEWVAVIWYDGSNKYYGDSV | IYAASSLQSGVPSRFSGSGSGTDF |
| | KGRFTISRDNSKNTLYLQMNSL | TLTISSLQPEDFATYYCQQSYSTP |
| | RDEDTAVYYCARRFTHLRGWII | LTFGCGTKVEIK (SEQ ID NO: 63) |
| | EDYGMDVWGQGTTVTVSS | CDR1: RASQSISSYLN (SEQ ID |
| | (SEQ ID NO: 68) | NO: 4) |
| | CDR1: GEWFSSY (SEQ ID NO: 18) | CDR2: AASSLQS (SEQ ID NO: 5) |
| | CDR2: WYDGSN (SEQ ID NO: 2) | CDR3: QQSYSTPLT (SEQ ID |
| | CDR3: RFTHLRGWIIEDYGMDV | NO: 6) |
| | (SEQ ID NO: 19) | |
| AB0682scFv | EVQLVQSGGGVVQPGRSLRLSC | EIVLTQSPSSLSASVGDRVTITCR |
| | AASGFTFSSSGMHWVRQAPGK | ASQSISSYLNWYQQKPGKAPKLL |
| | CLEWVAVIWYDGSNKYYGDSV | IYAASSLQSGVPSRFSGSGSGTDF |
| | KGRFTISRDNSKNTLYLQMNSL | TLTISSLQPEDFATYYCQQSYSTP |
| | RDEDTAVYYCARRFTMLRGWY | LTFGCGTKVEIK (SEQ ID NO: 63) |
| | IEDYGMDVWGQGTTVTVSS | CDR1: RASQSISSYLN (SEQ ID |
| | (SEQ ID NO: 69) | NO: 4) |
| | CDR1: GFTFSSS (SEQ ID NO: 20) | CDR2: AASSLQS (SEQ ID NO: 5) |
| | CDR2: WYDGSN (SEQ ID NO: 2) | CDR3: QQSYSTPLT (SEQ ID |
| | CDR3: | NO: 6) |
| | RFTMLRGWYIEDYGMDV (SEQ | |
| | ID NO: 14) | |
| Consensus 2 | CDR1: GX₁X₂FSSX₃ where X₁ is F | CDR1: RASQSISSYLN (SEQ ID |
| (AB0679, AB0681, | or E, X₂ T, P, or W, X₃ is Y or S | NO: 4) |
| AB0682) | (SEQ ID NO: 21) | CDR2: AASSLQS (SEQ ID NO: 5) |
| | CDR2: WYDGSN (SEQ ID NO: 2) | CDR3: QQSYSTPLT (SEQ ID |
| | CDR3: | NO: 6) |
| | RFTX₁LRGWX₂IEDYGMDV | |
| | where X₁ is M or H, X₂ is I or Y | |
| | (SEQ ID NO: 22) | |
| AB0898 | EVQLVQSGGGVVQPGRSLRLSC | EIVLTQSPSSLSASVGDRVTITCR |
| | AASGFTFSSSGMHWVRQAPGK | ASQSISSYLNWYQQKPGKAPKLL |
| | CLEWVAVIWYDASNKYYGDSV | IYAASSLQSGVPSRFSGSGSGTDF |
| | KGRFTISRDNSKNTLYLQMNSL | TLTISSLQPEDFATYYCQQSYSTP |
| | RDEDTAVYYCARRFTRLRGWYI | LTFGCGTKVEIK (SEQ ID NO: 63) |
| | EDYGLDVWGQGTTVTVSS (SEQ | CDR1: RASQSISSYLN (SEQ ID |
| | ID NO: 70) | NO: 4) |
| | CDR1: GFTFSSS (SEQ ID NO: 20) | CDR2: AASSLQS (SEQ ID NO: 5) |
| | CDR2: WYDASN (SEQ ID NO: 23) | CDR3: QQSYSTPLT (SEQ ID |
| | CDR3: RFTRLRGWYIEDYGLDV | NO: 6) |
| | (SEQ ID NO: 32) | |
| AB0899 | EVQLVQSGGGVVQPGRSLRLSC | EIVLTQSPSSLSASVGDRVTITCR |
| | AASGFTFSSSGMHWVRQAPGK | ASQSISSYLNWYQQKPGKAPKLL |
| | CLEWVAVIWYDASNKYYGDSV | IYAASSLQSGVPSRFSGSGSGTDF |
| | KGRFTISRDNSKNTLYLQMNSL | TLTISSLQPEDFATYYCQQSYSTP |
| | RDEDTAVYYCARRFTYLRGWYI | LTFGCGTKVEIK (SEQ ID NO: 63) |
| | EDYGLDVWGQGTTVTVSS (SEQ | CDR1: RASQSISSYLN (SEQ ID |
| | ID NO: 71) | NO: 4) |
| | CDR1: GFTFSSS (SEQ ID NO: 20) | CDR2: AASSLQS (SEQ ID NO: 5) |
| | CDR2: WYDASN (SEQ ID NO: 23) | CDR3: QQSYSTPLT (SEQ ID |
| | CDR3: RFTYLRGWYIEDYGLDV | NO: 6) |
| | (SEQ ID NO: 24) | |
| AB0900 | EVQLVQSGGGVVQPGRSLRLSC | EIVLTQSPSSLSASVGDRVTITCR |
| | AASGFTFSSSGMHWVRQAPGK | ASQSISSYLNWYQQKPGKAPKLL |
| | CLEWVAVIWYDASNKYYGDSV | IYAASSLQSGVPSRFSGSGSGTDF |
| | KGRFTISRDNSKNTLYLQMNSL | TLTISSLQPEDFATYYCQQSYSTP |
| | RDEDTAVYYCARRFTSLRGWYI | LTFGCGTKVEIK (SEQ ID NO: 63) |
| | EDYGLDVWGQGTTVTVSS (SEQ | CDR1: RASQSISSYLN (SEQ ID |
| | ID NO: 72) | NO: 4) |
| | CDR1: GFTFSSS (SEQ ID NO: 20) | CDR2: AASSLQS (SEQ ID NO: 5) |
| | CDR2: WYDASN (SEQ ID NO: 23) | CDR3: QQSYSTPLT (SEQ ID |
| | CDR3: RFTSLRGWYIEDYGLDV | NO: 6) |
| | (SEQ ID NO: 25) | |
| Consensus 3 | CDR1: GFTFSSS (SEQ ID NO: 20) | CDR1: RASQSISSYLN (SEQ ID |
| (AB0898, AB0899, | CDR2: WYDASN (SEQ ID NO: 23) | NO: 4) |
| AB0900) | CDR3: RFTXLRGWYIEDYGLDV | CDR2: AASSLQS (SEQ ID NO: 5) |
| | where X is R, Y, or S (SEQ ID | CDR3: QQSYSTPLT (SEQ ID |
| | NO: 26) | NO: 6) |
| AB1080scFv | EVQLVQSGGGVVQPGRSLRLSC | EIVLTQSPSSLSASVGDRVTITCR |
| | AASGFTFSSYGMHWVRQAPGK | ASQSISSYLNWYQQKPGKAPKLL |
| | CLEWVAVIWYDASNKYYGDSV | IYAASSLQSGVPSRFSGSGSGTDF |
| | KGRFTISRDNSKNTLYLQMNSL | TLTISSLQPEDFATYYCQQSYSIP |
| | RDEDTAVYYCARRFTHLRGWYI | LTFGCGTKVEIK (SEQ ID NO: 43) |
| | EDYGLDVWGQGTTVTVSS (SEQ | CDR1: RASQSISSYLN (SEQ ID |
| | ID NO: 73) | NO: 4) |
| | CDR1: GFTFSSY (SEQ ID NO: 1) | CDR2: AASSLQS (SEQ ID NO: 5) |
| | CDR2: WYDASN (SEQ ID NO: 23) | CDR3: QQSYSIPLT (SEQ ID |
| | CDR3: RFTHLRGWYIEDYGLDV | NO: 39) |
| | (SEQ ID NO: 27) | |
| AB1081 scFv | EVQLVQSGGGVVQPGRSLRLSC | EIVLTQSPSSLSASVGDRVTITCR |
| | AASGFAFSSYGMHWVRQAPGK | ASQSISSYLNWYQQKPGKAPKLL |
| | CLEWVAVIWYDESNKYYGDSV | IYAASSLQSGVPSRFSGSGSGTDF |
| | KGRFTISRDNSRNTLYLQMNSL | TLTISSLQPEDFATYYCQQSYSIP |
| | RDEDTAVYYCARRFTNLRGWII | LTFGCGTKVEIK (SEQ ID NO: 43) |
| | EDYGLDVWGQGTTVTVSS (SEQ | CDR1: RASQSISSYLN (SEQ ID |
| | ID NO: 74) | NO: 4) |
| | CDR1: GFAFSSY (SEQ ID NO: 28) | CDR2: AASSLQS (SEQ ID NO: 5) |
| | CDR2: WYDESN (SEQ ID NO: 29) | CDR3: QQSYSIPLT (SEQ ID |
| | CDR3: RFTNLRGWIIEDYGLDV | NO: 39) |
| | (SEQ ID NO: 30) | |
| AB1084scFv | EVQLVQSGGGVVQPGRSLRLSC | EIVLTQSPSSLSASVGDRVTITCR |
| | AASGFTFSMYGMHWVRQAPGK | ASQSISSYLNWYQQKPGKAPKLL |
| | CLEWVAVIWYDASNKYYGDSV | IYAASSLQSGVPSRFSGSGSGTDF |
| | KGRFTISRDNSKNTLYLQMNSL | TLTISSLQPEDFATYYCQQSYSIP |
| | RDEDTAVYYCARRFTRLRGWYI | LTFGCGTKVEIK (SEQ ID NO: 43) |
| | EDYGLDVWGQGTTVTVSS (SEQ | CDR1: RASQSISSYLN (SEQ ID |
| | ID NO: 75) | NO: 4) |
| | CDR1: GFTFSMY (SEQ ID NO: 31) | CDR2: AASSLQS (SEQ ID NO: 5) |
| | CDR2: WYDASN (SEQ ID NO: 23) | CDR3: QQSYSIPLT (SEQ ID |
| | CDR3: RFTRLRGWYIEDYGLDV | NO: 39) |
| | (SEQ ID NO: 32) | |
| AB1085scFv | EVQLVQSGGGVVQPGRSLRLSC | EIVLTQSPSSLSASVGDRVTITCR |
| | AASGFTFGSYGMHWVRQAPGK | ASQSISSYLNWYQQKPGKAPKLL |
| | CLEWVAVIWYDGSNKYYGDSV | IYAASSLQSGVPSRFSGSGSGTDF |
| | KGRFTISRDNSKNTLYLQMNSL | TLTISSLQPEDFATYYCQQSYSTP |
| | RDEDTAVYYCARRFTHLRGQYI | LTFGCGTKVEIK (SEQ ID NO: 63) |
| | EDYGMDVWGQGTTVTVSS | CDR1: RASQSISSYLN (SEQ ID |
| | (SEQ ID NO: 76) | NO: 4) |
| | CDR1: GFTFGSY (SEQ ID NO: 33) | CDR2: AASSLQS (SEQ ID NO: 5) |
| | CDR2: WYDGSN (SEQ ID NO: 2) | CDR3: QQSYSTPLT (SEQ ID |
| | CDR3: RFTHLRGQYIEDYGMDV | NO: 6) |
| | (SEQ ID NO: 34) | |
| Consensus 4 | CDR1: GFX₁FX₂X₃Y where X₁ is T | CDR1: RASQSISSYLN (SEQ ID |
| (AB1080, AB1081, | or A, X₂ is S or G, X₃ is S or M | NO: 4) |
| AB1084, AB1085) | (SEQ ID NO: 35) | CDR2: AASSLQS (SEQ ID NO: 5) |
| | CDR2: WYDXSN where X is G, A, | CDR3: QQSYSXPLT where X is T |
| | or E (SEQ ID NO: 36) | or I (SEQ ID NO: 49) |
| | CDR3: | |
| | RFTX₁LRGX₂X₃IEDYGX₄DV | |
| | where X₁ is H, N, or R, X₂ is W or | |
| | Q, X₃ is I or Y, X₄ is M or L (SEQ | |
| | ID NO: 37) | |
| AB1424/AB1612 | EVQLVQSGGGVVQPGRSLRLSC | EIVLTQSPSSLSASVGDRVTITCR |
| | AASGFTFSSYGMHWVRQAPGK | ASQSISSYLNWYQQKPGKAPKL |
| | GLEWVAVIWYDASNKYYGDSV | LIYAASSLQSGVPSRFSGSGSGTD |
| | KGRFTISRDNSKNTLYLQMNSL | FTLTISSLQPEDFATYYCQQSYSI |
| | RDEDTAVYYCARRFTHLRGQY | PLTFGGGTKVEIK (SEQ ID |
| | IEDYGLDVWGQGTTVTVSS | NO: 41) |
| | (SEQ ID NO: 40) | CDR1: RASQSISSYLN (SEQ ID |
| | CDR1: GFTFSSY (SEQ ID NO: 1) | NO: 4) |
| | CDR2: WYDASN (SEQ ID NO: 23) | CDR2: AASSLQS (SEQ ID NO: 5) |
| | CDR3: RFTHLRGQYIEDYGLDV | CDR3: QQSYSIPLT (SEQ ID |
| | (SEQ ID NO: 38) | NO: 39) |
| AB1424/1612 | EVQLVQSGGGVVQPGRSLRLSC | EIVLTQSPSSLSASVGDRVTITCR |
| (with cysteine | AASGFTFSSYGMHWVRQAPGK | ASQSISSYLNWYQQKPGKAPKL |
| heterodimeriza | CLEWVAVIWYDASNKYYGDSV | LIYAASSLQSGVPSRFSGSGSGTD |
| tion mutations | KGRFTISRDNSKNTLYLQMNSL | FTLTISSLQPEDFATYYCQQSYSI |
| for disulfide | RDEDTAVYYCARRFTHLRGQY | PLTFGCGTKVEIK (SEQ ID |
| bridge | IEDYGLDVWGQGTTVTVSS | NO: 43) |
| formation) | (SEQ ID NO: 42) | CDR1: RASQSISSYLN (SEQ ID |
| | CDR1: GFTFSSY (SEQ ID NO: 1) | NO: 4) |
| | CDR2: WYDASN (SEQ ID NO: 23) | CDR2: AASSLQS (SEQ ID NO: 5) |
| | CDR3: RFTHLRGQYIEDYGLDV | CDR3: QQSYSIPLT (SEQ ID |
| | (SEQ ID NO: 38) | NO: 39) |
| AB1424/1612 | EVQLVQSGGGVVQPGRSLRLSCAASGFTFSSYGMHWVRQAPGKCL | |
| scFv (VH-VL) | EWVAVIWYDASNKYYGDSVKGRFTISRDNSKNTLYLQMNSLRDED | |
| | TAVYYCARRFTHLRGQYIEDYGLDVWGQGTTVTVSSGGGGSGGG | |
| | GSGGGGSGGGGSEIVLTQSPSSLSASVGDRVTITCRASQSISSYLNWY | |
| | QQKPGKAPKLLIYAASSLQSGVPSRFSGSGSGTDFTLTISSLQPEDFAT | |
| | YYCQQSYSIPLTFGCGTKVEIK | |
| | (SEQ ID NO: 44) | |
| AB 1424/1612 | EIVLTQSPSSLSASVGDRVTITCRASQSISSYLNWYQQKPGKAPKLLI | |
| scFv (VL-VH) | YAASSLQSGVPSRFSGSGSGTDFTLTISSLQPEDFATYYCQQSYSIPL | |
| | TFGCGTKVEIKGGGGSGGGGSGGGGSGGGGSEVQLVQSGGGVVQP | |
| | GRSLRLSCAASGFTFSSYGMHWVRQAPGKCLEWVAVIWYDASNK | |
| | YYGDSVKGRFTISRDNSKNTLYLQMNSLRDEDTAVYYCARRFTHL | |
| | RGQYIEDYGLDVWGQGTTVTVSS | |
| | (SEQ ID NO: 45) | |
| Consensus 5 | CDR1: GFTFXSY where X is S or | CDR1: RASQSISSYLN (SEQ ID |
| (AB0369, AB1080, | G (SEQ ID NO: 46) | NO: 4) |
| AB1085, AB1424/ | CDR2: WYDXSN where X is G or | CDR2: AASSLQS (SEQ ID NO: 5) |
| AB1612) | A (SEQ ID NO: 47) | CDR3: QQSYSXPLT where X is T |
| | CDR3: | or I (SEQ ID NO: 49) |
| | RFTX₁LRGX₂X₃IEDYGX₄DV | |
| | where X₁ is M or H, X₂ is L, W, or | |
| | Q, X₃ is I or Y, X₄ is M or L (SEQ | |
| | ID NO: 48) | |
| Consensus 6 | CDR1: GX₁X₂FX₃X₄X₅ where X₁ is | CDR1: RASQSISSYLN (SEQ ID |
| (master | For E, X₂ is T, P, W, or A, X₃ is | NO: 4) |
| consensus) | S or G, X₄ is S or M, X₅ is Y or S | CDR2: AASSLQS (SEQ ID NO: 5) |
| | (SEQ ID NO: 50) | CDR3: QQSYSXPLT where X is T |
| | CDR2: WYDXSN where X is G, A, | or I (SEQ ID NO: 49) |
| | or E (SEQ ID NO: 51) | |
| | CDR3: | |
| | RFTX₁LRGX₂X₃IEDYGX₄DV | |
| | where X₁ is M, H, N, R, Y, or S, X₂ | |
| | is L, Q, or W, X₃ is I or Y, X₄ is | |
| | M or L (SEQ ID NO: 52) | |
| 3A1 | QVQLQQPGAELVKPGASVKLSC | DVVMTQTPLSLPVSLGDQASISC |
| | KASGYTFTSYWMHWVKQRPG | RSSQSIVHSNGNTYLEWYLQKP |
| | QGLEWIGEIDPFDSYTNYNQNF | GQSPKLLIYKVSNRLSGVPDRES |
| | KGKATLTVDKSSSTAYMLLSSL | GSGSGTDFTLKISRVEAEDLGVY |
| | TSDDSAVYYCARERLRLWSYY | YCFQGSHDPFTFGSGTKLEIK |
| | FDYWGQGTTLTVSS (SEQ ID | (SEQ ID NO: 55) |
| | NO: 54) | CDR1: RSSQSIVHSNGNTYLE |
| | CDR1: GYTFTSY (SEQ ID NO: 80) | (SEQ ID NO: 83) |
| | CDR2: DPFDSY (SEQ ID NO: 81) | CDR2: KVSNRLS (SEQ ID NO: 84) |
| | CDR3: ERLRLWSYYFDY (SEQ | CDR3: FQGSHDPFT (SEQ ID |
| | ID NO: 82) | NO: 85) |
| 7G4 | QVQLQQPGAELVKPGASVKLSC | DVVMTQTPLSLPVSLGDQASISC |
| | KASGYTFTSYWMHWVKQRPG | RSSQSIVHSNGNTYLEWYLQKP |
| | QGLEWIGEVDPSDSYTNYNQKF | GQSPKLLIYKVSNRLSGVPDRFS |
| | KGKATLTVDKSSSTAYILLSNLT | GSGSGTDFTLKISRVEAEDLGVY |
| | SDDSAVYYCARERVRLWSYFF | YCFQGSHDPFTFGSGTKLEIK |
| | DYWGQGTTLTVSS (SEQ ID | (SEQ ID NO: 57) |
| | NO: 56) | CDR1: RSSQSIVHSNGNTYLE |
| | CDR1: GYTFTSY (SEQ ID NO: 80) | (SEQ ID NO: 83) |
| | CDR2: DPSDSY (SEQ ID NO: 86) | CDR2: KVSNRLS (SEQ ID NO: 84) |
| | CDR3: ERVRLWSYFFDY (SEQ | CDR3: FQGSHDPFT (SEQ ID |
| | ID NO: 87) | NO: 85) |
| 1B3-A7 | EVOLVESGGGLVKPGGSLKLSC | DVVMTQTPLSLPVSPGDQASISC |
| | VVSGFTFSNYAMSWVRQTPEK | RSSQSLVHSNGNTYLYWYLQKP |
| | RLEWVATISDGGGYTYYPDSV | GQSPKLLIYRVSNRFSGVPDRES |
| | KGRFTISRDNAKNNLYLQMSHL | GSGSGTDFTLKINRVEAEDLGVY |
| | KSEDTAIYYCARDDLGGGNYV | FCFQGTHVPLTFGSGTKLELK |
| | SSYFDVWGTGTTVTVSS (SEQ | (SEQ ID NO: 59) |
| | ID NO: 58) | CDR1: RSSQSLVHSNGNTYLY |
| | CDR1: GFTFSNY (SEQ ID NO: 88) | (SEQ ID NO: 91) |
| | CDR2: DGGGY (SEQ ID NO: 89) | CDR2: RVSNRFS (SEQ ID NO: 92) |
| | CDR3: DDLGGGNYVSSYFDV | CDR3: FQGTHVPLT (SEQ ID |
| | (SEQ ID NO: 90) | NO: 93) |
| 10H7-C5 | EVQLVESGGGLVKPGGSLKLSC | DIVMTQTPLSLPVSLGDQASISCR |
| | AASGFSFSRYAMSWVRQTPEK | SSQSLLHSNGNTYLYWYLQKPG |
| | RLEWVATISDGGSYTHYRDNV | QSPKLLIHRVSNRFSGVPDRFGG |
| | KGRFTISRDNAKNNLNLQMSHL | SGSGTDFTLKIIRVEAEDLGVYFC |
| | KSEDTAIYYCARNEMGLYFDY | FQGTHVPWTFGGGTKLEIK (SEQ |
| | DVYAMDYWGQGTSVTVSS | ID NO: 79 |
| | (SEQ ID NO: 60) | CDR1: RSSQSLLHSNGNTYLY |
| | CDR1: GFSFSRY (SEQ ID NO: 94) | (SEQ ID NO: 97) |
| | CDR2: DGGSY (SEQ ID NO: 95) | CDR2: RVSNRFS (SEQ ID NO: 92) |
| | CDR3: NEMGLYFDYDVYAMDY | CDR3: FQGTHVPWT (SEQ ID |
| | (SEQ ID NO: 96) | NO: 53) |

In certain embodiments, the antigen-binding site of the present invention comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 1, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VH of the same antibody disclosed in Table 1. In certain embodiments, the antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), or any other CDR determination method known in the art, of the VH and VL sequences of an antibody discloses in Table 1. In certain embodiments, the antigen-binding site comprises the heavy chain CDR1, CDR2, and CDR3 and the light chain CDR1, CDR2, and CDR3 of an antibody disclosed in Table 1.

In certain embodiments, the antigen-binding site of the present invention comprises a VH comprising CDR1, CDR2, and CDR3 amino acid sequences of SEQ ID NO:50, SEQ ID NO:51, and SEQ ID NO:52, respectively. In certain embodiments, the antigen-binding site of the present invention comprises a VL comprising CDR1, CDR2, and CDR3 amino acid sequences of SEQ ID NO:4, SEQ ID NO:5, and SEQ ID NO:49, respectively. In certain embodiments, the antigen-binding site comprises (a) a VH that comprises CDR1, CDR2, and CDR3 comprising the amino acid sequences of SEQ ID NOs: 50, 51, and 52, respectively; and (b) a VL that comprises CDR1, CDR2, and CDR3 comprising the amino acid sequences of SEQ ID NOs: 4, 5, and 49, respectively.

In certain embodiments, the antigen-binding site of the present invention is derived from AB0369. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:77, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:63. In certain embodiments, the VH comprises CDR1, CDR2, and CDR3 comprising the amino acid sequences of SEQ ID NOs: 1, 2, and 3, respectively. In certain embodiments, the VL comprises CDR1, CDR2, and CDR3 comprising the amino acid sequences of SEQ ID NOs: 4, 5, and 6, respectively. In certain embodiments, the antigen-binding site comprises (a) a VH that comprises CDR1, CDR2, and CDR3 comprising the amino acid sequences of SEQ ID NOs: 1, 2, and 3, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention is derived from 1203_A01. In certain embodiments, the VH comprises CDR1, CDR2, and CDR3 comprising the amino acid sequences of SEQ ID NOs: 1, 2, and 7, respectively. In certain embodiments, the VL comprises CDR1, CDR2, and CDR3 comprising the amino acid sequences of SEQ ID NOs: 8, 9, and 6, respectively. In certain embodiments, the antigen-binding site comprises (a) a VH that comprises CDR1, CDR2, and CDR3 comprising the amino acid sequences of SEQ ID NOs: 1, 2, and 7, respectively; and (b) a VL that comprises CDR1, CDR2, and CDR3 comprising the amino acid sequences of SEQ ID NOs: 8, 9, and 6, respectively.

In certain embodiments, the antigen-binding site of the present invention is derived from 1203_A02. In certain embodiments, the VH comprises CDR1, CDR2, and CDR3 comprising the amino acid sequences of SEQ ID NOs: 10, 2, and 11, respectively. In certain embodiments, the VL comprises CDR1, CDR2, and CDR3 comprising the amino acid sequences of SEQ ID NOs: 4, 5, and 12, respectively. In certain embodiments, the antigen-binding site comprises (a) a VH that comprises CDR1, CDR2, and CDR3 comprising the amino acid sequences of SEQ ID NOs: 10, 2, and 11, respectively; and (b) a VL that comprises CDR1, CDR2, and CDR3 comprising the amino acid sequences of SEQ ID NOs: 4, 5, and 12, respectively.

In certain embodiments, the antigen-binding site of the present invention comprises a VH comprising CDR1, CDR2, and CDR3 amino acid sequences of SEQ ID NO:1, SEQ ID NO:2, and SEQ ID NO:16. In certain embodiments, the antigen-binding site of the present invention comprises a VL comprising CDR1, CDR2, and CDR3 amino acid sequences of SEQ ID NO:4, SEQ ID NO:5, and SEQ ID NO:6, respectively. In certain embodiments, the antigen-binding site comprises (a) a VH that comprises CDR1, CDR2, and CDR3 comprising the amino acid sequences of SEQ ID NOs: 1, 2, and 16, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention is derived from AB0605scFv. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:64, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:63. In certain embodiments, the VH comprises CDR1, CDR2, and CDR3 comprising the amino acid sequences of SEQ ID NOs: 1, 2, and 13, respectively. In certain embodiments, the VL comprises CDR1, CDR2, and CDR3 comprising the amino acid sequences of SEQ ID NOs: 4, 5, and 6, respectively. In certain embodiments, the antigen-binding site comprises (a) a VH that comprises CDR1, CDR2, and CDR3 comprising the amino acid sequences of SEQ ID NOs: 1, 2, and 13, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention is derived from AB0606scFv. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:65, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:63. In certain embodiments, the VH comprises CDR1, CDR2, and CDR3 comprising the amino acid sequences of SEQ ID NOs: 1, 2, and 14, respectively. In certain embodiments, the VL comprises CDR1, CDR2, and CDR3 comprising the amino acid sequences of SEQ ID NOs: 4, 5, and 6, respectively. In certain embodiments, the antigen-binding site comprises (a) a VH that comprises CDR1, CDR2, and CDR3 comprising the amino acid sequences of SEQ ID NOs: 1, 2, and 14, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention is derived from AB0622scFv. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:66, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:63. In certain embodiments, the VH comprises CDR1, CDR2, and CDR3 comprising the amino acid sequences of SEQ ID NOs: 1, 2, and 15, respectively. In certain embodiments, the VL comprises CDR1, CDR2, and CDR3 comprising the amino acid sequences of SEQ ID NOs: 4, 5, and 6, respectively. In certain embodiments, the antigen-binding site comprises (a) a VH that comprises CDR1, CDR2, and CDR3 comprising the amino acid sequences of SEQ ID NOs: 1, 2, and 15, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention comprises a VH comprising CDR1, CDR2, and CDR3 amino acid sequences of SEQ ID NO:21, SEQ ID NO:2, and SEQ ID NO:22. In certain embodiments, the antigen-binding site of the present invention comprises a VL comprising CDR1, CDR2, and CDR3 amino acid sequences of SEQ ID NO:4, SEQ ID NO:5, and SEQ ID NO:6, respectively. In certain embodiments, the antigen-binding site comprises (a) a VH that comprises CDR1, CDR2, and CDR3 comprising the amino acid sequences of SEQ ID NOs: 21, 2, and 22, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention is derived from AB0679scFv. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:67, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:63. In certain embodiments, the VH comprises CDR1, CDR2, and CDR3 comprising the amino acid sequences of SEQ ID NOs: 17, 2 and 14, respectively. In certain embodiments, the VL comprises CDR1, CDR2, and CDR3 comprising the amino acid sequences of SEQ ID NOs: 4, 5, and 6, respectively. In certain embodiments, the antigen-binding site comprises (a) a VH that comprises CDR1, CDR2, and CDR3 comprising the amino acid sequences of SEQ ID NOs: 17, 2, and 14, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention is derived from AB0681scFv. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:68, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:63. In certain embodiments, the VH comprises CDR1, CDR2, and CDR3 comprising the amino acid sequences of SEQ ID NOs: 18, 2, and 19, respectively. In certain embodiments, the VL comprises CDR1, CDR2, and CDR3 comprising the amino acid sequences of SEQ ID NOs: 4, 5, and 6, respectively. In certain embodiments, the antigen-binding site comprises (a) a VH that comprises CDR1, CDR2, and CDR3 comprising the amino acid sequences of SEQ ID NOs: 18, 2, and 19, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention is derived from AB0682scFv. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:69, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:63. In certain embodiments, the VH comprises CDR1, CDR2, and CDR3 comprising the amino acid sequences of SEQ ID NOs: 20, 2, and 14, respectively. In certain embodiments, the VL comprises CDR1, CDR2, and CDR3 comprising the amino acid sequences of SEQ ID NOs: 4, 5, and 6, respectively. In certain embodiments, the antigen-binding site comprises (a) a VH that comprises CDR1, CDR2, and CDR3 comprising the amino acid sequences of SEQ ID NOs: 20, 2, and 14, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention comprises a VH comprising CDR1, CDR2, and CDR3 amino acid sequences of SEQ ID NO:20, SEQ ID NO:23, and SEQ ID NO:26. In certain embodiments, the antigen-binding site of the present invention comprises a VL comprising CDR1, CDR2, and CDR3 amino acid sequences of SEQ ID NO:4, SEQ ID NO:5, and SEQ ID NO:6, respectively. In certain embodiments, the antigen-binding site comprises (a) a VH that comprises CDR1, CDR2, and CDR3 comprising the amino acid sequences of SEQ ID NOs: 20, 23, and 26, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention is derived from AB0898. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:70, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:63. In certain embodiments, the VH comprises CDR1, CDR2, and CDR3 comprising the amino acid sequences of SEQ ID NOs: 20, 23, and 32, respectively. In certain embodiments, the VL comprises CDR1, CDR2, and CDR3 comprising the amino acid sequences of SEQ ID NOs: 4, 5, and 6, respectively. In certain embodiments, the antigen-binding site comprises (a) a VH that comprises CDR1, CDR2, and CDR3 comprising the amino acid sequences of SEQ ID NOs: 20, 23, and 32, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention is derived from AB0899. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:71, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:63. In certain embodiments, the VH comprises CDR1, CDR2, and CDR3 comprising the amino acid sequences of SEQ ID NOs: 20, 23, and 24, respectively. In certain embodiments, the VL comprises CDR1, CDR2, and CDR3 comprising the amino acid sequences of SEQ ID NOs: 20, 23, and 24, respectively. In certain embodiments, the antigen-binding site comprises (a) a VH that comprises CDR1, CDR2, and CDR3 comprising the amino acid sequences of SEQ ID NOs: 20, 23, and 24, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention is derived from AB0900. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:72, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:63. In certain embodiments, the VH comprises CDR1, CDR2, and CDR3 comprising the amino acid sequences of SEQ ID NOs: 20, 23, and 25, respectively. In certain embodiments, the VL comprises CDR1, CDR2, and CDR3 comprising the amino acid sequences of SEQ ID NOs: 4, 5, and 6, respectively. In certain embodiments, the antigen-binding site comprises (a) a VH that comprises CDR1, CDR2, and CDR3 comprising the amino acid sequences of SEQ ID NOs: 20, 23, and 25, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention comprises a VH comprising CDR1, CDR2, and CDR3 amino acid sequences of SEQ ID NO:35, SEQ ID NO:36, and SEQ ID NO:37. In certain embodiments, the antigen-binding site of the present invention comprises a VL comprising CDR1, CDR2, and CDR3 amino acid sequences of SEQ ID NO:4, SEQ ID NO:5, and SEQ ID NO:49, respectively. In certain embodiments, the antigen-binding site comprises (a) a VH that comprises CDR1, CDR2, and CDR3 comprising the amino acid sequences of SEQ ID NOs: 35, 36, and 37, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention is derived from AB1080scFv. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:73, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:43. In certain embodiments, the VH comprises CDR1, CDR2, and CDR3 comprising the amino acid sequences of SEQ ID NOs: 1, 23, and 27, respectively. In certain embodiments, the VL comprises CDR1, CDR2, and CDR3 comprising the amino acid sequences of SEQ ID NOs: 4, 5, and 39, respectively. In certain embodiments, the antigen-binding site comprises (a) a VH that comprises CDR1, CDR2, and CDR3 comprising the amino acid sequences of SEQ ID NOs: 1, 23, and 27, respectively; and (b) a VL that comprises CDR1, CDR2, and CDR3 comprising the amino acid sequences of SEQ ID NOs: 4, 5, and 39, respectively.

In certain embodiments, the antigen-binding site of the present invention is derived from AB1081scFv. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:74, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:43. In certain embodiments, the VH comprises CDR1, CDR2, and CDR3 comprising the amino acid sequences of SEQ ID NOs: 28, 29, and 30, respectively. In certain embodiments, the VL comprises CDR1, CDR2, and CDR3 comprising the amino acid sequences of SEQ ID NOs: 4, 5, and 39, respectively. In certain embodiments, the antigen-binding site comprises (a) a VH that comprises CDR1, CDR2, and CDR3 comprising the amino acid sequences of SEQ ID NOs: 28, 29, and 30, respectively; and (b) a VL that comprises CDR1, CDR2, and CDR3 comprising the amino acid sequences of SEQ ID NOs: 4, 5, and 39, respectively.

In certain embodiments, the antigen-binding site of the present invention is derived from AB 1084scFv. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:75, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:43. In certain embodiments, the VH comprises CDR1, CDR2, and CDR3 comprising the amino acid sequences of SEQ ID NOs: 31, 23, and 32, respectively. In certain embodiments, the VL comprises CDR1, CDR2, and CDR3 comprising the amino acid sequences of SEQ ID NOs: 4, 5, and 39, respectively. In certain embodiments, the antigen-binding site comprises (a) a VH that comprises CDR1, CDR2, and CDR3 comprising the amino acid sequences of SEQ ID NOs: 31, 23, and 32, respectively; and (b) a VL that comprises CDR1, CDR2, and CDR3 comprising the amino acid sequences of SEQ ID NOs: 4, 5, and 39, respectively.

In certain embodiments, the antigen-binding site of the present invention is derived from AB 1085scFv. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 76, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:63. In certain embodiments, the VH comprises CDR1, CDR2, and CDR3 comprising the amino acid sequences of SEQ ID NOs: 33, 2, and 34, respectively. In certain embodiments, the VL comprises CDR1, CDR2, and CDR3 comprising the amino acid sequences of SEQ ID NOs: 4, 5, and 6, respectively. In certain embodiments, the antigen-binding site comprises (a) a VH that comprises CDR1, CDR2, and CDR3 comprising the amino acid sequences of SEQ ID NOs: 33, 2, and 34, respectively; and (b) a VL that comprises CDR1, CDR2, and CDR3 comprising the amino acid sequences of SEQ ID NOs: 4, 5, and 6, respectively.

In certain embodiments, the antigen-binding site of the present invention comprises a VH comprising CDR1, CDR2, and CDR3 amino acid sequences of SEQ ID NO:46, SEQ ID NO:47, and SEQ ID NO:48. In certain embodiments, the antigen-binding site of the present invention comprises a VL comprising CDR1, CDR2, and CDR3 amino acid sequences of SEQ ID NO:4, SEQ ID NO:5, and SEQ ID NO:49, respectively. In certain embodiments, the antigen-binding site comprises (a) a VH that comprises CDR1, CDR2, and CDR3 comprising the amino acid sequences of SEQ ID NOs: 46, 47, and 48, respectively; and (b) a VL that comprises CDR1, CDR2, and CDR3 comprising the amino acid sequences of SEQ ID NOs: 4, 5, and 49, respectively.

In certain embodiments, the antigen-binding site of the present invention is derived from AB1424 or AB1612. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:40, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:41. In certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:42, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:43. In certain embodiments, the VH comprises CDR1, CDR2, and CDR3 comprising the amino acid sequences of SEQ ID NOs: 1, 23, and 38, respectively. In certain embodiments, the VL comprises CDR1, CDR2, and CDR3 comprising the amino acid sequences of SEQ ID NOs: 4, 5, and 39, respectively. In certain embodiments, the antigen-binding site comprises (a) a VH that comprises CDR1, CDR2, and CDR3 comprising the amino acid sequences of SEQ ID NOs: 1, 23, and 38, respectively; and (b) a VL that comprises CDR1, CDR2, and CDR3 comprising the amino acid sequences of SEQ ID NOs: 4, 5, and 39, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 44 or 45.

In certain embodiments, the antigen-binding site of the present invention is derived from 3A1. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:54, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:55. In certain embodiments, the VH comprises CDR1, CDR2, and CDR3 comprising the amino acid sequences of SEQ ID NOs: 80, 81, and 82, respectively. In certain embodiments, the VL comprises CDR1, CDR2, and CDR3 comprising the amino acid sequences of SEQ ID NOs: 83, 84, and 85, respectively. In certain embodiments, the antigen-binding site comprises (a) a VH that comprises CDR1, CDR2, and CDR3 comprising the amino acid sequences of SEQ ID NOS: 80, 81, and 82, respectively; and (b) a VL that comprises CDR1, CDR2, and CDR3 comprising the amino acid sequences of SEQ ID NOs: 83, 84, and 85, respectively.

In certain embodiments, the antigen-binding site of the present invention is derived from 7G4. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:56, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:57. In certain embodiments, the VH comprises CDR1, CDR2, and CDR3 comprising the amino acid sequences of SEQ ID NOs: 80, 86, and 87, respectively. In certain embodiments, the VL comprises CDR1, CDR2, and CDR3 comprising the amino acid sequences of SEQ ID NOS: 83, 84, and 85, respectively. In certain embodiments, the antigen-binding site comprises (a) a VH that comprises CDR1, CDR2, and CDR3 comprising the amino acid sequences of SEQ ID NOs: 80, 86, and 87, respectively; and (b) a VL that comprises CDR1, CDR2, and CDR3 comprising the amino acid sequences of SEQ ID NOs: 83, 84, and 85, respectively.

In certain embodiments, the antigen-binding site of the present invention is derived from 1B3-A7. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:58, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:59. In certain embodiments, the VH comprises CDR1, CDR2, and CDR3 comprising the amino acid sequences of SEQ ID NOs: 88, 89, and 90, respectively. In certain embodiments, the VL comprises CDR1, CDR2, and CDR3 comprising the amino acid sequences of SEQ ID NOs: 91, 92, and 93, respectively. In certain embodiments, the antigen-binding site comprises (a) a VH that comprises CDR1, CDR2, and CDR3 comprising the amino acid sequences of SEQ ID NOs: 88, 89, and 90, respectively; and (b) a VL that comprises CDR1, CDR2, and CDR3 comprising the amino acid sequences of SEQ ID NOs: 91, 92, and 93, respectively.

In certain embodiments, the antigen-binding site of the present invention is derived from 10H7-C5. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:60, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:79. In certain embodiments, the VH comprises CDR1, CDR2, and CDR3 comprising the amino acid sequences of SEQ ID NOs: 94, 95, and 96, respectively. In certain embodiments, the VL comprises CDR1, CDR2, and CDR3 comprising the amino acid sequences of SEQ ID NOs: 97, 92, and 53, respectively. In certain embodiments, the antigen-binding site comprises (a) a VH that comprises CDR1, CDR2, and CDR3 comprising the amino acid sequences of SEQ ID NOs: 94, 95, and 96, respectively; and (b) a VL that comprises CDR1, CDR2, and CDR3 comprising the amino acid sequences of SEQ ID NOs: 97, 92, and 53, respectively.

In each of the foregoing embodiments, it is contemplated herein that the VH and/or VL sequences that together bind BAFF-R may contain amino acid alterations (e.g., at least 1, 2, 3, 4, 5, or 10 amino acid substitutions, deletions, or additions) in the framework regions of the VH and/or VL without affecting their ability to bind to BAFF-R significantly.

In certain embodiments, the antigen-binding site of the present invention binds human BAFF-R with a K_{D} (i.e., dissociation constant) of 1 nM or lower, 5 nM or lower, 10 nM or lower, 15 nM or lower, or 20 nM or lower, as measured by surface plasmon resonance (SPR) (e.g., using the method described in Example 1 infra) or by bio-layer interferometry (BLI), and/or binds BAFF-R from a body fluid, tissue, and/or cell of a subject. In certain embodiments, an antigen-binding site of the present invention has a K_{d} (i.e., off-rate, also called K_{off}) equal to or lower than 1×10⁻⁵, 1×10⁻⁴, 1×10⁻³, 5×10⁻³, 0.01, 0.02, or 0.05 1/s, as measured by SPR (e.g., using the method described in Example 1 infra) or by BLI.

In certain embodiments, the antigen-binding site of the present invention binds cynomolgus BAFF-R with a K_{D} (i.e., dissociation constant) of 5 nM or lower, 10 nM or lower, 15 nM or lower, 20 nM or lower, or 30 nM or lower, as measured by surface plasmon resonance (SPR) (e.g., using the method described in Example 1 infra) or by bio-layer interferometry (BLI), and/or binds BAFF-R from a body fluid, tissue, and/or cell of a subject. In certain embodiments, an antigen-binding site of the present invention has a K_{d} (i.e., off-rate, also called K_{off}) equal to or lower than 1×10⁻³, 5×10⁻³, 0.01, 0.02, or 0.03 1/s, as measured by SPR (e.g., using the method described in Example 1 infra) or by BLI.

In another aspect, the present invention provides an antigen-binding site that competes for binding to BAFF-R (e.g., human BAFF-R) with an antigen-binding site described above. In certain embodiments, the antigen-binding site of the present invention competes with an antigen-binding site derived from AB1424 disclosed above for binding to BAFF-R. In one embodiment, the antigen-binding site competes with AB1424 for binding to BAFF-R. In certain embodiments, the antigen-binding site of the present invention competes with an antigen-binding site derived from a humanized AB1423 disclosed above for binding to BAFF-R. In one embodiment, the antigen-binding site competes with a humanized AB1424 for binding to BAFF-R. In certain embodiments, the antigen-binding site of the present invention competes with an antigen-binding site derived from AB1612 disclosed above for binding to BAFF-R. In one embodiment, the antigen-binding site competes with AB1612 for binding to BAFF-R. In certain embodiments, the antigen-binding site of the present invention competes with an antigen-binding site derived from a humanized AB1612 disclosed above for binding to BAFF-R. In one embodiment, the antigen-binding site competes with a humanized AB1612 for binding to BAFF-R. In certain embodiments, the antigen-binding site of the present invention competes with an antigen-binding site derived from AB0369, 1203_A01, 1203_A02, AB0605, AB0606, AB0622, AB0679, AB0681, AB0682, AB0898, AB0899, AB0900, AB1080, AB1081, AB1084, AB1085, 3A1, 7G4, 1B3-A7, or 10H7-C5 disclosed above for binding to BAFF-R. In some embodiments, the antigen-binding site competes with AB0369, 1203_A01, 1203_A02, AB0605, AB0606, AB0622, AB0679, AB0681, AB0682, AB0898, AB0899, AB0900, AB1080, AB1081, AB1084, AB1085, 3A1, 7G4, 1B3-A7, or 10H7-C5 for binding to BAFF-R.

Proteins with Antigen-Binding Sites

An antigen-binding site disclosed herein can be present in an antibody or antigen-binding fragment thereof. The antibody can be a monoclonal antibody, a chimeric antibody, a diabody, a Fab fragment, a Fab′ fragment, or F(ab′)₂ fragment, an Fv, a bispecific antibody, a bispecific Fab2, a bispecific (mab)2, a humanized antibody, an artificially-generated human antibody, bispecific T-cell engager, bispecific NK cell engager, a single chain antibody (e.g., single-chain Fv fragment or scFv), triomab, knobs-into-holes (kih) IgG with common light chain, crossmab, ortho-Fab IgG, DVD-Ig, 2 in 1-IgG, IgG-scFv, sdFv2-Fc, bi-nanobody, tandAb, dual-affinity retargeting antibody (DART), DART-Fc, scFv-HSA-scFv (where HSA=human serum albumin), or dock-and-lock (DNL)-Fab3. In certain embodiments, an antibody of the present disclosure is an scFv. In certain embodiments, the scFv is in the VH-VL format.

In some embodiments, the single-chain variable fragment (scFv) described above includes a heavy chain variable domain and a light chain variable domain. In some embodiments, the heavy chain variable domain forms a disulfide bridge with the light chain variable domain to enhance stability of the scFv. For example, a disulfide bridge can be formed between the C44 residue of the heavy chain variable domain and the C100 residue of the light chain variable domain, the amino acid positions numbered under Kabat. In some embodiments, the heavy chain variable domain is linked to the light chain variable domain via a flexible linker. Any suitable linker can be used, for example, the (G₄S)₄ linker ((GlyGlyGlyGlySer)₄ (SEQ ID NO:98). In some embodiments of the scFv, the heavy chain variable domain is positioned at the N-terminus of the light chain variable domain. In some embodiments of the scFv, the heavy chain variable domain is positioned at the C terminus of the light chain variable domain.

It is contemplated that in an scFv, a VH and a VL can be connected by a linker, e.g., (GlyGlyGlyGlySer)₄ i.e. (G₄S)₄ linker (SEQ ID NO:98). A skilled person in the art would appreciate that any of the other disclosed linkers (see, e.g., Table 2) may be used in an scFv having a VH and VL sequence disclosed herein (e.g., in Table 1).

The length of the linker (e.g., flexible linker) can be “short,” e.g., 0, 1, 2, 3, 4, 5, 6, 7, 8, 9, 10, 11 or 12 amino acid residues, or “long,” e.g., at least 13 amino acid residues. In certain embodiments, the linker is 10-50, 10-40, 10-30, 10-25, 10-20, 15-50, 15-40, 15-30, 15-25, 15-20, 20-50, 20-40, 20-30, or 20-25 amino acid residues in length.

In certain embodiments, the linker comprises or consists of a (GS)ₙ (SEQ ID NO:109), (GGS)ₙ (SEQ ID NO:110), (GGGS)ₙ (SEQ ID NO:111), (GGSG)ₙ (SEQ ID NO:112), (GGSGG)ₙ (SEQ ID NO:113), and (GGGGS)ₙ (SEQ ID NO:114) sequence, wherein n is 1, 2, 3, 4, 5, 6, 7, 8, 9, 10, 11, 12, 13, 14, 15, 16, 17, 18, 19, or 20. In certain embodiments, the linker comprises or consists of an amino acid sequence selected from SEQ ID NO:98-108 as listed in

TABLE 2

| SEQ ID | Amino Acid Sequence |
|---|---|
| SEQ ID NO: 99 | GSGSGSGSGSGSGSGSGSGS |
| SEQ ID NO: 100 | GGSGGSGGSGGSGGSGGSGGSGGSGGSGGS |
| SEQ ID NO: 101 | GGGSGGGSGGGSGGGSGGGSGGGSGGGSGGGSGGGSGGGS |
| SEQ ID NO: 102 | GGSGGGSGGGSGGGSGGGSGGGSGGGSGGGSGGGSGGGSG |
| SEQ ID NO: 103 | GGSGGGGSGGGGSGGGGSGGGGSGGGGSGGGGSGGGGSGGG |
| | GSGGGGSGG |
| SEQ ID NO: 104 | GGGGSGGGGSGGGGSGGGGSGGGGSGGGGSGGGGSGGGGSG |
| | GGGSGGGGS |
| SEQ ID NO: 98 | GGGGSGGGGSGGGGSGGGGS |
| SEQ ID NO: 105 | GGGGSGGGGSGGGGS |
| SEQ ID NO: 106 | GGGGSGGGGSGGGGSGGGGSGGGGSGGGGSGGGGSGGGGSG |
| | GGGSGGGGSGGGGSGGGGSGGGGSGGGGSGGGGSGGGGSGG |
| | GGSGGGGSGGGGSGGGGS |
| SEQ ID NO: 107 | GGSGGGGSGGGGSGGGGSGGGGSGGGGSGGGGSGGGGSGGG |
| | GSGGGGSGGGGSGGGGSGGGGSGGGGSGGGGSGGGGSGGGGS |
| | GGGGSGGGGSGGGGSGG |
| SEQ ID NO: 108 | SGSGGGGS |

In certain embodiments, an antigen-binding site disclosed herein is linked to an amino acid sequence at least 90% (e.g., 90%, 91%, 92%, 93%, 94%, 95%, 96%, 97%, 98%, 99%, or 100%) identical to an antibody constant region, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, an antigen-binding site disclosed herein can be linked to a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has effector function and can fix complement. In other embodiments the antibody does not recruit effector cells or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In certain embodiments, the antigen-binding site is linked to an IgG constant region including hinge, CH2 and CH3 domains with or without a CH1 domain. In some embodiments, the amino acid sequence of the constant region is at least 90% (e.g., 90%, 91%, 92%, 93%, 94%, 95%, 96%, 97%, 98%, 99%, or 100%) identical to a human antibody constant region, such as an human IgG1 constant region, a human IgG2 constant region, a human IgG3 constant region, or a human IgG4 constant region. In one embodiment, the antibody Fc domain or a portion thereof sufficient to bind CD16 comprises an amino acid sequence at least 90% (e.g., at least 91%, at least 92%, at least 93%, at least 94%, at least 95%, at least 96%, at least 97%, at least 98%, at least 99%, or 100%) identical to wild-type human IgG1 Fc sequence DKTHTCPPCPAPELLGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPG (SEQ ID NO:61). In some other embodiments, the amino acid sequence of the constant region is at least 90% (e.g., at least 91%, at least 92%, at least 93%, at least 94%, at least 95%, at least 96%, at least 97%, at least 98%, at least 99%, or 100%) identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y4071, Y407V, K409F, K409W, K409D, T411D, T411E, K439D, and K439E.

In certain embodiments, the antigen-binding site is linked to a portion of an antibody Fc domain sufficient to bind CD16. Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327 -Ile 332, Leu 234-Ser 239, and carbohydrate residue N-acetyl-D-glucosamine in the CH2 domain (sec, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

In certain embodiments, mutations that can be incorporated into the CHI of a human IgG1 constant region may be at amino acid V125, F126, P127, T135, T139, A140, F170, P171, and/or V173. In certain embodiments, mutations that can be incorporated into the Ck of a human IgG1 constant region may be at amino acid E123, F116, S176, V163, S174, and/or T164.

In some embodiments, the antibody constant domain comprises a CH2 domain and a CH3 domain of an IgG antibody, for example, a human IgG1 antibody. In some embodiments, mutations are introduced in the antibody constant domain to enable heterodimerization with another antibody constant domain. For example, if the antibody constant domain is derived from the constant domain of a human IgG1, the antibody constant domain can comprise an amino acid sequence at least 90% (e.g., at least 91%, at least 92%, at least 93%, at least 94%, at least 95%, at least 96%, at least 97%, at least 98%, at least 99%, or 100%) identical to amino acids 234-332 of a human IgG1 antibody, and differs at one or more positions selected from the group consisting of Q347, Y349, L351, S354, E356, E357, K360, Q362, S364, T366, L368, K370, N390, K392, T394, D399, S400, D401, F405, Y407, K409, T411, and K439. All the amino acid positions in an Fc domain or hinge region disclosed herein are numbered according to EU numbering.

To facilitate formation of an asymmetric protein, Fc domain heterodimerization is contemplated. Mutations (e.g., amino acid substitutions) in the Fc domain that promote heterodimerization are described, for example, in International Application Publication No. WO2019157366, which is not incorporated herein by reference.

The proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the first immunoglobulin light chain can be cloned into a third expression vector; a fourth nucleic acid sequence encoding the second immunoglobulin light chain can be cloned into a fourth expression vector; the first, second, third and fourth expression vectors can be stably transfected together into host cells to produce the multimeric proteins.

To achieve the highest yield of the proteins, different ratios of the first, second, third and fourth expression vectors can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained for expression of a protein comprising an antigen-binding site disclosed herein. The protein can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

Accordingly, in another aspect, the present invention provides one or more isolated nucleic acids comprising sequences encoding an immunoglobulin heavy chain and/or immunoglobulin light chain variable region of any one of the foregoing antibodies. The invention provides one or more expression vectors that express the immunoglobulin heavy chain and/or immunoglobulin light chain variable region of any one of the foregoing antibodies. Similarly, the invention provides host cells comprising one or more of the foregoing expression vectors and/or isolated nucleic acids.

In certain embodiments, the antibody binds BAFF-R with a K_{D} of 25 nM, 20 nM, 15 nM, 10 nM, 9 nM, 8 nM, 7 nM, 6 nM, 5 nM, 4 nM, 3 nM, 2 nM, 1 nM, 0.1 nM or lower, as measured using standard binding assays, for example, surface plasmon resonance or bio-layer interferometry. In certain embodiments, an antibody as disclosed herein binds BAFF-R with a K_{D} less than 5 nM. In certain embodiments the antibody binds BAFF-R from a body fluid, tissue and/or cell of a subject. In certain embodiments, an antibody as disclosed herein inhibits (e.g., blocks) binding of BAFF-R to BAFF (e.g., by at least 50%, 75%, 90%, 95% or 99% as measured in a competitive binding assay).

Competition assays for determining whether an antibody binds to the same epitope as, or competes for binding with a disclosed antibody are known in the art. Exemplary competition assays include immunoassays (e.g., ELISA assays, RIA assays), surface plasmon resonance (e.g., BIAcore analysis), bio-layer interferometry, and flow cytometry.

Typically, a competition assay involves the use of an antigen (e.g., a human BAFF-R protein or fragment thereof) bound to a solid surface or expressed on a cell surface, a test BAFF-R-binding antibody and a reference antibody. The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (e.g., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated human antibodies contain one or more somatic mutations. In these cases, antibodies can be modified to a human germline sequence to optimize the antibody (e.g., by a process referred to as germlining).

Generally, an optimized antibody has at least the same, or substantially the same, affinity for the antigen as the non-optimized (or parental) antibody from which it was derived. Preferably, an optimized antibody has a higher affinity for the antigen when compared to the parental antibody.

If the antibody is for use as a therapeutic, it can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

The antibody can be conjugated to an effector moiety such as a small molecule toxin or a radionuclide using standard in vitro conjugation chemistries. If the effector moiety is a polypeptide, the antibody can be chemically conjugated to the effector or joined to the effector as a fusion protein. Construction of fusion proteins is within ordinary skill in the art.

### CAR T Cells, BAFF-R/CD3-Directed Bispecific T-Cell Engagers, Immunocytokines, Antibody-Drug Conjugates, and Immunotoxins

Another aspect of the present invention provides a molecule or complex comprising an antigen-binding site that binds BAFF-R as disclosed herein. Exemplary molecules or complexes include but are not limited to chimeric antigen receptors (CARs), T-cell engagers (e.g., BAFF-R/CD3-directed bispecific T-cell engagers), immunocytokines, antibody-drug conjugates, and immunotoxins.

Any antigen-binding site that binds BAFF-R as disclosed herein can be used. In certain embodiments, the VH, VL, and/or CDR sequences of the antigen-binding site that binds BAFF-R are provided in Table 1. In certain embodiments, the antigen-binding site that binds BAFF-R is an scFv. In certain embodiments, the scFv comprises an amino acid sequence at least 90% (e.g., at least 91%, at least 92%, at least 93%, at least 94%, at least 95%, at least 96%, at least 97%, at least 98%, or at least 99%) identical to an amino acid sequence selected from SEQ ID NOs: 44 and 45. In certain embodiments, the scFv comprises an amino acid sequence selected from SEQ ID NOs: 44 and 45.

In certain embodiments, the antigen-binding site that binds BAFF-R in the molecule or complex (e.g., CAR, T-cell engager, immunocytokine, antibody-drug conjugate, or immunotoxin) comprises a heavy chain variable domain comprising CDR1, CDR2, and CDR3 sequences represented by the amino acid sequences of SEQ ID NOs: 1, 23, and 38, respectively; and a light chain variable domain comprising CDR1, CDR2, and CDR3 sequences represented by the amino acid sequences of SEQ ID NOs: 4, 5, and 39, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:40 or 42; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID

NO:41 or 43. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:44 or SEQ ID NO:45. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:44.

### Chimeric Antigen Receptors (CARs)

In certain embodiments, the present invention provides a BAFF-R-targeting CAR comprising an antigen-binding site that binds BAFF-R as disclosed herein (see, e.g., Table 1). The BAFF-R-targeting CAR can comprise a Fab fragment or an scFv.

The term “chimeric antigen receptor” or alternatively a “CAR” refers to a recombinant polypeptide construct comprising at least an extracellular antigen binding domain, a transmembrane domain and an intracellular signaling domain comprising a functional signaling domain derived from a stimulatory molecule (also referred to herein as a “primary signaling domain”).

Accordingly, in certain embodiments, the CAR comprises an extracellular antigen-binding site that binds BAFF-R as disclosed herein, a transmembrane domain, and an intracellular signaling domain comprising a primary signaling domain. In certain embodiments, the CAR further comprises one or more functional signaling domains derived from at least one costimulatory molecule (also referred to as a “costimulatory signaling domain”).

In certain embodiments, the CAR comprises a chimeric fusion protein comprising an antigen-binding site that binds BAFF-R (e.g., BAFF-R-binding scFv) disclosed herein as an extracellular antigen binding domain, a transmembrane domain, and an intracellular signaling domain comprising a primary signaling domain. In certain embodiments, the CAR comprises a chimeric fusion protein comprising an antigen-binding site that binds BAFF-R (e.g., BAFF-R-binding scFv) disclosed herein as an extracellular antigen binding domain, a transmembrane domain, and an intracellular signaling domain comprising a costimulatory signaling domain and a primary signaling domain. In certain embodiments, the CAR comprises a chimeric fusion protein comprising an antigen-binding site that binds BAFF-R (e.g., BAFF-R-binding scFv) disclosed herein as an extracellular antigen binding domain, a transmembrane domain, and an intracellular signaling domain comprising two costimulatory signaling domains and a primary signaling domain. In certain embodiments, the CAR comprises a chimeric fusion protein comprising an antigen-binding site that binds BAFF-R (e.g., BAFF-R-binding scFv) disclosed herein as an extracellular antigen binding domain, a transmembrane domain, and an intracellular signaling domain comprising at least two costimulatory signaling domains and a primary signaling domain.

For example, in certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 1, 23, and 38, respectively; and a light chain variable domain comprising CDR1, CDR2, and CDR3 sequences represented by the amino acid sequences of SEQ ID NOs: 4, 5, and 39, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:40 or 42; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:41 or 43. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:44 or SEQ ID NO:45. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:44.

With respect to the transmembrane domain, in various embodiments, the CAR is designed to comprise a transmembrane domain that is fused to the extracellular domain of the CAR. In one embodiment, the transmembrane domain is one that naturally is associated with one of the domains in the CAR. In some instances, the transmembrane domain can be selected or modified by amino acid substitution to avoid binding of such domains to the transmembrane domains of the same or different surface membrane proteins to minimize interactions with other members of the receptor complex. In another embodiment, the transmembrane domain is capable of homodimerization with another CAR on the CAR T cell surface. In another embodiment, the amino acid sequence of the transmembrane domain may be modified or substituted so as to minimize interactions with the binding domains of the native binding partner present in the same CAR T cell.

The transmembrane domain may be derived from any naturally occurring membrane-bound or transmembrane protein. In one embodiment, the transmembrane region is capable of signaling to the intracellular domain(s) whenever the CAR has bound to a target. In some embodiments, the transmembrane domain comprises the transmembrane region(s) of one or more proteins selected from the group consisting of TCR a chain, TCR B chain, TCR & chain, CD28, CD38, CD45, CD4, CD5, CD8, CD9, CD16, CD22, BAFF-R, CD37, CD64, CD80, CD86, CD134, CD137, and CD154. In some embodiments, the transmembrane domain comprises the transmembrane region(s) of one or more proteins selected from the group consisting of KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NKG2C.

The extracellular BAFF-R-binding domain (e.g., BAFF-R-binding scFv domain) domain can be connected to the transmembrane domain by a hinge region. A variety of hinges can be employed, including but not limited to the human Ig (immunoglobulin) hinge (e.g., an IgG4 hinge, an IgD hinge), a Gly-Ser linker, a (G₄S)₄ linker, a KIR2DS2 hinge, and a CD8α hinge.

The intracellular signaling domain of the CAR of the present invention is responsible for activation of at least one of the specialized functions of the immune cell (e.g., cytolytic activity or helper activity, including the secretion of cytokines, of a T cell) in which the CAR has been placed in. Thus, as used herein, the term “intracellular signaling domain” refers to the portion of a protein which transduces an effector function signal and directs the cell to perform a specialized function. Although usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain of the CAR comprises a primary signaling domain (i.e., a functional signaling domain derived from a stimulatory molecule) and one or more costimulatory signaling domains (i.e., functional signaling domains derived from at least one costimulatory molecule).

As used herein, the term “stimulatory molecule” refers to a molecule expressed by an immune cell, e.g., a T cell, an NK cell, or a B cell, that provide the cytoplasmic signaling sequence(s) that regulate activation of the immune cell in a stimulatory way for at least some aspect of the immune cell signaling pathway. In one embodiment, the signal is a primary signal that is initiated by, for instance, binding of a TCR/CD3 complex with an MHC molecule loaded with a peptide, and which leads to mediation of a T cell response, including, but not limited to, proliferation, activation, differentiation, and the like.

Primary signaling domains that act in a stimulatory manner may contain signaling motifs which are known as immunoreceptor tyrosine-based activation motifs or ITAMs. Examples of ITAM containing cytoplasmic signaling sequences that are of particular use in the present invention include those derived from CD3 zeta, common FcR gamma (FCERIG), Fc gamma RIIa, FcR beta (Fc Epsilon R1b), CD3 gamma, CD3 delta, CD3 epsilon, CD79a, CD79b, DAP10, and DAP12. In one embodiment, the primary signaling domain in any one or more CARs of the present invention comprises a cytoplasmic signaling sequence derived from CD3-zeta.

In some embodiments, the primary signaling domain is a functional signaling domain of TCR zeta, FcR gamma, FcR beta, CD3 gamma, CD3 delta, CD3 epsilon, CD5, CD22, CD79a, CD79b, CD66d, 4-1BB, and/or CD3-zeta. In an embodiment, the intracellular signaling domain comprises a functional signaling domain of CD3 zeta, common FcR gamma (FCERIG), Fc gamma RIIa, FcR beta (Fc Epsilon R1b), CD3 gamma, CD3 delta, CD3 epsilon, CD79a, CD79b, DAP10, and/or DAP12. In a particular embodiment, the primary signaling domain is a functional signaling domain of the zeta chain associated with the T cell receptor complex.

As used herein, the term “costimulatory molecule” refers to a cognate binding partner on a T cell that specifically binds with a costimulatory ligand, thereby mediating a costimulatory response by the T cell, such as, but not limited to, proliferation. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1, CD11a/CD18), CD2, CD7, CD258 (LIGHT), NKG2C, B7-H3, and a ligand that specifically binds with CD83, and the like. Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD1la, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and a ligand that specifically binds with CD83. In some embodiments, the costimulatory signaling domain of the CAR is a functional signaling domain of a costimulatory molecule described herein, e.g., OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

As used herein, the term “signaling domain” refers to the functional portion of a protein which acts by transmitting information within the cell to regulate cellular activity via defined signaling pathways by generating second messengers or functioning as effectors by responding to such messengers.

The cytoplasmic signaling sequences within the cytoplasmic signaling portion of the CAR of the present invention may be linked to each other in a random or specified order. Optionally, a short oligo- or polypeptide linker, for example, between 2 and 10 amino acids in length may form the linkage.

Another aspect of the present invention provides a nucleic acid encoding a BAFF-R-targeting CAR disclosed herein. The nucleic acid is useful for expressing the CAR in an effector cell (e.g., T cell) by introducing the nucleic acid to the cell.

Modifications may be made in the sequence to create an equivalent or improved variant of the present invention, for example, by changing one or more of the codons according to the codon degeneracy table. A DNA codon degeneracy table is provided in Table 3.

TABLE 3
Amino Acid Codons

| | One | Three | |
|---|---|---|---|
| | letter | letter | |
| Amino Acids | code | code | Codons |
| Alanine | A | Ala | GCA GCC GCG GCU |
| Cysteine | C | Cys | UGC UGU |
| Aspartic acid | D | Asp | GAC GAU |
| Glutamic acid | E | Glu | GAA GAG |
| Phenylalanine | F | Phe | UUC UUU |
| Glycine | G | Gly | GGA GGC GGG GGU |
| Histidine | H | His | CAC CAU |
| Isoleucine | I | Iso | AUA AUC AUU |
| Lysine | K | Lys | AAA AAG |
| Leucine | L | Leu | UUA UUG CUA CUC CUG CUU |
| Methionine | M | Met | AUG |
| Asparagine | N | Asn | AAC AAU |
| Proline | P | Pro | CCA CCC CCG CCU |
| Glutamine | Q | Gln | CAA CAG |
| Arginine | R | Arg | AGA AGG CGA CGC CGG CGU |
| Serine | S | Ser | AGC AGU UCA UCC UCG UCU |
| Threonine | T | Thr | ACA ACC ACG ACU |
| Valine | V | Val | GUA GUC GUG GUU |
| Tryptophan | W | Trp | UGG |
| Tyrosine | Y | Tyr | UAC UAU |

In certain embodiments, the nucleic acid is a DNA molecule (e.g., a cDNA molecule). In certain embodiments, the nucleic acid further comprises an expression control sequence (e.g., promoter and/or enhancer) operably linked to the CAR coding sequence. In certain embodiments, the present invention provides a vector comprising the nucleic acid. The vector can be a viral vector (e.g., AAV vector, lentiviral vector, or adenoviral vector) or a non-viral vector (e.g., plasmid).

In certain embodiments, the nucleic acid is an RNA molecule (e.g., an mRNA molecule). A method for generating mRNA for use in transfection can involve in vitro transcription of a template with specially designed primers, followed by polyA addition, to produce an RNA construct containing 3′ and 5′ untranslated sequences, a 5′ cap and/or Internal Ribosome Entry Site (IRES), the nucleic acid to be expressed, and a polyA tail, typically 50-2000 bases in length. The RNA molecule can be further modified to increase translational efficiency and/or stability, e.g., as disclosed in U.S. Pat. Nos. 8,278,036; 8,883,506, and 8,716,465. RNA molecules so produced can efficiently transfect different kinds of cells.

In one embodiment, the nucleic acid encodes an amino acid sequence comprising a signal peptide at the amino-terminus of the CAR. Such signal peptide can facilitate the cell surface localization of the CAR when it is expressed in an effector cell, and is cleaved from the CAR during cellular processing. In one embodiment, the nucleic acid encodes an amino acid sequence comprising a signal peptide at the N-terminus of the extracellular BAFF-R-binding domain (e.g., BAFF-R-binding scFv domain).

RNA or DNA can be introduced into target cells using any of a number of different methods, for instance, commercially available methods which include, but are not limited to, electroporation, cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001)).

Another aspect of the present invention provides an immune effector cell expressing the BAFF-R-targeting CAR. Also provided is an immune effector cell comprising the nucleic acid encoding the BAFF-R-targeting CAR. The immune effector cells include but are not limited to T cells and NK cells. In certain embodiments, the T cell is selected from a CD8⁺ T cell, a CD4⁺ T cell, and an NKT cell. The T cell or NK cell can be a primary cell or a cell line.

The immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors, by methods known in the art. The immune effector cells can also be differentiated in vitro from a pluripotent or multipotent cell (e.g., a hematopoietic stem cell). In some embodiments, the present invention provides a pluripotent or multipotent cell (e.g., a hematopoietic stem cell) expressing the BAFF-R-targeting CAR (e.g., expressing the CAR on the plasma membrane) or comprising a nucleic acid disclosed herein.

In certain embodiments, the immune effector cells are isolated and/or purified. For example, regulatory T cells can be removed from a T cell population using a CD25-binding ligand. Effector cells expressing a checkpoint protein (e.g., PD-1, LAG-3, or TIM-3) can be removed by similar methods. In certain embodiments, the effector cells are isolated by a positive selection step. For example, a population of T cells can be isolated by incubation with anti-CD3/anti-CD28-conjugated beads. Other cell surface markers, such as IFN-7, TNF-α, IL-17A, IL-2, IL-3, IL-4, GM-CSF, IL-10, IL-13, granzyme B, and perforin, can also be used for positive selection.

Immune effector cells may be activated and expanded generally using methods known in the art, e.g., as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publications Nos. 2006/0121005 and 2016/0340406. For example, in certain embodiments, T cells can be expanded and/or activated by contact with an anti-CD3 antibody and an anti-CD28 antibody, under conditions appropriate for stimulating proliferation of the T cells. The cells can be expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. Multiple cycles of stimulation may be desirable for prolonged cell culture (e.g., culture for a period of 60 days or more). In certain embodiments, the cell culture comprises serum (e.g., fetal bovine or human serum), interleukin-2 (IL-2), insulin, IFN-γ, IL-4, IL-7, GM-CSF, IL-10, IL-12, IL-15, TGFβ, TNF-α, or a combination thereof. Other additives for the growth of cells known to the skilled person, e.g., surfactant, plasmanate, and reducing agents such as N-acetyl-cysteine and 2-mercaptoethanol, can also be included in the cell culture. In certain embodiments, the immune effector cell of the present invention is a cell obtained from in vitro expansion.

Further embodiments of the BAFF-R-targeting CAR (e.g., regulatable CAR), nucleic acid encoding the CAR, and effector cells expressing the CAR or comprising the nucleic acid are provided in U.S. Pat. Nos. 7,446,190 and 9,181,527, U.S. Patent Application Publication Nos. 2016/0340406 and 2017/0049819, and International Patent Application Publication No. WO2018/140725.

### BAFF-R/CD3-Directed Bispecific T-Cell Engagers

In certain embodiments, the present invention provides a BAFF-R/CD3-directed bispecific T-cell engager comprising an antigen-binding site that binds BAFF-R disclosed herein. In certain embodiments, the BAFF-R/CD3-directed bispecific T-cell engager comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 44 and 45. In certain embodiments, the cytokine is connected to the Fc domain directly or via a linker.

In certain embodiments, the BAFF-R/CD3-directed bispecific T-cell engager further comprises an antigen-binding site that binds CD3. Exemplary antigen-binding sites that bind CD3 are disclosed in International Patent Application Publication Nos. WO2014/051433 and WO2017/097723.

Another aspect of the present invention provides a nucleic acid encoding at least one polypeptide of the BAFF-R/CD3-directed bispecific T-cell engager, wherein the polypeptide comprises an antigen-binding site that binds BAFF-R. In certain embodiments, the nucleic acid further comprises a nucleotide sequence encoding a signal peptide that, when expressed, is at the N-terminus of one or more of the polypeptides of the BAFF-R/CD3-directed bispecific T-cell engager. Also provided is a vector (e.g., a viral vector) comprising the nucleic acid, a producer cell comprising the nucleic acid or vector, and a producer cell expressing the BAFF-R/CD3-directed bispecific T-cell engager.

### Immunocytokines

In certain embodiments, the present invention provides an immunocytokine comprising an antigen-binding site that binds BAFF-R disclosed herein and a cytokine. Any cytokine (e.g., pro-inflammatory cytokines) known in the art can be used, including but not limited to IL-2, IL-4, IL-10, IL-12, IL-15, TNF, IFNα, IFNγ, and GM-CSF. More exemplary cytokines are disclosed in U.S. Pat. No. 9,567,399. In certain embodiments, the antigen-binding site is connected to the cytokine by chemical conjugation (e.g., covalent or noncovalent chemical conjugation). In certain embodiments, the antigen-binding site is connected to the cytokine by fusion of polypeptide. The immunocytokine can further comprise an Fc domain connected to the antigen-binding site that binds BAFF-R. In certain embodiments, the immunocytokine comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 44 and 45. In certain embodiments, the cytokine is connected to the Fc domain directly or via a linker.

Another aspect of the present invention provides a nucleic acid encoding at least one polypeptide of the immunocytokine, wherein the polypeptide comprises an antigen-binding site that binds BAFF-R. In certain embodiments, the nucleic acid further comprises a nucleotide sequence encoding a signal peptide that, when expressed, is at the N-terminus of one or more of the polypeptides of the immunocytokine. Also provided is a vector (e.g., a viral vector) comprising the nucleic acid, a producer cell comprising the nucleic acid or vector, and a producer cell expressing the immunocytokine.

### Antibody-Drug Conjugates

In certain embodiments, the present invention provides an antibody-drug conjugate comprising an antigen-binding site that binds BAFF-R disclosed herein and a cytotoxic drug moiety. Exemplary cytotoxic drug moieties are disclosed in International Patent Application Publication Nos. WO2014/160160 and WO2015/143382. In certain embodiments, the cytotoxic drug moiety is selected from auristatin, N-acetyl-γ calicheamicin, maytansinoid, pyrrolobenzodiazepine, and SN-38. The antigen-binding site can be connected to the cytotoxic drug moiety by chemical conjugation (e.g., covalent or noncovalent chemical conjugation). In certain embodiments, the antibody-drug conjugate further comprises an Fc domain connected to the antigen-binding site that binds BAFF-R. In certain embodiments, the antibody-drug conjugate comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 44 and 45. In certain embodiments, the cytotoxic drug moiety is connected to the Fc domain directly or via a linker.

### Immunotoxins

In certain embodiments, the present invention provides an immunotoxin comprising an antigen-binding site that binds BAFF-R disclosed herein and a cytotoxic peptide moiety. Any cytotoxic peptide moiety known in the art can be used, including but not limited to ricin, Diphtheria toxin, and Pseudomonas exotoxin A. More exemplary cytotoxic peptides are disclosed in International Patent Application Publication Nos. WO2012/154530 and WO2014/164680. In certain embodiments, the cytotoxic peptide moiety is connected to the protein by chemical conjugation (e.g., covalent or noncovalent chemical conjugation). In certain embodiments, the cytotoxic peptide moiety is connected to the protein by fusion of polypeptide. The immunotoxin can further comprise an Fc domain connected to the antigen-binding site that binds BAFF-R. In certain embodiments, the immunotoxin comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 44 and 45. In certain embodiments, the cytotoxic peptide moiety is connected to the Fc domain directly or via a linker.

Another aspect of the present invention provides a nucleic acid encoding at least one polypeptide of the immunotoxin, wherein the polypeptide comprises an antigen-binding site that binds BAFF-R. In certain embodiments, the nucleic acid further comprises a nucleotide sequence encoding a signal peptide that, when expressed, is at the N-terminus of one or more of the polypeptides of the immunotoxin. Also provided is a vector (e.g., a viral vector) comprising the nucleic acid, a producer cell comprising the nucleic acid or vector, and a producer cell expressing the immunotoxin.

### II. Therapeutic Compositions and Their Use

The present invention provides methods for treating cancer or autoimmune disease using a protein, conjugate, or cells comprising an antigen-binding site disclosed herein and/or a pharmaceutical composition described herein. The methods may be used to treat a variety of cancers which express BAFF-R by administering to a patient in need thereof a therapeutically effective amount of a protein, conjugate, or cells comprising an antigen-binding site disclosed herein.

The therapeutic method can be characterized according to the cancer to be treated. The cancer to be treated can be characterized according to the presence of a particular antigen expressed on the surface of the cancer cell, e.g., BAFF-R.

Cancers characterized by the expression of BAFF-R, include, without limitation, B-cell non-Hodgkin's lymphoma (B-NHL), such as chronic lymphocytic leukemia (CLL), mantle cell lymphoma (MCL), follicular lymphoma (FL), diffuse large B-cell lymphoma (DLBCL), marginal zone lymphoma, mucosa-associated lymphoid tissue (MALT) lymphoma, primary mediastinal B-cell lymphoma, acute lymphocytic leukemia (ALL); and autoimmune inflammatory diseases.

It is contemplated that the protein, conjugate, cells, and/or pharmaceutical compositions described in the present disclosure can be used to treat a variety of cancers, not limited to cancers in which the cancer cells or the cells in the cancer microenvironment express BAFF-R.

In certain embodiments, the cancer is a solid tumor. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, Bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, intraepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In certain embodiments, the cancer is a hematologic malignancy. In certain embodiments, the hematologic malignancy is leukemia. In certain embodiments, selected from the group consisting of acute myeloid leukemia (AML), acute lymphoblastic leukemia (ALL), myelodysplasia, myelodysplastic syndromes, acute T-lymphoblastic leukemia, or acute promyelocytic leukemia, chronic myelomonocytic leukemia, or myeloid blast crisis of chronic myeloid leukemia.

In some embodiments, the present application provides methods for treating an autoimmune inflammatory disease using a protein described herein and/or a pharmaceutical composition described herein. The methods may be used to treat a variety of BAFF-R-expressing B cell-associated autoimmune inflammatory diseases, including, without limitation, multiple sclerosis, systemic lupus erythematosus, Graves' disease, Hashimoto's thyroiditis, rheumatoid arthritis, inflammatory bowel disease, type I diabetes, Guillain-Barre syndrome, chronic inflammatory demyelinating polyneuropathy, psoriasis, myasthenia gravis, and vasculitis.

### III. Pharmaceutical Compositions

Another aspect of the present application provides for combination therapy. A protein described herein can be used in combination with additional therapeutic agents to treat autoimmune disease or to treat cancer.

Exemplary therapeutic agents that may be used as part of a combination therapy in treating autoimmune inflammatory diseases are described in Li et al. (2017) Front. Pharmacol., 8:460, and include, for example, non-steroidal anti-inflammatory drugs (NSAIDs) (e.g., COX-2 inhibitors), glucocorticoids (e.g., prednisone/prednisolone, methylprednisolone, and the fluorinated glucocorticoids such as dexamethasone and betamethasone), disease-modifying antirheumatic drugs (DMARDs) (e.g., methotrexate, leflunomide, gold compounds, sulfasalazine, azathioprine, cyclophosphamide, antimalarials, D-penicillamine, and cyclosporine), anti-TNF biologics (e.g., infliximab, etanercept, adalimumab, golimumab, Certolizumab pegol, and their biosimilars), and other biologics targeting CTLA-4 (e.g., abatacept), IL-6 receptor (e.g., tocilizumab), IL-1 (e.g., anakinra), Th1 immune responses (IL-12/IL-23) (e.g., ustekinumab), Th17 immune responses (IL-17) (e.g., secukinumab) and CD20 (e.g., rituximab).

Exemplary therapeutic agents that may be used as part of a combination therapy in treating cancer include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma (IFN-γ), colony stimulating factor-1, colony stimulating factor-2, denileukin diftitox, interleukin-2, luteinizing hormone releasing factor and variations of the aforementioned agents that may exhibit differential binding to its cognate receptor, or increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Ab1 Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTHI Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the present disclosure can also be used as an adjunct to surgical removal of the primary lesion.

The amount of protein and additional therapeutic agent, and the relative timing of administration, may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a protein may be administered during a time when the additional therapeutic agent(s) exerts its prophylactic or therapeutic effect, or vice versa.

### IV. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, sec, e.g., Langer (Science 249:1527-1533, 1990).

In one aspect, the present disclosure provides a formulation of a protein, which contains a BAFF-R-binding site described herein, and a pharmaceutically acceptable carrier.

In certain embodiments, the pharmaceutical composition includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:40, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:41. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:42, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:43. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:54, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:55. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:56, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:57. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:59, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:59. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:60, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:79. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:77, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:64, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:65, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:66, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:67, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:68, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:69, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:70, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:71, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:72, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:73, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:74, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:75, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:76, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:63.

The composition can be formulated for use in a variety of drug delivery systems. One or more physiologically acceptable excipients or carriers can be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

For example, this present disclosure could exist in an aqueous pharmaceutical formulation including a therapeutically effective amount of the protein in a buffered solution forming a formulation. Aqueous carriers can include sterile water for injection (SWFI), bacteriostatic water for injection (BWFI), a pH buffered solution (e.g. phosphate-buffered saline), sterile saline solution, Ringer's solution or dextrose solution. In certain embodiments, an aqueous formulation is prepared including the protein disclosed herein in a pH-buffered solution. The pH of the preparations typically will be between 3 and 11, more preferably between 5 and 9 or between 6 and 8, and most preferably between 7 and 8, such as 7 to 7.5. Ranges intermediate to the above recited pH's are also intended to be part of this disclosure. For example, ranges of values using a combination of any of the above recited values as upper and/or lower limits are intended to be included. Examples of buffers that will control the pH within this range include acctate (e.g., sodium acetate), succinate (such as sodium succinate), gluconate, histidine, citrate and other organic acid buffers. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g. 1.53 mg/mL), about 0.9 mg/mL of sodium dihydrogen phosphate dihydrate (e.g., 0.86), and about 6.2 mg/mL of sodium chloride (e.g., 6.165 mg/mL). In certain embodiments, the buffer system includes 1-1.5 mg/mL of citric acid, 0.25 to 0.5 mg/mL of sodium citrate, 1.25 to 1.75 mg/ml of disodium phosphate dihydrate, 0.7 to 1.1 mg/mL of sodium dihydrogen phosphate dihydrate, and 6.0 to 6.4 mg/mL of sodium chloride. The pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In some embodiments, the formulation include an aqueous carrier, which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to about 15 mg/mL. In certain embodiments, the concentration of mannitol may be about 10 to about 14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative, which is added to the formulations herein to reduce bacterial action. The addition of a preservative may, for example, facilitate the production of a multi-use (multiple-dose) formulation.

In some embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

Deamidation is a common product variant of peptides and proteins that may occur during fermentation, harvest/cell clarification, purification, drug substance/drug product storage and during sample analysis. Deamidation is the loss of NH3 from a protein forming a succinimide intermediate that can undergo hydrolysis. The succinimide intermediate results in a 17 dalton mass decrease of the parent peptide. The subsequent hydrolysis results in an 18 dalton mass increase. Isolation of the succinimide intermediate is difficult due to instability under aqueous conditions. As such, deamidation is typically detectable as 1 dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation. In certain embodiments, the liquid formulation of the present disclosure may be preserved under conditions of pH and humidity to prevent deamination of the protein product.

In some embodiment, the formulation is a lyophilized formulation. In certain embodiments, the formulation is freeze-dried (lyophilized) and contained in about 12-60 vials. In certain embodiments, the formulation is freeze-dried and 45 mg of the freeze-dried formulation may be contained in one vial. In certain embodiments, the about 40 mg—about 100 mg of freeze-dried formulation is contained in one vial. In certain embodiments, freeze dried formulation from 12, 27, or 45 vials are combined to obtained a therapeutic dose of the protein in the intravenous drug formulation. The formulation may be a liquid formulation. In some embodiments, a liquid formulation is stored as about 250 mg/vial to about 1000 mg/vial. In certain embodiments, the liquid formulation is stored as about 600 mg/vial. In certain embodiments, the liquid formulation is stored as about 250 mg/vial.

In some embodiments, the lyophilized formulation includes the proteins described herein and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative. The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide. Before lyophilization, the pH of the solution containing the protein of the present disclosure may be adjusted between 6 to 8. In certain embodiments, the pH range for the lyophilized drug product may be from 7 to 8.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present disclosure may contain such bulking agents.

In certain embodiments, the lyophilized protein product is constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution. In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution. In certain embodiments, the lyophilized protein product of the instant disclosure is constituted to about 4.5 mL water for injection and diluted with 0.9% saline solution (sodium chloride solution).

The protein compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The resulting compositions in solid form may be packaged in multiple single dose units, cach containing a fixed amount of the above-mentioned agent or agents. The composition in solid form can also be packaged in a container for a flexible quantity.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica. Chimica. Acta. 308: 43-53, 2001; Steimer et al., Clinica. Chimica. Acta. 308: 33-41, 2001).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight. Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present disclosure could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of the present disclosure. The patent application specifically contemplates all combinations and permutations of the aspects and embodiments.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure(s). For example, it will be appreciated that all features described and depicted herein can be applicable to all aspects of the disclosure(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present disclosure remain operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1. Generation and Characterization of BAFF-R Binding mAbs

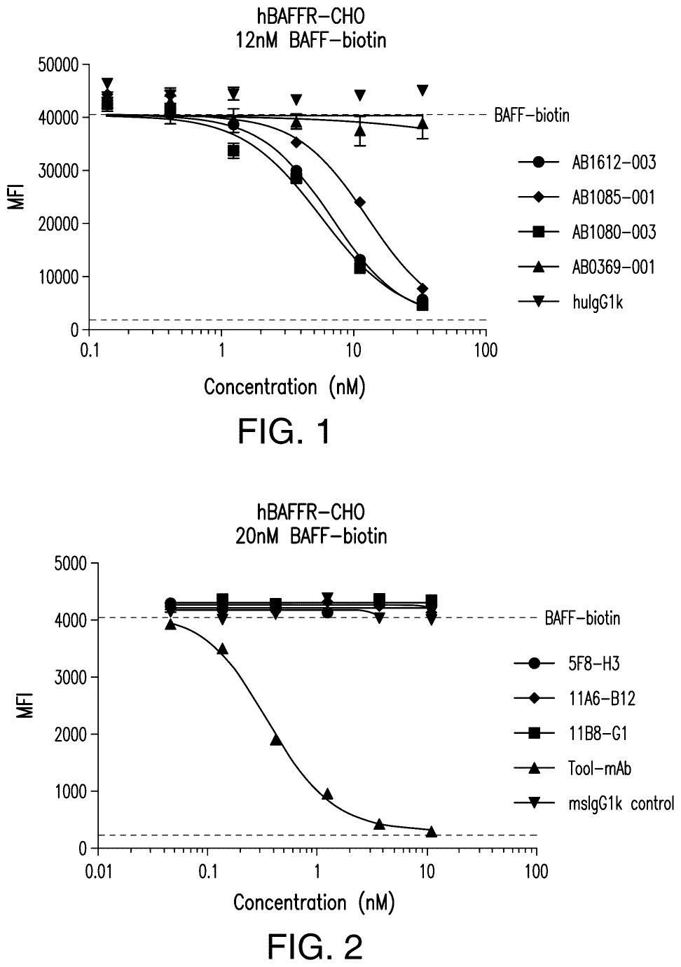

This Example describes two antibody discovery campaigns conducted to identify binders of BAFF-R. One binder was chosen for further development using yeast display technology, multiple rounds of affinity maturation (CDRH3 focused and CDRH1/CDRH2 focused), sequence liability correction, off-rate pressure optimization and site directed mutagenesis to improve biological properties. These studies identified binder AB1612/AB1424 as a binder displaying properties appropriate for a biologics drug candidate, and, importantly displaying the ability to inhibit BAFF-R-BAFF interaction (shown in FIG. 1).

### Recombinant Protein Immunization Methods

BAFF-R-specific antibodies were generated by immunizing four different strains of mice (H2L2, NZBW, BALB-C, and SJL/J) with hBAFF-R-hFc-His fusion protein. Based on antisera titers, a total of seven mice from across the four different strains were selected for hybridoma fusion. Splenocytes from a subset of mice from each immunization arm were reserved for immune library generation; however, only splenocytes from H2L2 mice were used for yeast display mAb discovery.

From five mice fusions (splenocytes from two mice were pooled for H2L2 fusion and splenocytes from two mice were pooled for SJL/J fusion), sixteen 96-well plates per hybridoma fusion were analyzed by specificity ELISA, in which binding to human and cynomolgus monkey BAFF-R-hFc-His and binding to irrelevant-hFc-His protein was compared. Supernatants from 33 BAFF-R positive and specific hybridomas were selected for further analysis. Supernatants were tested for binding to BAFF-R+ isogenic CHO cells, and 16 positive hybridomas were further subcloned. Supernatants from the subclones were analyzed by specificity ELISA as described above and 20 BAFF-R positive and specific subclones were tested for binding to BAFF-R+ cells. Nine subclone mAbs demonstrated strong binding to BAFF-R+ cells and were sequenced. Six unique sequences were obtained, and the corresponding mAbs were further analyzed for their ability to block BAFF-R-BAFF interactions in a cell-based assay.

Binding of biotinylated BAFF to BAFF-R+ CHO cells was tested in the absence or presence of the six BAFF-R specific mAbs or an isotype control mAb. Reduction of mean fluorescent intensity (MFI) in the presence of antibody suggested that a mAb inhibited engagement of BAFF binding to BAFF-R, thus was designated as blocking antibody. All clones tested did not inhibit BAFF binding to BAFF-R+ cells, and therefore, all six were termed non-blocking (FIG. 2).

### DNA Immunization Methods

DNA immunization of two groups of SWR/J mice cach was performed. One group was immunized with a full-length human BAFF-R cDNA construct, and the other with a mixture of full-length human BAFF-R and human BAFF-R extracellular domain cDNA constructs. Based on antisera titers, mice were pooled, and subsequently selected for single B cell sorting and another pool used for hybridoma fusion.

Single B cell sorting efforts yielded 44 human and cynomolgus monkey cross-reactive clones. These clones were sequenced, transiently expressed in 293 cells, and the specificity of the purified mAb was analyzed by flow cytometry in which binding to hBAFF-R⁺, cynoBAFF-R⁺ isogenic CHO cells and to parental cell line was compared. Eight binders were purified and further analyzed for their ability to bind to BAFF-R and to block BAFF-R-BAFF interactions. All eight clones were determined to be non-blocking and demonstrated weak affinity for hBAFF-R⁺ cancer cells.

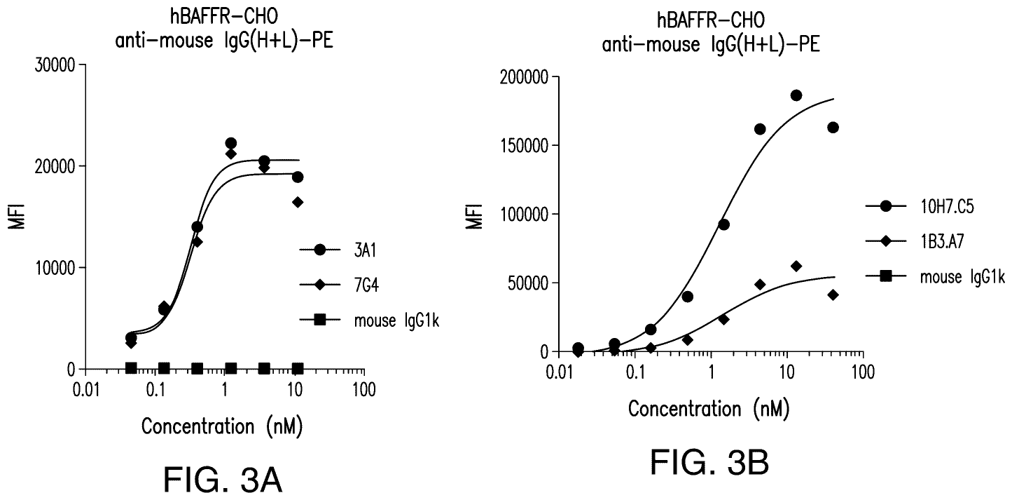

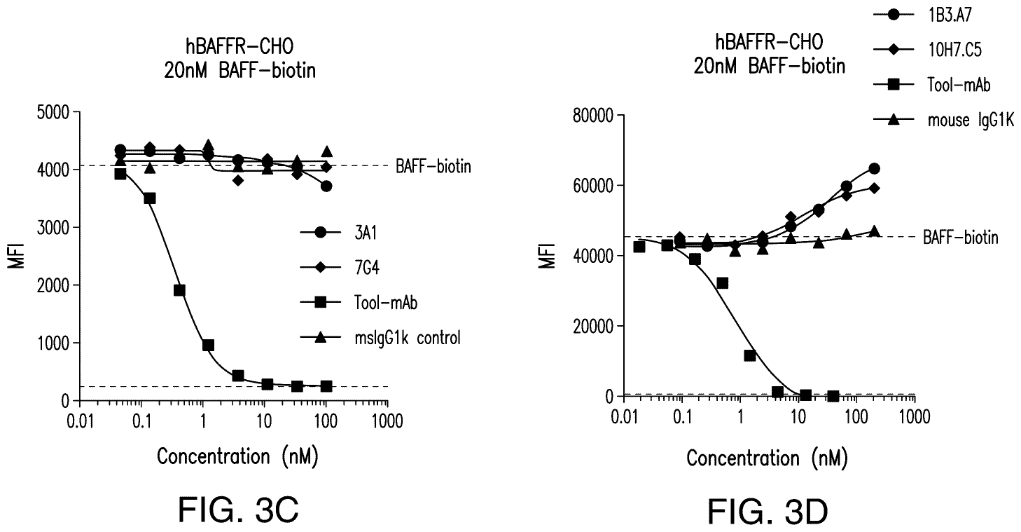

Specificity of clones obtained by the traditional hybridoma approach was analyzed by flow cytometry. The following assessment was performed: a) binding to cells expressing either full-length human BAFF-R or human BAFF-R extracellular domain was compared to binding to non-transfected parental cells; b) binding to hBAFF-R⁺ and cynoBAFF-R⁺ isogenic cell was compared to binding to the parental cells; c) binding to hBAFF-R⁺ cancer cells. 25 positive hybridoma fusions were identified and based on the binding intensity 14 hybridoma fusions were sequenced. Five unique sequences were obtained and analyzed for their ability to bind BAFF-R⁺ cells and block BAFF-R-BAFF interactions. Though all five clones were determined non-blocking clones (FIGS. 3A-3D), four out of five clones (clones 3A1, 1B3-A7, 7G4 and 10H7-C5) demonstrated good affinity for hBAFF-R.

BAFF-R Specific scFvs Discovered from Yeast Libraries

Yeast display was used to build scFv libraries from the splenocytes obtained from humanized H2L2 mice immunized with recombinant human hBAFF-R-hFc-His protein as described above. Three rounds of selection were carried out with biotinylated hBAFF-R-hFc-His at 5 nM. Individual yeast colonies were picked, sequenced, and sequences analyzed. Negative selection was performed to remove non-specific binders. Sequence convergence indicated the selection process was successful in enriching for binders and was therefore complete. Unique sequences were selected for further characterization. Three BAFF-R specific scFvs were discovered from one library (Table 4). However, these sequences were very similar to each other, and therefore only sequence 1129_A01 (also referred to as AB0369scFv) was selected for further study.

TABLE 4
CDR sequences of BAFF-R binders discovered from yeast library

| Clone ID | CDRH1 | CDRH2 | CDRH3 | CDRL1 | CDRL2 | CDRL3 |
|---|---|---|---|---|---|---|
| 1129_A01 | GFTFSSY | WYDGSN | RFTMLRGLIIEDYGMDV | RASQSISSYLN | AASSLQS | QQSYSTPLT |
| (AB0369 | (SEQ ID | (SEQ ID | (SEQ ID NO: 3) | (SEQ ID NO: 4) | (SEQ ID | (SEQ ID |
| scFv) | NO: 1) | NO: 2) | | | NO: 5) | NO: 6) |
| 1203_A01 | GFTFSSY | WYDGSN | RFTMLRGVFIEDYGMDV | RASQSVSSNLA | GASTRAT | QQSYSTPLT |
| | (SEQ ID | (SEQ ID | (SEQ ID NO: 7) | (SEQ ID NO: 8) | (SEQ ID | (SEQ ID |
| | NO: 1) | NO: 2) | | | NO: 9) | NO: 6) |
| 1203_A02 | GFTFSTY | WYDGSN | RNTMVRGVIIEDYGMDV | RASQSISSYLN | AASSLQS | QQSYSSPLT |
| | (SEQ ID | (SEQ ID | (SEQ ID NO: 11) | (SEQ ID NO: 4) | (SEQ ID | (SEQ ID |
| | NO: 10) | NO: 2) | | | NO: 5) | NO: 12) |

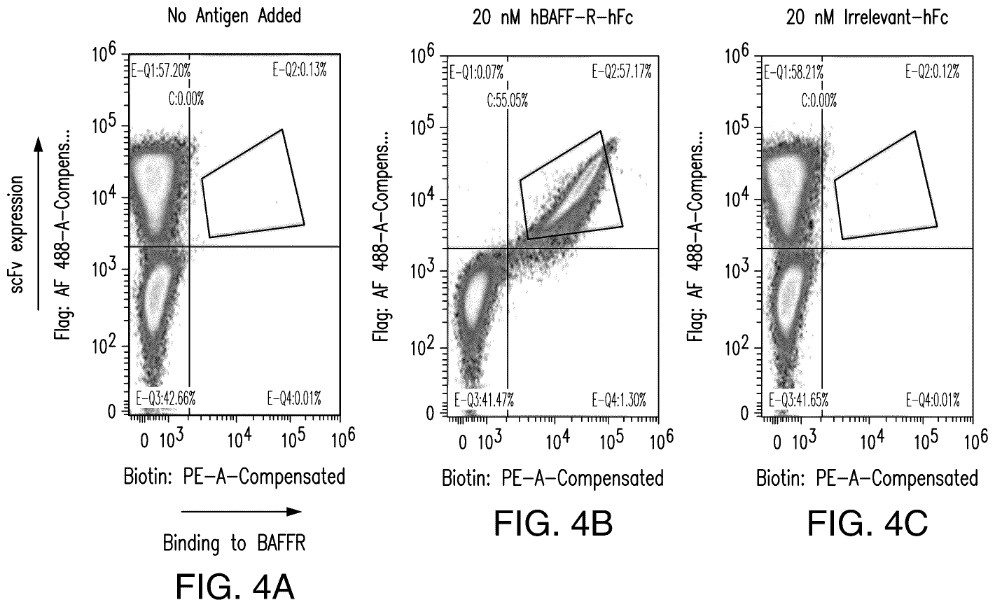

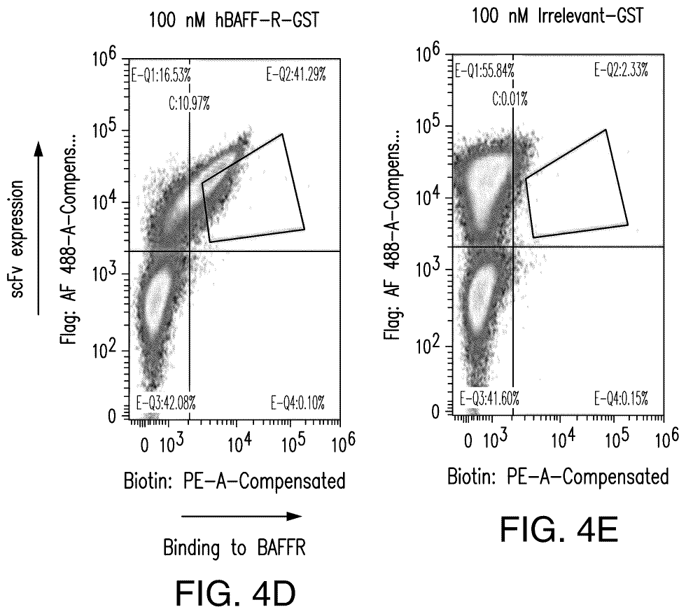

Flow cytometry was used to assess specificity of binding of AB0369scFv to hBAFF-R-hFc-His, hBAFF-R-GST-His, and negative control proteins with hFc tag or GST tag while displayed on yeast. AB0369scFv demonstrated medium to weak affinity towards hBAFF-R; however, it did not show binding to the negative control, thus suggesting high specificity for BAFF-R (FIGS. 4A-4E).

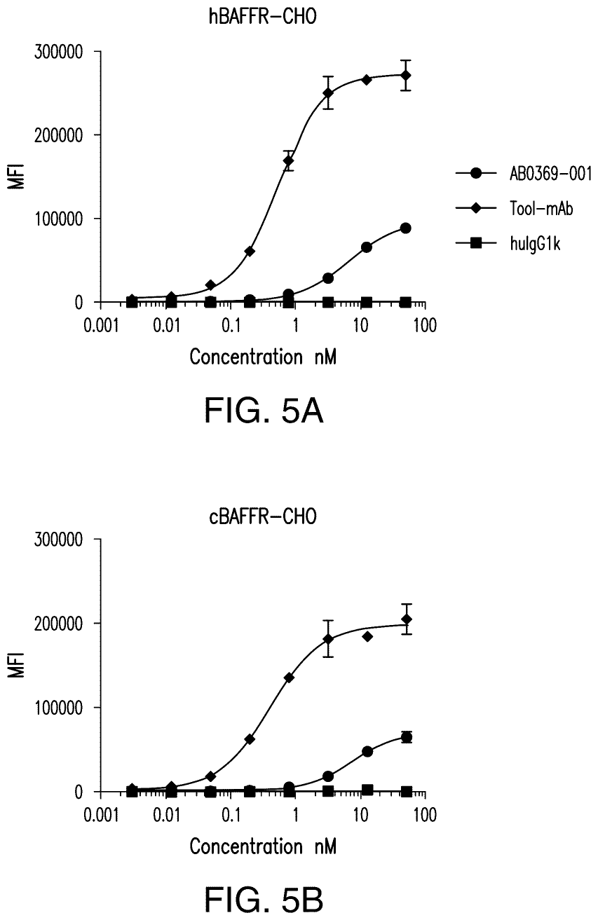

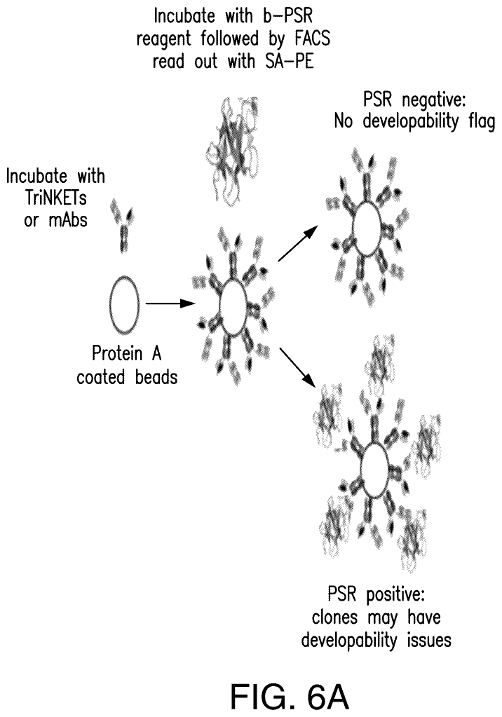

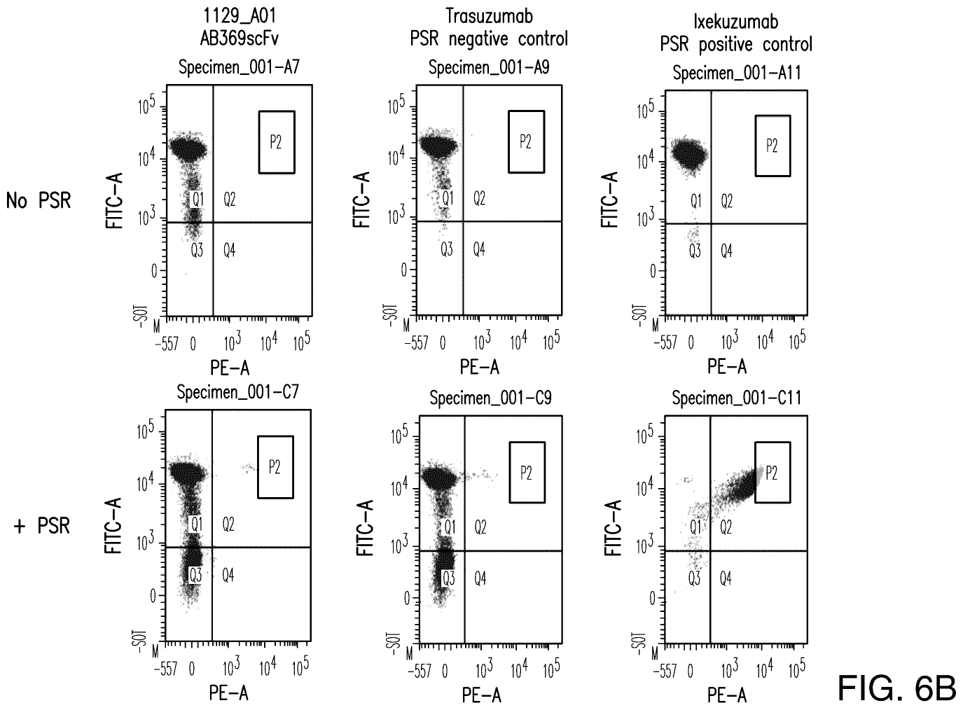

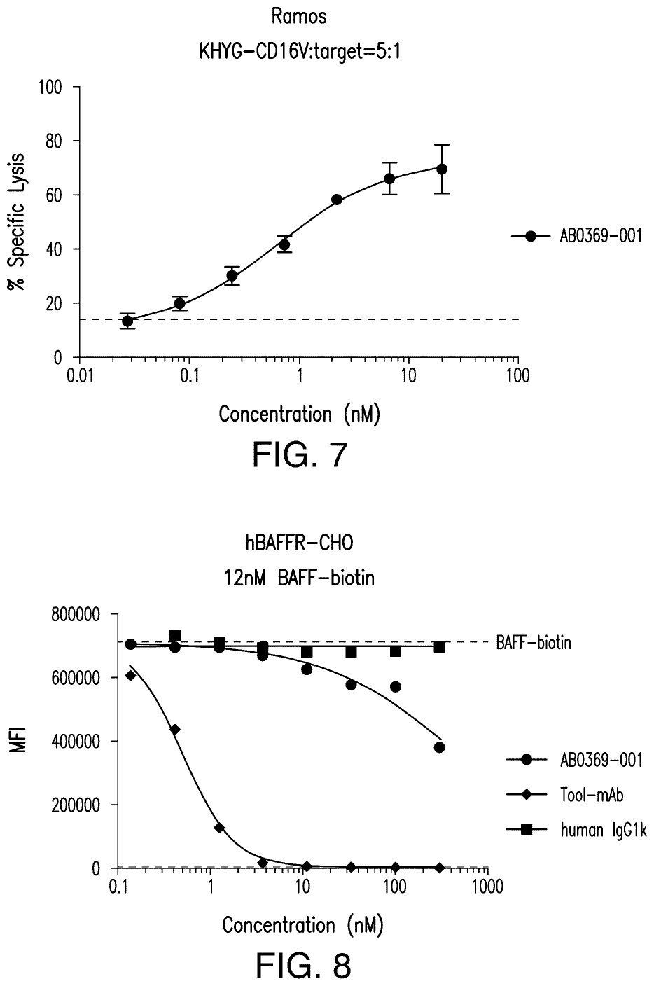

1129_A01 (AB0369 scFv) was converted into a multispecific binding protein comprising the scFv, and two non-BAFF-R binders, to yield AB0369. AB0369 was further analyzed for its abilities to bind to human (hBAFF-R-CHO) BAFF-R⁺ cells (FIG. 5A) and cynomolgus monkey (cBAFF-R-CHO) BAFF-R⁺ cells (FIG. 5B), lack non-specific interactions by polyspecificity reagent (PSR) assay (FIG. 6A, FIG. 6B), lyse BAFF-R⁺ Ramos cancer cells (FIG. 7 and Table 5) and block BAFF-BAFF-R interactions (FIG. 8). AB0369 bound to both human and cynomolgus monkey BAFF-R on the surface of isogenic CHO cells, and BAFF-R binding was with EC₅₀ about 10 nM, making it a good choice for further development.

TABLE 5
Potency of AB0369 in KHYG-1-CD16aV cytotoxicity assay.

| Molecule | EC₅₀ (nM) | Max Lysis (%) |
|---|---|---|
| AB0369-001 | 0.6 | 73 |

The ability of AB0369 to block BAFF-R-BAFF interactions was tested in a cell-based blocking assay. Briefly, CHO cells expressing human BAFF-R were harvested, washed in cold FACS buffer, and seeded at a density of 100,000 cells per well. Test articles were diluted in FACS buffer, and 50 μL of diluted multispecific binding protein or mAb was added to cells, incubated on ice for 60 minutes, then washed with FACS buffer. 12 nM BAFF-biotin was diluted into FACS buffer, and 100 μL was added per well, incubated for 60 minutes on ice, then washed with FACS buffer. Cells were incubated with 100 μL of 1:200 streptavidin-PE diluted in FACS buffer and incubated on ice for 30 minutes then washed with FACS buffer. Cells were then incubated in 100 μL of 1:1,000 dilution of live/dead dye in PBS for 15 minutes, then washed with FACS buffer, and fixed. After incubation, cells were washed with FACS buffer and resuspended in FACS buffer for analysis with flow cytometry. Median fluorescent intensity (MFI) of each sample and the secondary-only control was calculated. Maximum MFI was calculated as BAFF-biotin alone, and minimum MFI was calculated as streptavidin-Phycoerythrin alone. Data were fit to a four-parameter non-linear regression curve using GraphPad Prism.

These studies revealed that AB0369 was able to partially block BAFF-R-BAFF interactions. However, the blocking was significantly less potent than the ianalumab-based benchmark control, which does not contain antibody-dependent cellular cytotoxicity enhancing mutations unlike the parent antibody, presumably due to the low affinity of AB0369 (FIG. 8 and Table 6). As the AB0369 scFv was the only blocking antibody identified from all discovery efforts described above, it underwent further development by affinity maturation of CDRH3 and CDRH1/CDRH2, as well as further amino acid changes to facilitate protein production and stability.

TABLE 6
Summary of AB0369 and benchmark mAb blocking
of BAFF binding to cellular BAFF-R.

| Molecule | IC₅₀ (nM) | Minimum (MFI) |
|---|---|---|
| AB0369-001 | 488 | 38,180 |
| Ianalumab-based tool mAb | 0.5 | 224 |
| Human IgG1k | N/A | 68,050 |

### Affinity Maturation of AB0369

### CDRH3 Focused Randomized Affinity Maturation

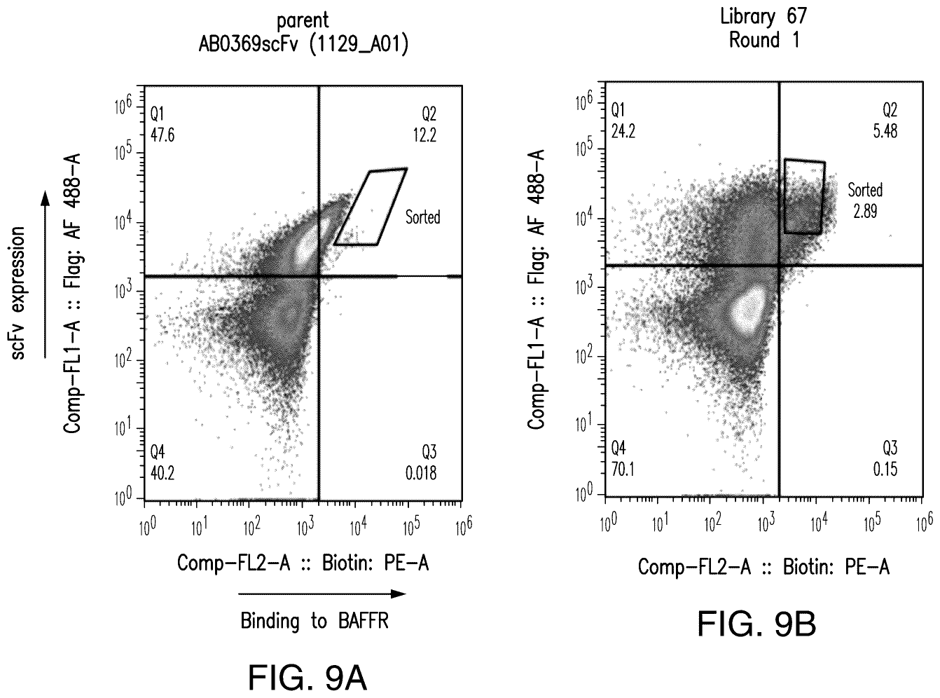

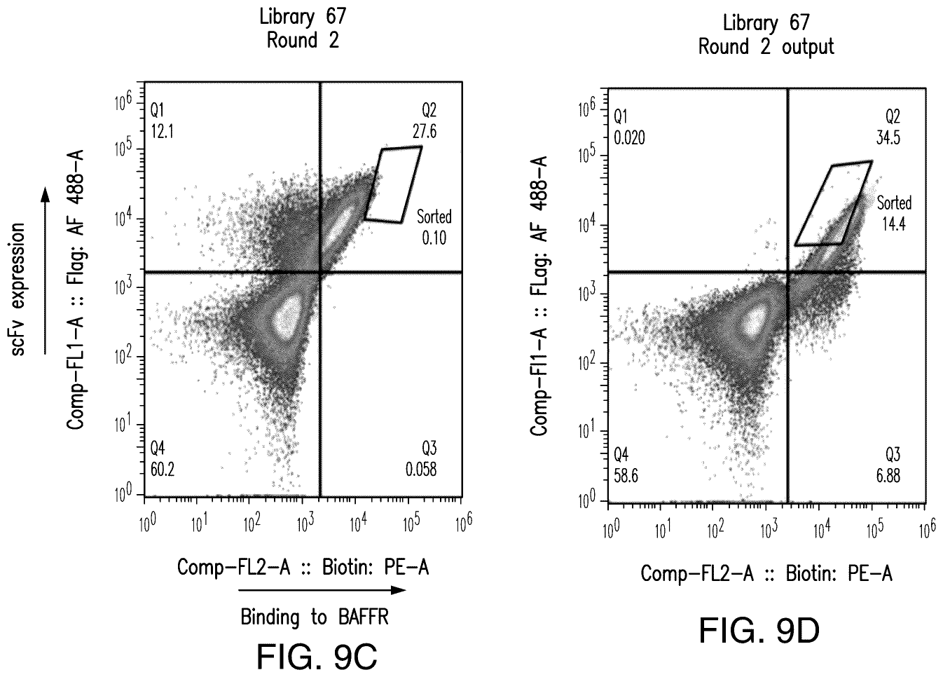

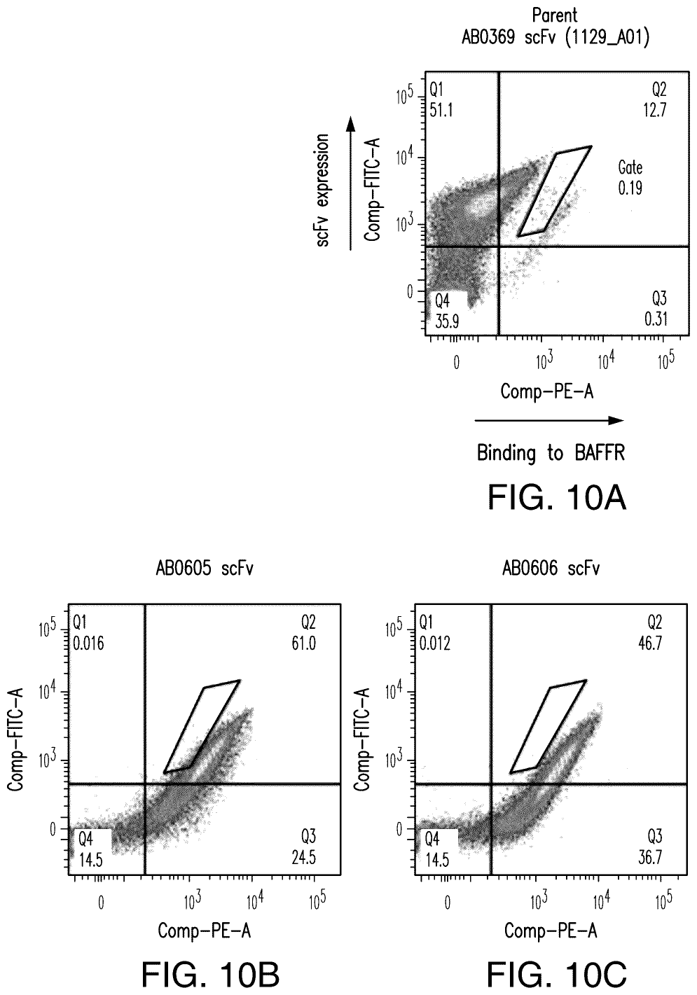

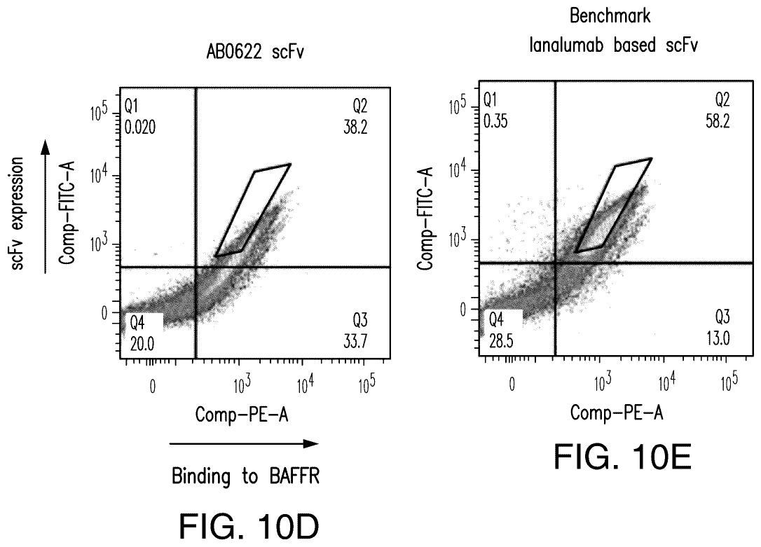

As described above, AB0369 demonstrated specific binding to BAFF-R expressing cells. To search for variants with improved binding affinities, a yeast display affinity maturation library was created by mutating the CDRH3 residue (RFTMLRGLIIEDYGMDV (SEQ ID NO:3) of AB0369. To enrich for scFvs that have higher affinity towards hBAFF-R, two rounds of selection were carried out with biotinylated hBAFF-R-hFc-His at 1 nM (FIGS. 9A-9D). The affinities between the parental clone AB0369 and representative individual library clones were compared. Three rounds of FACS sorting resulted in nine clones that contained one or two amino acid differences as compared to the parental clone (RFTMLRGWYIEDYGMDV (SEQ ID NO:14); RFTMLRGQYIEDYGMDV (SEQ ID NO:13); RFTMLRGWIIEDYGMDV (SEQ ID NO:15)) and exhibited higher binding affinity for hBAFF-R than the parental clone and parental-derived scFv, using an ianalumab-based scFv used as a benchmark control (FIGS. 10A-10E).

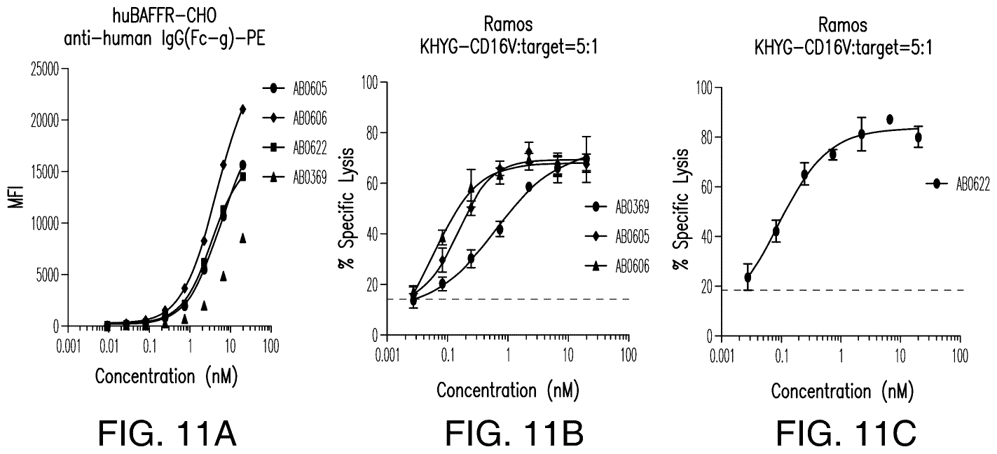

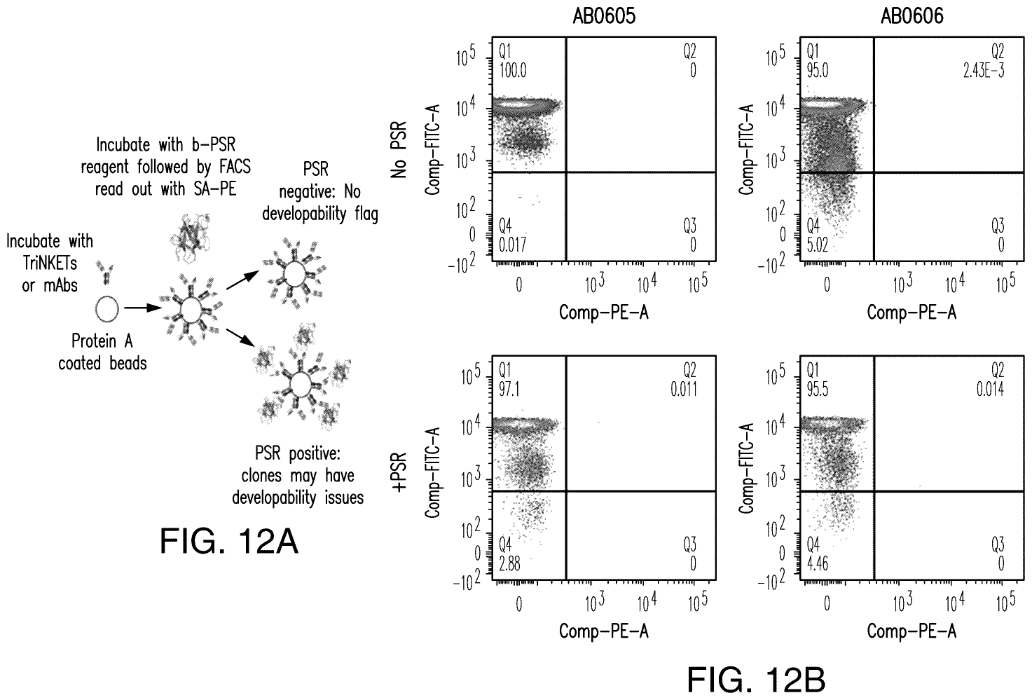

The scFvs with highest hBAFF-R binding affinity were converted into multispecific binding proteins comprising the scFv, and two non-BAFF-R binders, expressed in Expi293 cells, and further analyzed for their ability to bind to BAFF-R expressing cells (FIG. 11A) and ability to lyse BAFF-R expressing Ramos cancer cells (FIG. 11B, FIG. 11C). All multispecific binding proteins scored negatively in a poly-specificity assay, suggesting that the improved binding affinity was BAFF-R specific (FIGS. 12A-12B). Further studies demonstrated greater than three-fold improvement in BAFF-R binding, which translated into six to ten-fold improvement in potency as measured by EC₅₀ (Table 7). Maximum lysis remained unchanged, suggesting that the improvement in BAFF-R binding affinity was the key driver of this improvement in potency.

TABLE 7
Summary of cell binding and cytolysis demonstrated
by multispecific binding proteins based on HCDR3 affinity
matured variants compared to parental AB0369.

| | | KHYG-A-CD16aV |
|---|---|---|
| | BAFF-R-CHO | mediated Cytolysis |
| | cell binding | of Ramos cells |
| Molecule | EC₅₀ (nM) | EC₅₀ (nM) |
| AB0605-001 | 5.72 | 0.15 |
| AB0606-001 | 4.50 | 0.06 |
| AB0622-001 | 3.45 | 0.09 |
| AB0369-001 | >10 | 0.64 |

### CDRH1 and CDRH2 Focused Combinatory Affinity Maturation

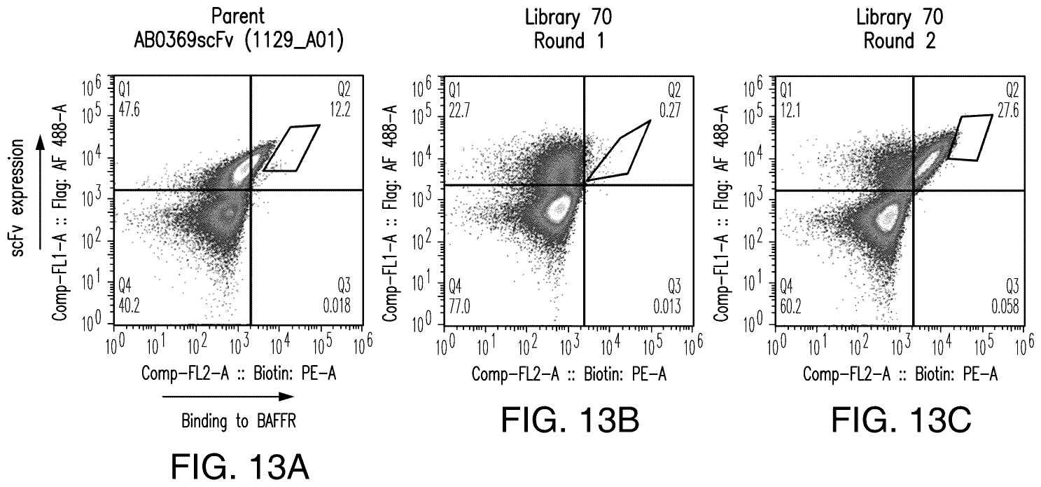

Outcomes from the CDRH3 focused affinity maturation studies demonstrated an improvement in affinity, and further improvement was highly desirable. Thus, the CDRH1 and CDRH2 sequences were selected for affinity maturation (CDRH1: GFTFSSY (SEQ ID NO:1) and CDRH2: WYDGSN (SEQ ID NO:2)) using the matured CDRH3 backbone. The goal was to engineer and select binders with improved affinity over the parental clone (AB0369 scFv) or the CDRH3 optimized variants described above. This created a library with a randomized CDRH1 and CDRH2 while retaining an optimized CDRH3. Two rounds of FACS were performed to enrich for high affinity binders (FIGS. 13A-13C).

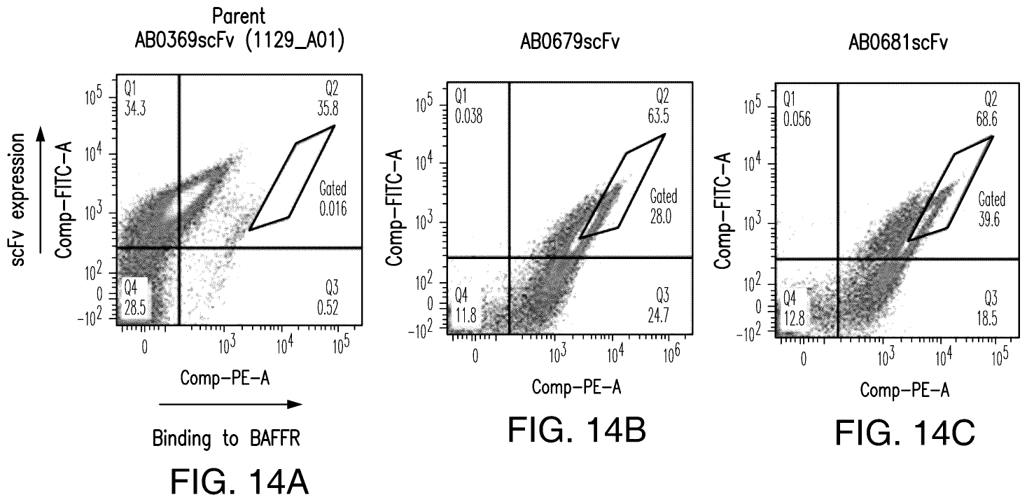

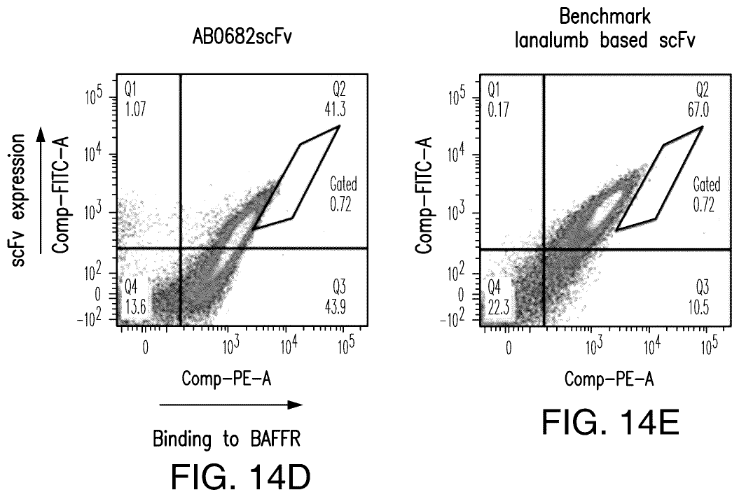

After FACS, 24 clones were identified. It was observed that several clones with changes in CDRH1 (RFTMLRGWYIEDYGMDV (SEQ ID NO: 14); RFTMLRGQYIEDYGMDV (SEQ ID NO:13); RFTMLRGWIIEDYGMDV(SEQ ID NO:15)) on the optimized CDRH3 backbone showed a significant improvement in hBAFF-R affinity compared to parental AB0369scFv (1129_A01) (FIGS. 14A-14D) or to an ianalumab-based scFv benchmark control (FIG. 14E).

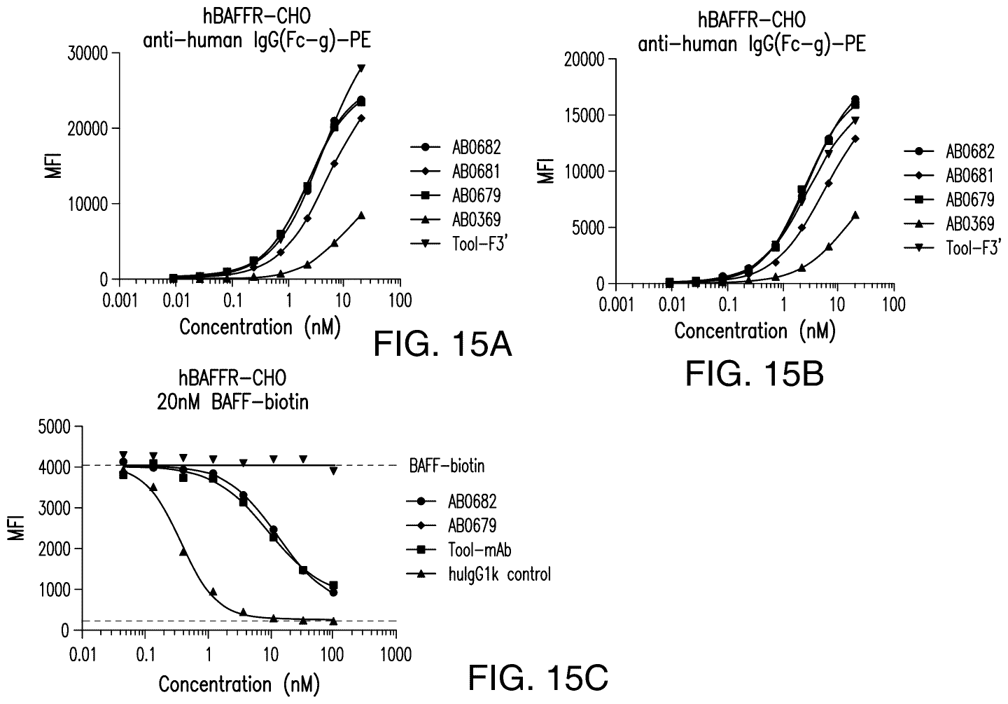

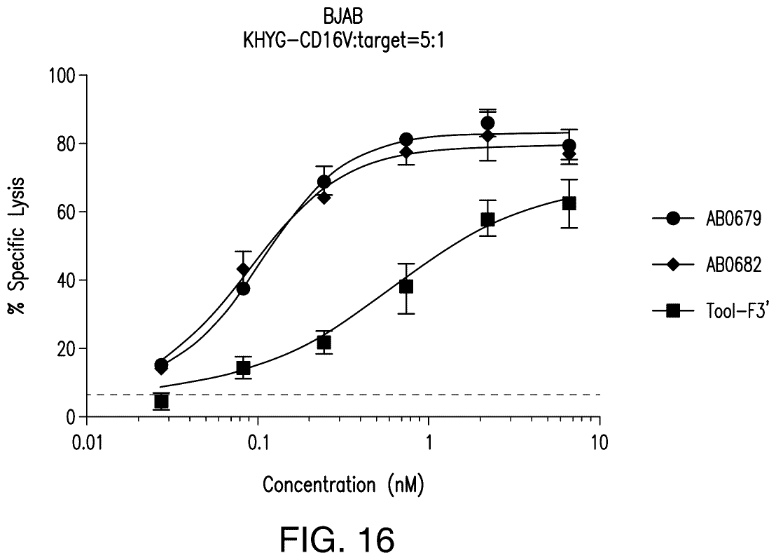

The scFvs with the highest hBAFF-R binding affinities were converted into multispecific binding proteins comprising the scFv, and two non-BAFF-R binders, expressed in Expi293 cells, and further analyzed for their ability to bind to human BAFF-R expressing cells (FIG. 15A), to bind to cynomolgus BAFF-R⁺ cells (FIG. 15B), and to inhibit BAFF-R-BAFF interactions (FIG. 15C and Table 8). Tested multispecific binding proteins showed improvement in all three of these criteria and demonstrated efficient killing of BAFF-R⁺ BJAB cells in a KHYG-1-CD16a mediated cytotoxicity assay (FIG. 16, Table 9).

TABLE 8
Summary of BAFF-R cell binding and BAFF-R-BAFF blocking
demonstrated by multispecific binding proteins
based on CDRH1 and CDRH2 affinity maturation

| | Human BAFF-R | Cyno BAFF-R | BAFF-BAFF-R |
|---|---|---|---|
| | cell binding | cell binding | blocking |
| Molecule | EC₅₀ (nM) | EC₅₀ (nM) | IC₅₀ (nM) |
| AB0682-001 | 2.4 | 3.1 | 12.6 |
| AB0681-001 | 5.1 | 5.6 | — |
| AB0679-001 | 2.3 | 2.6 | 9.4 |
| AB0369-001 | >10 | >15 | — |
| Tool-F3′ | 4.3 | 2.9 | — |
| Tool-mAb | — | — | 0.38 |

TABLE 9
Potency of representative multispecific binding
proteins based on CDRH1 and CDRH2 affinity maturation
in a KHYG-1-CD16V cytolysis assay.

| Molecule | EC50 (nM) | Max Lysis (%) |
|---|---|---|
| AB0679-001 | 0.11 | 83 |
| AB0682-001 | 0.09 | 79 |
| Tool-F3′ | 0.61 | 69 |

### Remediation of Potential Sequence Liabilities

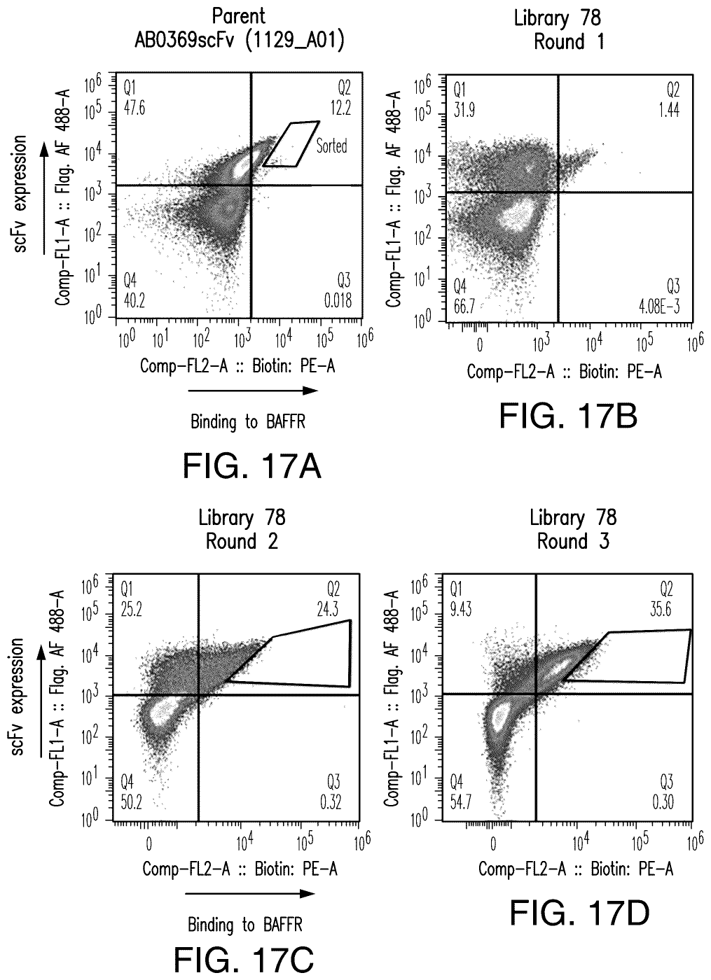

Because the affinity matured clones contained amino acids in their CDRs that could negatively impact protein expression, stability, or immunogenicity, additional libraries were constructed to select clones without these amino acids. Three rounds of selection were performed with 1 nM biotinylated hBAFF-R-hFc-His protein leading to enrichment of high affinity binders (FIGS. 17A-17D). 23 binders were identified altogether, 12 of which were predicted to be free of undesirable amino acids (“liability-corrected”).

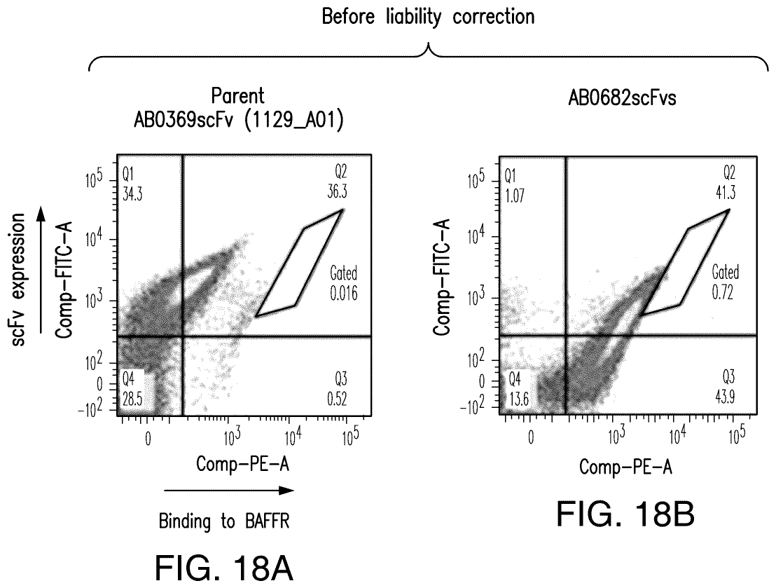

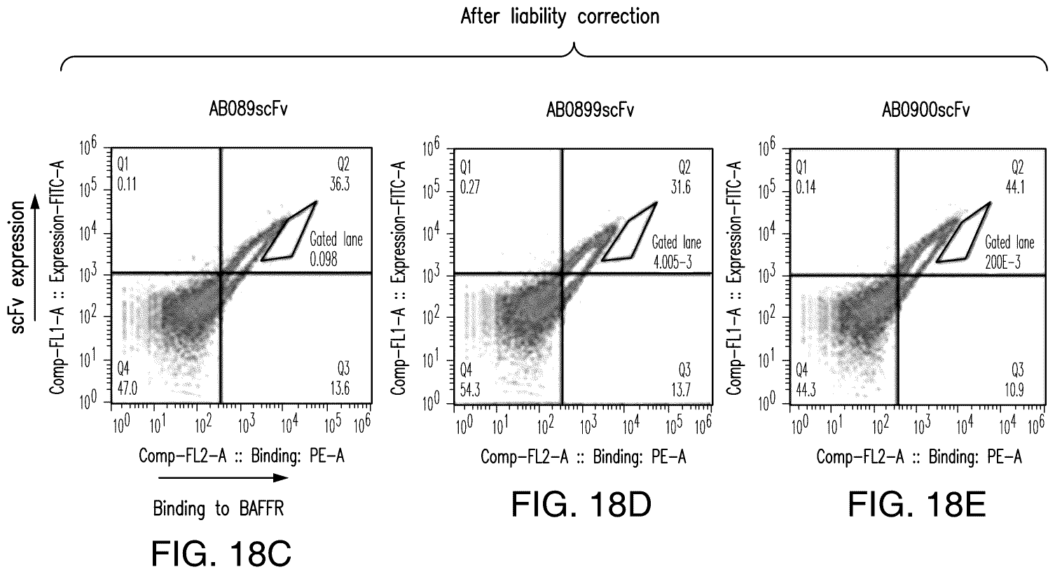

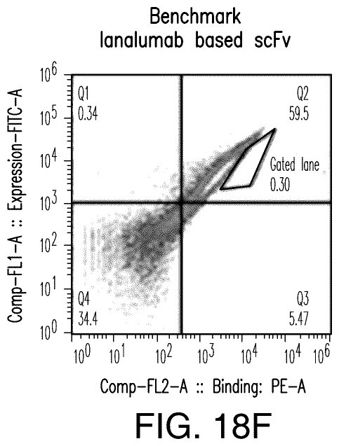

Preferred clones lacking potential sequence liabilities from these libraries included AB0898, (the liability corrected version of AB0682 described above), AB0899, and AB0900, which were successfully identified and tested for their binding to hBAFF-R while displayed on yeast. All clones showed higher affinity towards hBAFF-R than the parent, AB0369scFv (FIGS. 18A-18F).

### Characterization of Liability-Corrected Multispecific Binding Proteins

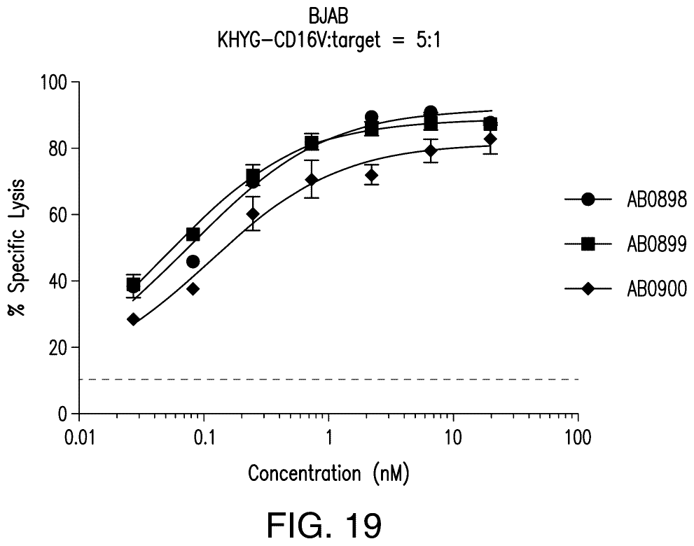

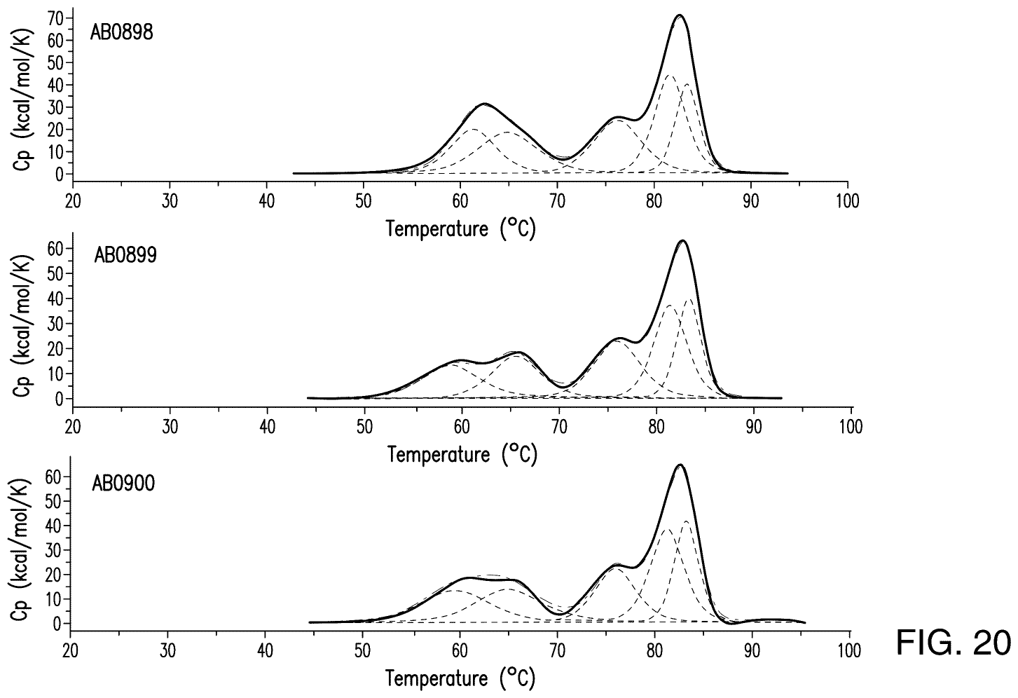

Three of the liability-corrected clones were converted into multispecific binding proteins comprising the scFv, and two non-BAFF-R binders, expressed in Expi293 cells, purified by a two-step purification process and characterized by size-exclusion chromatography (SEC), differential scanning calorimetry (DSC), binding to BAFF-R-expressing cells, and ability to lyse BJAB cells in a KHYG-1-CD16aV mediated cytotoxicity assay. Characterization of these clones is summarized in Table 10 and demonstrates that the liability correction was successful. No negative effect on cell binding was observed and all three clones demonstrated potent killing of BAFF-R-expressing tumor cells (FIG. 19). However, the thermostability of the molecules was Tm₁>65° C., as shown in FIG. 20.

TABLE 10
Summary of characterization of multispecific binding proteins
expressing sequence liability corrected BAFF-R binders.

| | SEC, | DSC, | hBAFF-R⁺ | KHYG-A-CD16aV mediated |
|---|---|---|---|---|
| Test | monomer | Tₘ₁ | cell binding | Cytolysis of BJAB cells, |
| Article | (%) | (° C.) | EC₅₀ (nM) | EC₅₀ (nM) |
| AB0898 | 86.9 | 61.23 | 4.3 | 0.08 |
| AB0899 | 96.5 | 58.79 | 4.3 | 0.06 |
| AB0900 | 90.0 | 59.41 | 11.7 | 0.11 |

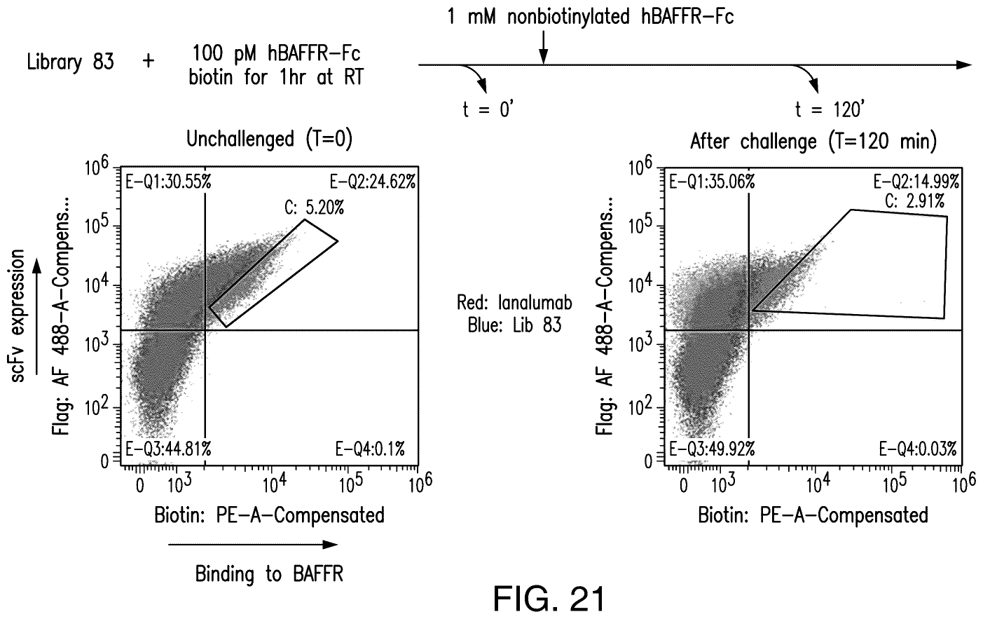

As described above, replacement of the potential sequence liability residues with certain amino acids in CDRs had minimal effect on binding affinity; however, BAFF-R expressing cell binding and thermostability data suggested that further improvement was desirable. Thus, the CDRH1 and CDRH2 sequences (CDRH1: GFTFSSY (SEQ ID NO:1) and CDRH2: WYDGSN (SEQ ID NO:2)) were affinity matured into the liability-corrected CDRH3 backbone, and off-rate pressure was applied to select high affinity clones. Briefly, clones were preincubated with biotinylated hBAFF-R-hFc-His at 100 pM concentration, and then challenged with 1 μM non-biotinylated hBAFF-R-hFc-His for 2 hours. Yeast displaying anti-BAFF-R scFvs that remained bound to biotinylated hBAFF-R-hFc-His were sorted and the process was repeated three times to enrich for high affinity binders with slower off-rate. As shown in FIG. 21, clones remained bound to biotinylated hBAFF-R-hFc-His even after the off-rate pressure challenge, whereas the ianalumab-based scFv benchmark control lost binding to biotinylated hBAFF-R-hFc-His under these conditions, suggesting a slower dissociation rate.

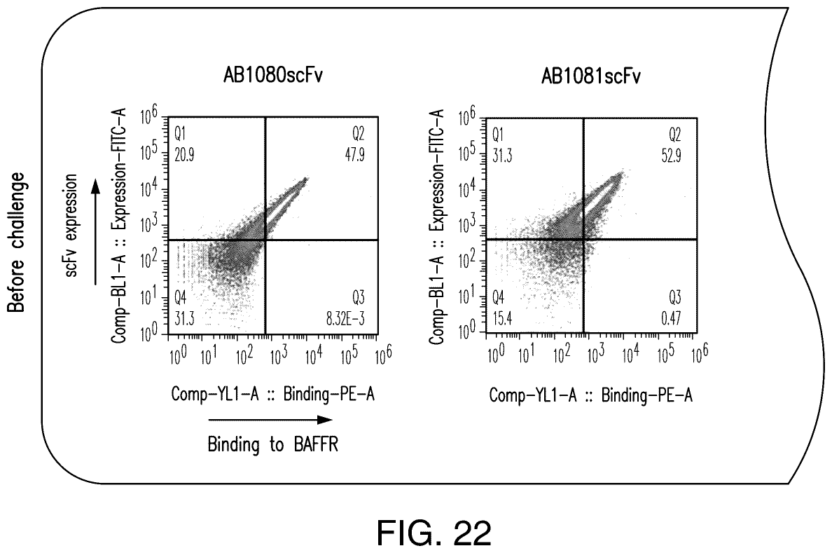

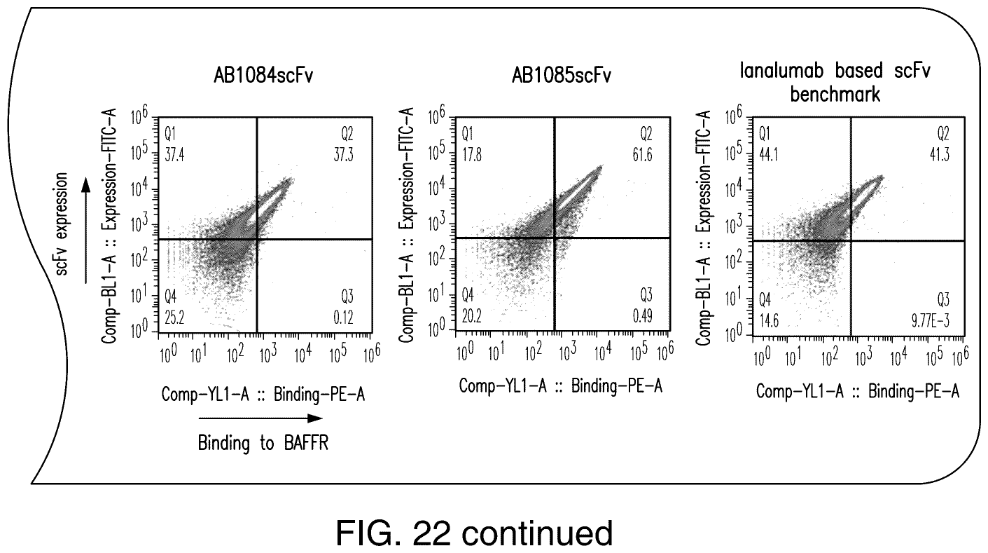

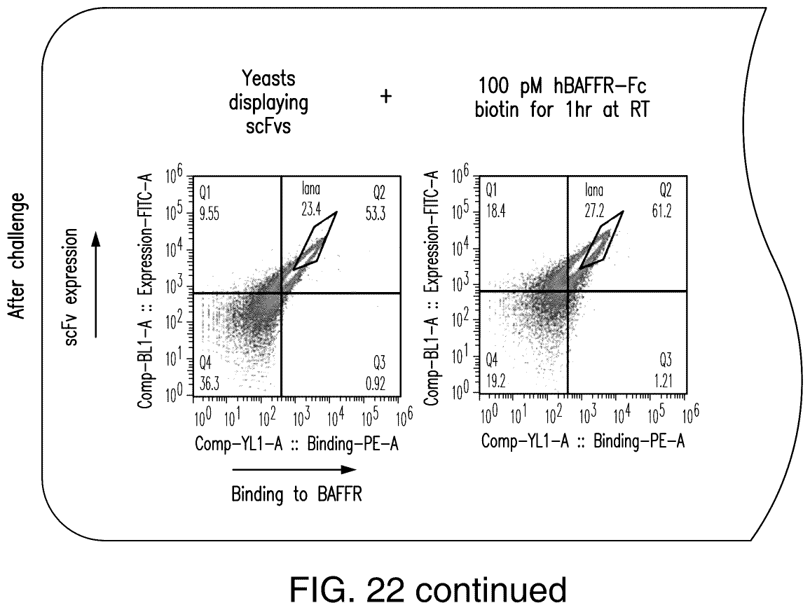

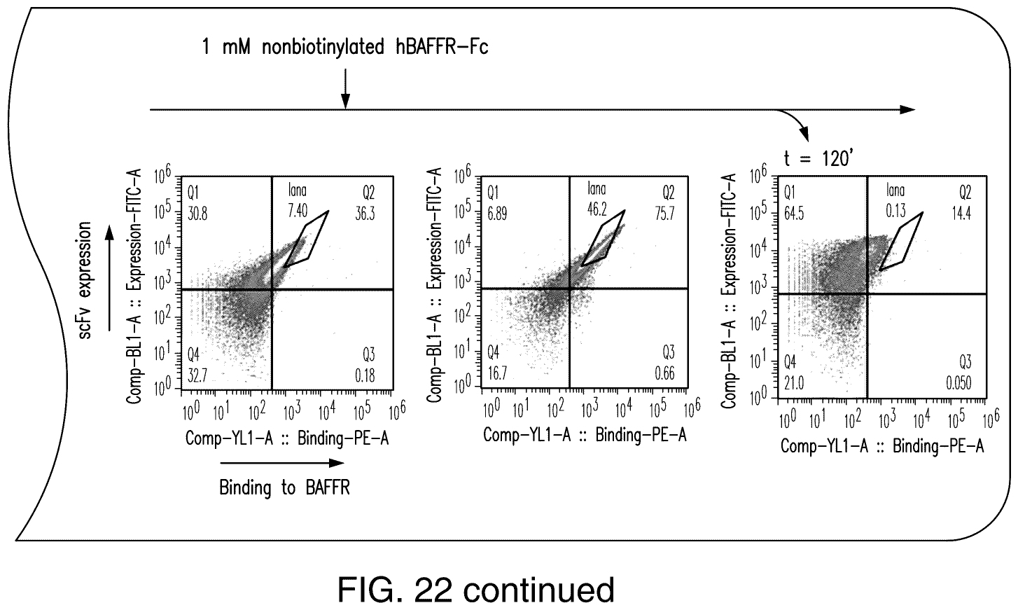

Analysis of individual clones demonstrated high affinity towards hBAFF-R-hFc-His (FIG. 22) and importantly, the clones remained bound to biotinylated hBAFF-R-hFc-His. Notably, ianalumab-based benchmark scFv exhibited loss of binding to biotinylated hBAFF-R-hFc-His after the challenge (FIG. 22). Several of the clones were eliminated from further consideration because they contained additional undesirable amnio acids or properties. Sequences of selected clones from the above studies are shown in Table 11.

TABLE 11
CDR sequences of selected clones.

| Sequence | CDRH1 | CDRH2 | CDRH3 |
|---|---|---|---|
| AB1080scFv | GFTFSSY | WYDASN | RFTHLRGWYIEDYGLDV |
| | (SEQ ID NO: 1) | (SEQ ID NO: 23) | (SEQ ID NO: 27) |
| AB1081scFv | GFAFSSY | WYDESN | RFTNLRGWIIEDYGLDV |
| | (SEQ ID NO: 28) | (SEQ ID NO: 29) | (SEQ ID NO: 30) |
| AB1084scFv | GFTFSMY | WYDASN | RFTRLRGWYIEDYGLDV |
| | (SEQ ID NO: 31) | (SEQ ID NO: 23) | (SEQ ID NO: 32) |
| AB1085scFv | GFTFGSY | WYDGSN | RFTHLRGQYIEDYGMDV |
| | (SEQ ID NO: 33) | (SEQ ID NO: 2) | (SEQ ID NO: 34) |

Potential sequence liabilities are bold-underlined and residues demonstrating diversity between clones are bolded.

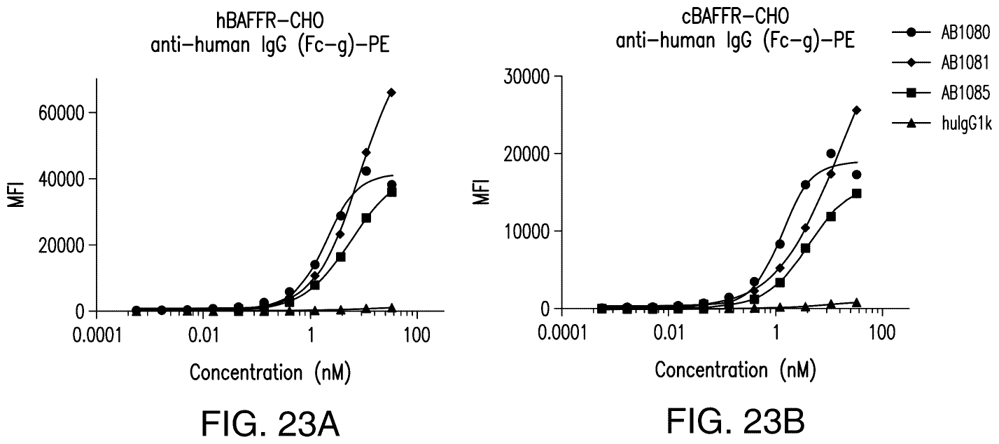

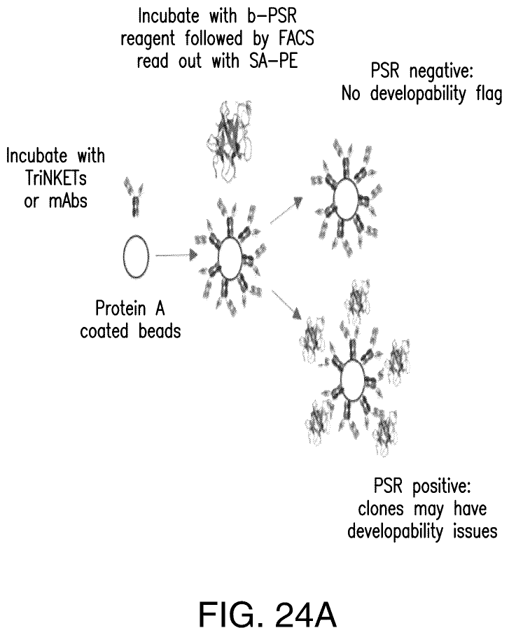

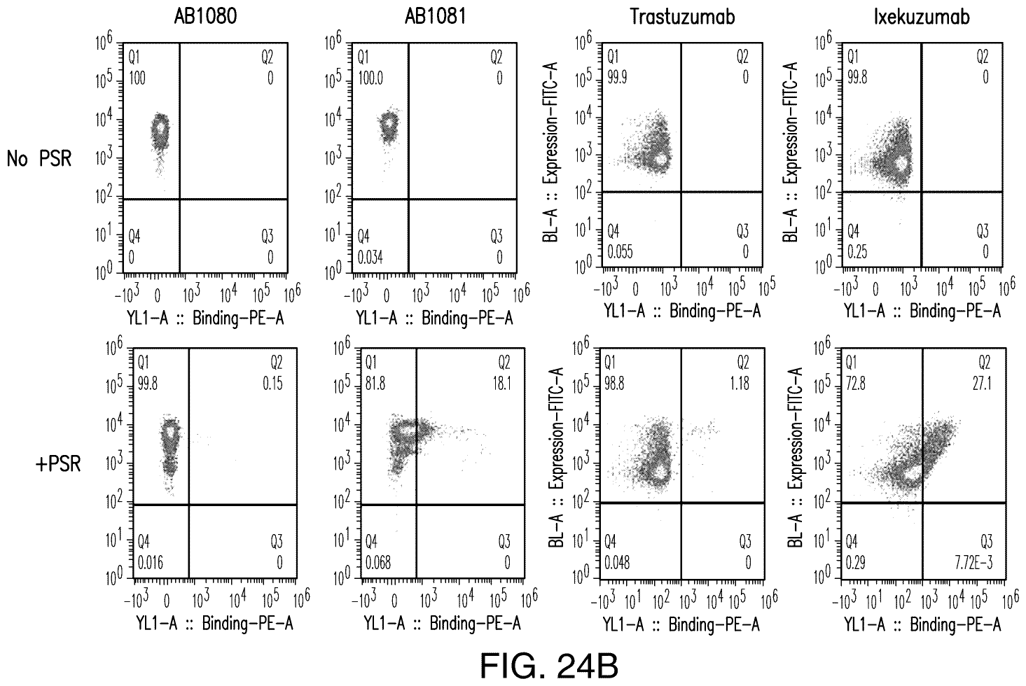

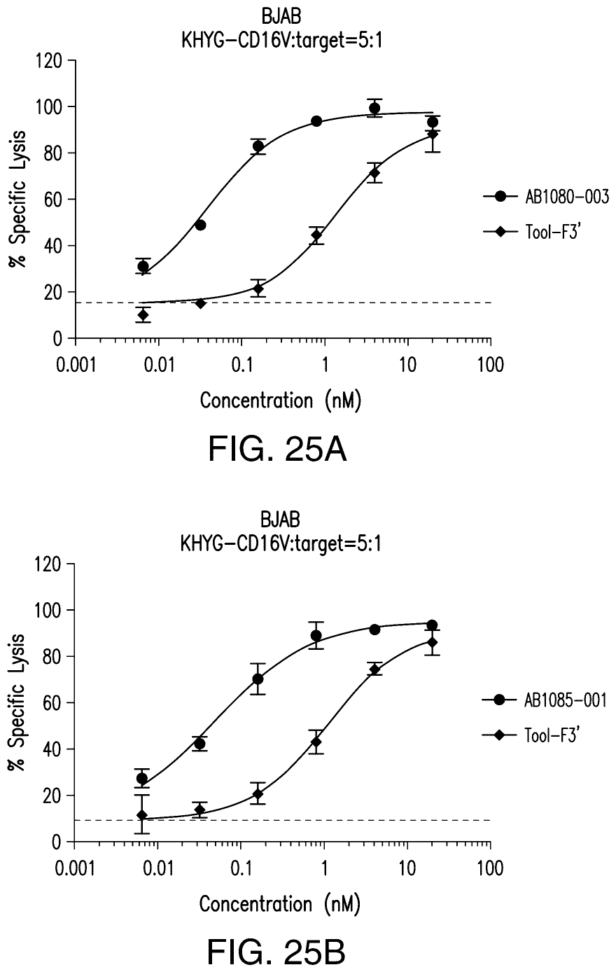

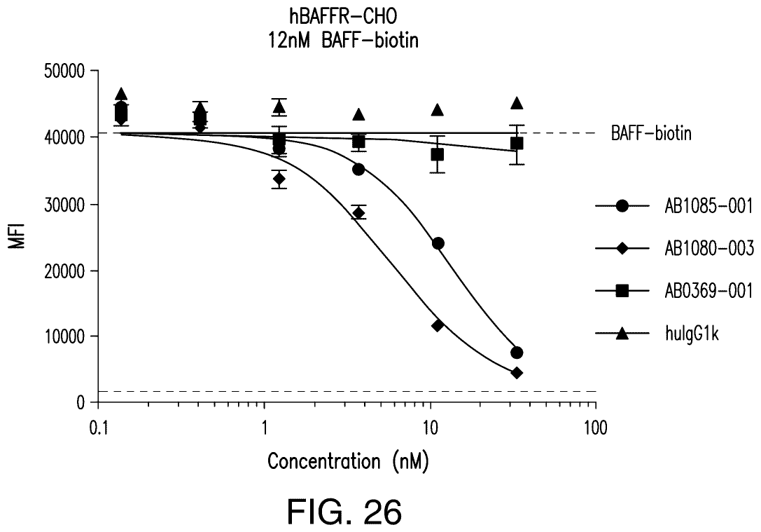

Selected clones from the off rate challenge studies described above were produced as multispecific binding proteins comprising an scFv of the respective binders, and two non-BAFF-R binders, expressed in Expi293 cells, and characterized by binding to hBAFF-R expressing cells and cynomolgus BAFF-R expressing cells, ability to lyse BAFF-R expressing cancer cells in a KHYG-1-CD16aV mediated cytotoxicity assay, ability to block BAFF-BAFF-R interactions, thermostability (differential scanning fluorimetry, DSF) and hydrophobicity (HIC) (results are summarized in Table 12). Binding affinity of AB1080, AB1081, and AB1085 to BAFF-R⁺ cells was improved as compared to the parental clones (FIGS. 23A-23B as compared to Table 12). Additionally, binding affinity to cynoBAFF-R was similar to binding affinity to hBAFF-R (FIGS. 23A-23B). Lack of polyspecificity was confirmed by a PSR assay (FIGS. 24A-24B). AB1084 was removed from further study, due to long retention time on HIC and subsequent potential for higher aggregation propensity. Improved multispecific binding proteins demonstrated vastly higher potency than the multispecific binding protein based on the ianalumab sequence (FIGS. 25A-25B). In addition, a greater than ten-fold improvement in potency was observed as compared to the original AB0369 multispecific binding protein. Importantly, the ability to block BAFF-BAFF-R binding was significantly improved as compared to the parental AB0369 multispecific binding protein (FIG. 26).

TABLE 12
Summary of characterization of selected
multispecific binding proteins.

| | hBAFF-R⁺ | cBAFF_R⁺ | | HIC, | DSF, |
|---|---|---|---|---|---|
| Test | Cell binding | Cell binding | Cytolysis, | retention | Tm1 |
| Article | EC₅₀ (nM) | EC₅₀ (nM) | EC₅₀ (nM) | (min) | (° C.) |
| AB1080-002 | 1.97 | 1.36 | 0.03 | 11.40 | 66.3 |
| AB1081-002 | >8 | >14 | 0.05 | 11.45 | 65.9 |
| AB1084-001 | — | — | 0.06 | 11.79 | 67.5 |
| AB1085-001 | 5.78 | 4.34 | 0.08 | 9.55 | 68.1 |

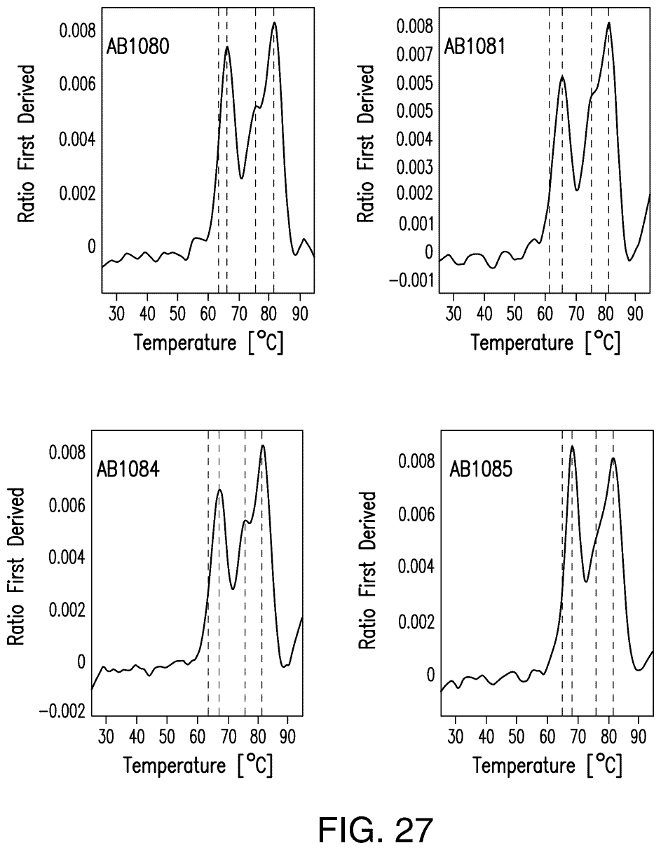

These multispecific binding proteins satisfied criteria for acceptable thermostability as compared to controls adalimumab (Humira) and pembrolizumab (Keytruda) (FIG. 27). HIC chromatograms revealed that AB1080 and AB 1081 had retention times of 11.4 and 11.5 min, respectively. AB1085 demonstrated a retention time of 9.5 minutes, which is at the lower edge among approved and late-stage therapeutic antibodies, suggesting very favorable hydrophobic behavior (FIG. 27).

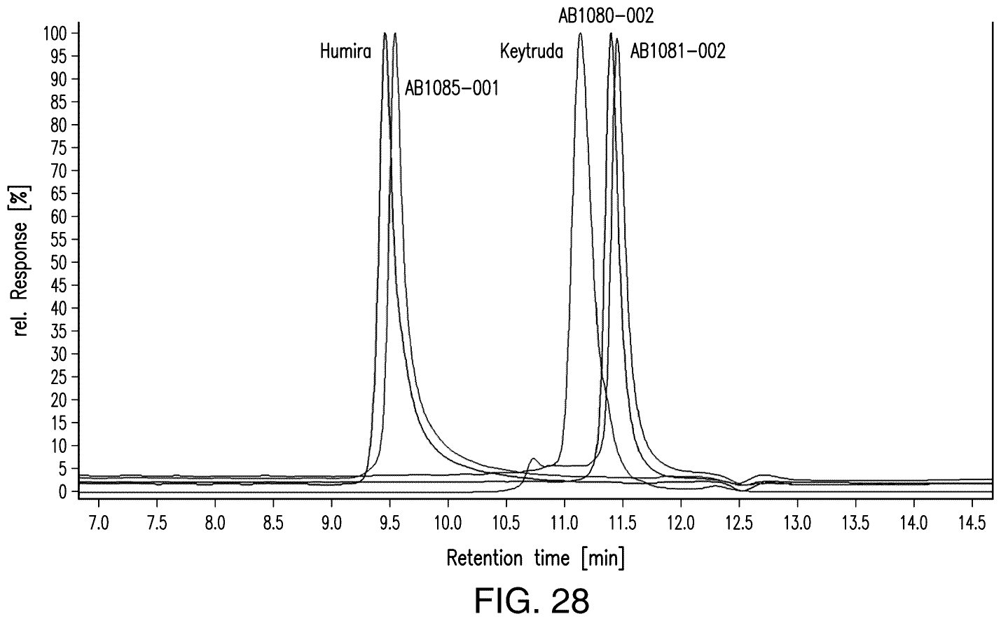

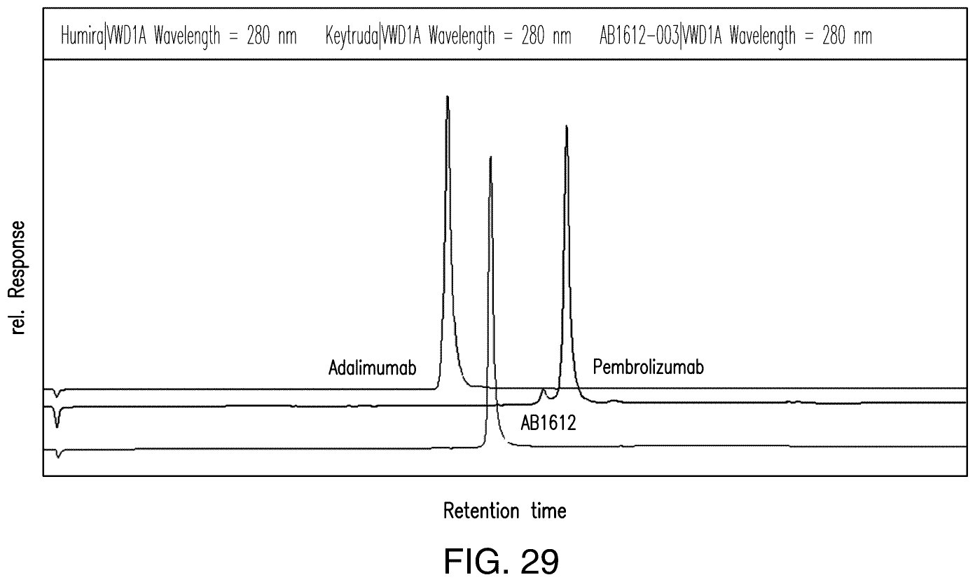

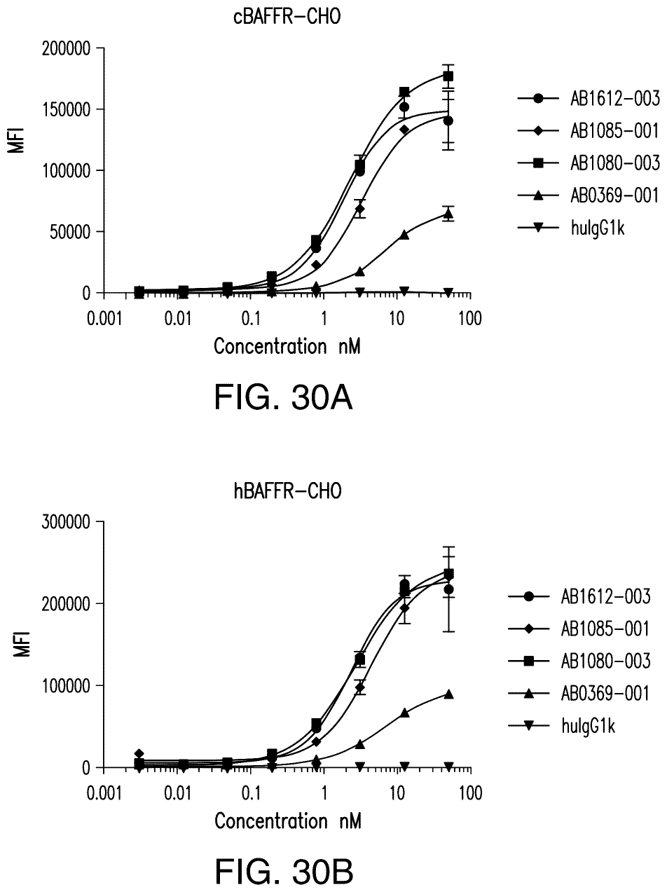

AB1080 and AB1081 showed improved binding to BAFF-R and did not contain any sequence liabilities in the CDR sequences; however, their hydrophobicity was high as compared to a panel of benchmarked therapeutic antibodies. AB1085 demonstrated desired hydrophobicity and affinity, but contained potential sequence liabilities in the CDRH2 and CDRH3 sequences (FIG. 28). Sequences of AB1080, AB1081 and AB1085 were compared and the AB1080 sequence was analyzed and further corrected, with a hydrophobicity reducing mutation W to Q generated (CDRH3: RFTMLRGWYIEDYGMDV (SEQ ID NO:14) to RFTMLRGQYIEDYGMDV (SEQ ID NO:13)). The resulting AB1424/AB1612 multispecific binding protein demonstrated favorable low hydrophobicity that falls within the range of well-behaved biologics (FIG. 29) while maintaining the same high affinity for BAFF-R (Table 13, FIG. 30A-30B), potent BAFF-R-BAFF binding blocking (FIG. 1) and comprising a liability free sequence that is characteristic for parental AB1080 (Table 14).

TABLE 13
Summary of BAFF-R binding and BAFF-R-BAFF blocking by
multispecific binding proteins AB1424/AB1612 lineage.

| | Cyno BAFF-R | Human BAFF-R | Ligand |
|---|---|---|---|
| | Cell Binding | Cell Binding | Blocking |
| Molecule | EC₅₀ (nM) | EC₅₀ (nM) | IC50 (nM) |
| AB0369-001 | 7.11 | 6.80 | >1000 |
| AB1080-003 | 2.36 | 2.71 | 5.72 |
| AB1085-001 | 3.29 | 4.48 | 12.67 |
| AB1612-003 | 1.85 | 3.09 | 6.76 |
| Human IgG1k | N/A | N/A | N/A |

TABLE 14
Comparison of BAFF-R binding CDRs in AB1424/AB1612 and its ancestors

| TriNKETs | CDRH1 | CDRH2 | CDRH3 |
|---|---|---|---|
| AB0369 | GFTFSSY | WYDGSN | RFTMLRGLIIEDYGMDV |
| | (SEQ ID NO: 1) | (SEQ ID NO: 2) | (SEQ ID NO: 3) |
| AB1080 | GFTFSSY | WYDASN | RFTHLRGWYIEDYGLDV |
| | (SEQ ID NO: 1) | (SEQ ID NO: 23) | (SEQ ID NO: 27) |
| AB1085 | GFTFGSY | WYDGSN | RFTHLRGQYIEDYGMDV |
| | (SEQ ID NO: 33) | (SEQ ID NO: 2) | (SEQ ID NO: 34) |
| AB1424/AB1612 | GFTFSSY | WYDASN | RFTHLRGQYIEDYGLDV |
| | (SEQ ID NO: 1) | (SEQ ID NO: 23) | (SEQ ID NO: 38) |

In conclusion, two antibody discovery campaigns utilizing recombinant protein and DNA immunization were conducted. The first campaign identified four medium affinity BAFF-R-BAFF non-blocking antibodies. A single binder, AB0369scFv that was discovered from the second campaign displayed the ability to block BAFF-R-BAFF interactions. Extensive development of AB0396scFv by multiple rounds of affinity maturation, liability correction, and rational sequence design resulted in the binder AB1612/AB1424, which demonstrated desirable properties for a therapeutic candidate.

### INCORPORATION BY REFERENCE

Unless stated to the contrary, the entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

What is claimed is:

1. An antigen-binding site that binds BAFF-R, comprising:
a heavy chain variable domain (VH) comprising a complementarity-determining region 1 (CDR1) sequence comprising an amino acid sequence of SEQ ID NO:50, a complementarity-determining region 2 (CDR2) sequence comprising an amino acid sequence of SEQ ID NO:51, and a complementarity-determining region 3 (CDR3) sequence comprising an amino acid sequence of SEQ ID NO:52; and
a light chain variable domain (VL) comprising a CDR1 sequence comprising an amino acid sequence of SEQ ID NO:4, a CDR2 sequence comprising an amino acid sequence of SEQ ID NO:5, and a CDR3 sequence comprising an amino acid sequence of SEQ ID NO:49.

2. An antigen-binding site that binds BAFF-R, wherein:
(a) the VH comprises CDR1, CDR2, and CDR3 sequences identical to the amino acid sequences of SEQ ID NOs:46, 47, and 48, respectively; and the VL comprises CDR1, CDR2, and CDR3 sequences identical to the amino acid sequences of SEQ ID NOs:4, 5, and 49, respectively;
(b) the VH comprises CDR1, CDR2, and CDR3 sequences identical to the amino acid sequences of SEQ ID NOs: 1, 2, and 16, respectively; and the VL comprises sequences identical to the amino acid sequences of SEQ ID NOs:4, 5, and 6, respectively;
(c) the VH comprises CDR1, CDR2, and CDR3 sequences identical to the amino acid sequences of SEQ ID NOs:21, 2, and 22, respectively; and the VL comprises CDR1, CDR2, and CDR3 sequences identical to the amino acid sequences of SEQ ID NOs:4, 5, and 6, respectively;
(d) the VH comprises CDR1, CDR2, and CDR3 sequences identical to the amino acid sequences of SEQ ID NOs:20, 23, and 26, respectively; and the VL comprises CDR1, CDR2, and CDR3 sequences identical to the amino acid sequences of SEQ ID NOs: 4, 5, and 6, respectively; or
(e) the VH comprises CDR1, CDR2, and CDR3 sequences identical to the amino acid sequences of SEQ ID NOs:35, 36, and 37, respectively; and the VL comprises CDR1, CDR2, and CDR3 sequences identical to the amino acid sequences of SEQ ID NOs:4, 5, and 49, respectively.

3. The antigen-binding site of claim 1 or 2, wherein the VH comprises CDR1, CDR2, and CDR3 sequences identical to the amino acid sequences of SEQ ID NOs:46, 47, and 48, respectively; and the VL comprises CDR1, CDR2, and CDR3 sequences identical to the amino acid sequences of SEQ ID NOs:4, 5, and 49, respectively.

4. The antigen-binding site of claim 1 or 2, wherein the VH comprises CDR1, CDR2, and CDR3 sequences identical to the amino acid sequences of SEQ ID NOs: 1, 23, and 38, respectively; and the VL comprises CDR1, CDR2, and CDR3 sequences identical to the amino acid sequences of SEQ ID NOs: 4, 5, and 39, respectively.

5. The antigen-binding site of claim 4, wherein the VH comprises an amino acid sequence at least 90%, at least 91%, at least 92%, at least 93%, at least 94%, at least 95%, at least 96%, at least 97%, at least 98%, or at least 99% identical to SEQ ID NO:40.

6. The antigen-binding site of claim 5, wherein the VH comprises a G44C substitution relative to SEQ ID NO:40.

7. The antigen-binding site of claim 5, wherein the VH comprises the amino acid sequence of SEQ ID NO:40.

8. The antigen-binding site of claim 5 or 6, wherein the VH comprises the amino acid sequence of SEQ ID NO:42.

9. The antigen-binding site of any one of claims 4-8, wherein the VL comprises an amino acid sequence at least 90%, at least 91%, at least 92%, at least 93%, at least 94%, at least 95%, at least 96%, at least 97%, at least 98%, or at least 99% identical to SEQ ID NO:41.

10. The antigen-binding site of claim 9, wherein the VL comprises a G100C substitution relative to SEQ ID NO:41.

11. The antigen-binding site of any one of claims 4-9, wherein the VL comprises the amino acid sequence of SEQ ID NO:41.

12. The antigen-binding site of any one of claims 4-10, wherein the VL comprises the amino acid sequence of SEQ ID NO:43.

13. An antigen-binding site comprising a VH comprising the amino acid sequence of SEQ ID NO:40 and a VL comprising the amino acid sequence of SEQ ID NO:41, or a VH comprising the amino acid sequence of SEQ ID NO:42 and a VL comprising the amino acid sequence of SEQ ID NO:43.

14. The antigen-binding site of any one of claims 1-13, wherein the antigen-binding site is present as a single-chain fragment variable (scFv), a Fab fragment, or a monoclonal antibody.

15. The antigen-binding site of any one of claims 1-14, wherein the antigen-binding site is present as a single-chain fragment variable (scFv).

16. The antigen-binding site of any one of claim 1-5, or 9, wherein the antigen-binding site is present as an scFv comprising an amino acid sequence at least 90% identical to the sequence of SEQ ID NO:44 or SEQ ID NO:45.

17. The antigen-binding site of any one of claims 14-16, wherein the scFv comprises an amino acid sequence identical to the sequence of SEQ ID NO:44 or SEQ ID NO:45.

18. The antigen-binding site of any one of claims 14-17, wherein the scFv comprises an amino acid sequence identical to the sequence of SEQ ID NO:44.

19. An antigen-binding site that competes with the antigen-binding site of any one of claims 1-18 for binding to BAFF-R.

20. The antigen-binding site of any one of claims 1-19, wherein the antigen-binding site binds human BAFF-R with a dissociation constant (K_{D}) smaller than or equal to 5 nM, as measured by surface plasmon resonance (SPR).

21. The antigen-binding site of any one of claims 1-20, wherein the antigen-binding site inhibits binding of BAFF-R to BAFF.

22. A protein comprising the antigen-binding site of any one of the claims 1-21.

23. The protein of claim 22, further comprising an antibody heavy chain constant region.

24. The protein of claim 23, wherein the antibody heavy chain constant region is a human IgG heavy chain constant region.

25. The protein of claim 24, wherein the antibody heavy chain constant region is a human IgG1 heavy chain constant region.

26. The protein of claim 24 or 25, wherein each polypeptide chain of the antibody heavy chain constant region comprises an amino acid sequence at least 90% identical to the amino acid sequence of wild-type human IgG1 Fc region.

27. The protein of any one of claims 24-26, wherein at least one polypeptide chain of the antibody heavy chain constant region comprises one or more mutations, relative to the amino acid sequence of wild-type human IgG1 Fc region, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system.

28. The protein of any one of claims 24-27, wherein at least one polypeptide chain of the antibody heavy chain constant region comprises one or more mutations, relative to the amino acid sequence of wild-type human IgG1 Fc region, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y4071, Y407V, K409F, K409W, K409D, T411D, T411E, K439D, and K439E, numbered according to the EU numbering system.

29. The protein of any one of claims 24-28, wherein one polypeptide chain of the antibody heavy chain constant region comprises one or more mutations, relative to the amino acid sequence of wild-type human IgG1 Fc region, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to the amino acid sequence of wild-type human IgG1 Fc region, at one or more positions selected from Q347, Y349, L351, S354, E356, E357, S364, T366, L368, K370, N390, K392, T394, D399, D401, F405, Y407, K409, T411, and K439, numbered according to the EU numbering system.

30. The protein of claim 29, wherein one polypeptide chain of the antibody heavy chain constant region comprises K360E and K409W substitutions relative to the amino acid sequence of wild-type human IgG1 Fc region; and the other polypeptide chain of the antibody heavy chain constant region comprises Q347R, D399V and F405T substitutions relative to the amino acid sequence of wild-type human IgG1 Fc region, numbered according to the EU numbering system.

31. The protein of claim 29 or 30, wherein one polypeptide chain of the antibody heavy chain constant region comprises a Y349C substitution relative to the amino acid sequence of wild-type human IgG1 Fc region; and the other polypeptide chain of the antibody heavy chain constant region comprises an S354C substitution relative to the amino acid sequence of wild-type human IgG1 Fc region, numbered according to the EU numbering system.

32. An antibody-drug conjugate comprising the protein of any one of claims 22-31 and a drug moiety.

33. The antibody-drug conjugate of claim 32, wherein the drug moiety is selected from the group consisting of auristatin, N-acetyl-γ calicheamicin, maytansinoid, pyrrolobenzodiazepine, and SN-38.

34. An immunocytokine comprising the antigen-binding site of any one of claims 1-21 and a cytokine.

35. The immunocytokine of claim 34, wherein the cytokine is selected from the group consisting of IL-2, IL-4, IL-10, IL-12, IL-15, TNF, and IFNα.

36. A bispecific T-cell engager comprising the antigen-binding site of any one of claims 1-21 and an antigen-binding site that binds CD3.

37. A chimeric antigen receptor (CAR) comprising:
(a) the antigen-binding site of any one of claims 1-21;
(b) a transmembrane domain; and
(c) an intracellular signaling domain.

38. The CAR of claim 37, wherein the transmembrane domain is selected from the transmembrane regions of the alpha, beta or zeta chain of the T-cell receptor, CD28, CD3 epsilon, CD45, CD4, CD5, CD8, CD9, CD16, CD22, BAFF-R, CD37, CD64, CD80, CD86, CD134, CD137, CD152, and CD154.

39. The CAR of claim 37 or 38, wherein the intracellular signaling domain comprises a primary signaling domain comprising a functional signaling domain of CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12.

40. The CAR of any one of claims 37-39, wherein the intracellular signaling domain further comprises a costimulatory signaling domain comprising a functional signaling domain of a costimulatory receptor.

41. The CAR of claim 40, wherein the costimulatory receptor is selected from the group consisting of OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

42. An isolated nucleic acid encoding the CAR of any one of claims 37-41.

43. An expression vector comprising the isolated nucleic acid of claim 42.

44. An immune effector cell comprising the nucleic acid of claim 42 or the expression vector of claim 43.

45. An immune effector cell expressing the CAR of any one of claims 37-41.

46. The immune effector cell of claim 44 or 45, wherein the immune effector cell is a T cell.

47. The immune effector cell of claim 46, wherein the T cell is a CD8⁺ T cell, a CD4⁺ T cell, a γδ T cell, or an NKT cell.

48. The immune effector cell of claim 44 or 45, wherein the immune effector cell is an NK cell.

49. A pharmaceutical composition comprising the protein of any one of claims 22-31, the antibody-drug conjugate of claim 32 or 33, the immunocytokine of claim 34 or 35, the bispecific T-cell engager of claim 36, or the immune effector cell of any one of claims 44-48; and a pharmaceutically acceptable carrier.

50. A method of treating cancer, the method comprising administering to a subject in need thereof an effective amount of the protein of any one of claims 22-31, the antibody-drug conjugate of claim 32 or 33, the immunocytokine of claim 34 or 35, the bispecific T-cell engager of claim 36, the immune effector cell of any one of claims 44-48, or the pharmaceutical composition of claim 49.

51. The method of claim 50, wherein the cancer is B-cell non-Hodgkin's lymphoma (B-NHL), chronic lymphocytic leukemia (CLL), mantle cell lymphoma (MCL), follicular lymphoma (FL), diffuse large B-cell lymphoma (DLBCL), marginal zone lymphoma, mucosa-associated lymphoid tissue (MALT) lymphoma, primary mediastinal B-cell lymphoma, and acute lymphocytic leukemia (ALL).

52. The method of claim 50 or 51, wherein the cancer expresses BAFF-R.

53. A method of treating an autoimmune inflammatory disease, the method comprising administering to a subject in need thereof an effective amount of the protein of any one of claims 22-31, the antibody-drug conjugate of claim 32 or 33, the immunocytokine of claim 34 or 35, the bispecific T-cell engager of claim 36, the immune effector cell of any one of claims 44-48, or the pharmaceutical composition of claim 49.

54. The antigen-binding site of any one of claims 1-21, the protein of any one of claims 22-31, the antibody-drug conjugate of claim 32 or 33, the immunocytokine of claim 34 or 35, or the bispecific T cell engager of claim 36, wherein the antigen-binding site, protein, antibody-drug conjugate, immunocytokine, or bispecific T cell engager is a purified antigen-binding site, protein, antibody-drug conjugate, immunocytokine, or bispecific T cell engager.

55. The antigen-binding site, protein, antibody-drug conjugate, immunocytokine, or bispecific T cell engager of claim 54, wherein the antigen-binding site, protein, antibody-drug conjugate, immunocytokine, or bispecific T cell engager is purified by a method selected from the group consisting of: centrifugation, depth filtration, cell lysis, homogenization, freeze-thawing, affinity purification, gel filtration, ion exchange chromatography, hydrophobic interaction exchange chromatography, and mixed-mode chromatography.


SEQUENCE LISTING (114 sequences)

SEQ ID NO: 1 - protein, 7 aa, synthetic construct
GFTFSSY

SEQ ID NO: 2 - protein, 6 aa, synthetic construct
WYDGSN

SEQ ID NO: 3 - protein, 17 aa, synthetic construct
RFTMLRGLIIEDYGMDV

SEQ ID NO: 4 - protein, 11 aa, synthetic construct
RASQSISSYLN

SEQ ID NO: 5 - protein, 7 aa, synthetic construct
AASSLQS

SEQ ID NO: 6 - protein, 9 aa, synthetic construct
QQSYSTPLT

SEQ ID NO: 7 - protein, 17 aa, synthetic construct
RFTMLRGVFIEDYGMDV

SEQ ID NO: 8 - protein, 11 aa, synthetic construct
RASQSVSSNLA

SEQ ID NO: 9 - protein, 7 aa, synthetic construct
GASTRAT

SEQ ID NO: 10 - protein, 7 aa, synthetic construct
GFTFSTY

SEQ ID NO: 11 - protein, 17 aa, synthetic construct
RNTMVRGVIIEDYGMDV

SEQ ID NO: 12 - protein, 9 aa, synthetic construct
QQSYSSPLT

SEQ ID NO: 13 - protein, 17 aa, synthetic construct
RFTMLRGQYIEDYGMDV

SEQ ID NO: 14 - protein, 17 aa, synthetic construct
RFTMLRGWYIEDYGMDV

SEQ ID NO: 15 - protein, 17 aa, synthetic construct
RFTMLRGWIIEDYGMDV

SEQ ID NO: 16 - protein, 17 aa, synthetic construct
  VARIANT 8: note = X is Q or W
  VARIANT 9: note = X is I or Y
RFTMLRGXXIEDYGMDV

SEQ ID NO: 17 - protein, 7 aa, synthetic construct
GFPFSSY

SEQ ID NO: 18 - protein, 7 aa, synthetic construct
GEWFSSY

SEQ ID NO: 19 - protein, 17 aa, synthetic construct
RFTHLRGWIIEDYGMDV

SEQ ID NO: 20 - protein, 7 aa, synthetic construct
GFTFSSS

SEQ ID NO: 21 - protein, 7 aa, synthetic construct
  VARIANT 2: note = X is F or E
  VARIANT 3: note = X is T, P, or W
  VARIANT 7: note = X is Y or S
GXXFSSX

SEQ ID NO: 22 - protein, 17 aa, synthetic construct
  VARIANT 4: note = X is M or H
  VARIANT 9: note = X is I or Y
RFTXLRGWXIEDYGMDV

SEQ ID NO: 23 - protein, 6 aa, synthetic construct
WYDASN

SEQ ID NO: 24 - protein, 17 aa, synthetic construct
RFTYLRGWYIEDYGLDV

SEQ ID NO: 25 - protein, 17 aa, synthetic construct
RFTSLRGWYIEDYGLDV

SEQ ID NO: 26 - protein, 17 aa, synthetic construct
  VARIANT 4: note = X is R, Y, or S
RFTXLRGWYIEDYGLDV

SEQ ID NO: 27 - protein, 17 aa, synthetic construct
RFTHLRGWYIEDYGLDV

SEQ ID NO: 28 - protein, 7 aa, synthetic construct
GFAFSSY

SEQ ID NO: 29 - protein, 6 aa, synthetic construct
WYDESN

SEQ ID NO: 30 - protein, 17 aa, synthetic construct
RFTNLRGWIIEDYGLDV

SEQ ID NO: 31 - protein, 7 aa, synthetic construct
GFTFSMY

SEQ ID NO: 32 - protein, 17 aa, synthetic construct
RFTRLRGWYIEDYGLDV

SEQ ID NO: 33 - protein, 7 aa, synthetic construct
GFTFGSY

SEQ ID NO: 34 - protein, 17 aa, synthetic construct
RFTHLRGQYIEDYGMDV

SEQ ID NO: 35 - protein, 7 aa, synthetic construct
  VARIANT 3: note = X is T or A
  VARIANT 5: note = X is S or G
  VARIANT 6: note = X is S or M
GFXFXXY

SEQ ID NO: 36 - protein, 6 aa, synthetic construct
  VARIANT 4: note = X is G, A, or E
WYDXSN

SEQ ID NO: 37 - protein, 17 aa, synthetic construct
  VARIANT 4: note = X is H, N, or R
  VARIANT 8: note = X is W or Q
  VARIANT 9: note = X is I or Y
  VARIANT 15: note = X is M or L
RFTXLRGXXIEDYGXDV

SEQ ID NO: 38 - protein, 17 aa, synthetic construct
RFTHLRGQYIEDYGLDV

SEQ ID NO: 39 - protein, 9 aa, synthetic construct
QQSYSIPLT

SEQ ID NO: 40 - protein, 126 aa, synthetic construct
EVQLVQSGGGVVQPGRSLRLSCAASGFTFSSYGMHWVRQAPGKGLEWVAVIWYDASNKYYGDSVKGRFTISRDNSKNTLYLQMNSLRDEDTAVYYCARRFTHLRGQYIEDYGLDVWGQGTTVTVSS

SEQ ID NO: 41 - protein, 107 aa, synthetic construct
EIVLTQSPSSLSASVGDRVTITCRASQSISSYLNWYQQKPGKAPKLLIYAASSLQSGVPSRFSGSGSGTDFTLTISSLQPEDFATYYCQQSYSIPLTFGGGTKVEIK

SEQ ID NO: 42 - protein, 126 aa, synthetic construct
EVQLVQSGGGVVQPGRSLRLSCAASGFTFSSYGMHWVRQAPGKCLEWVAVIWYDASNKYYGDSVKGRFTISRDNSKNTLYLQMNSLRDEDTAVYYCARRFTHLRGQYIEDYGLDVWGQGTTVTVSS

SEQ ID NO: 43 - protein, 107 aa, synthetic construct
EIVLTQSPSSLSASVGDRVTITCRASQSISSYLNWYQQKPGKAPKLLIYAASSLQSGVPSRFSGSGSGTDFTLTISSLQPEDFATYYCQQSYSIPLTFGCGTKVEIK

SEQ ID NO: 44 - protein, 253 aa, synthetic construct
EVQLVQSGGGVVQPGRSLRLSCAASGFTFSSYGMHWVRQAPGKCLEWVAVIWYDASNKYYGDSVKGRFTISRDNSKNTLYLQMNSLRDEDTAVYYCARRFTHLRGQYIEDYGLDVWGQGTTVTVSSGGGGSGGGGSGGGGSGGGGSEIVLTQSPSSLSASVGDRVTITCRASQSISSYLNWYQQKPGKAPKLLIYAASSLQSGVPSRFSGSGSGTDFTLTISSLQPEDFATYYCQQSYSIPLTFGCGTKVEIK

SEQ ID NO: 45 - protein, 253 aa, synthetic construct
EIVLTQSPSSLSASVGDRVTITCRASQSISSYLNWYQQKPGKAPKLLIYAASSLQSGVPSRFSGSGSGTDFTLTISSLQPEDFATYYCQQSYSIPLTFGCGTKVEIKGGGGSGGGGSGGGGSGGGGSEVQLVQSGGGVVQPGRSLRLSCAASGFTFSSYGMHWVRQAPGKCLEWVAVIWYDASNKYYGDSVKGRFTISRDNSKNTLYLQMNSLRDEDTAVYYCARRFTHLRGQYIEDYGLDVWGQGTTVTVSS

SEQ ID NO: 46 - protein, 7 aa, synthetic construct
  VARIANT 5: note = X is S or G
GFTFXSY

SEQ ID NO: 47 - protein, 6 aa, synthetic construct
  VARIANT 4: note = X is G or A
WYDXSN

SEQ ID NO: 48 - protein, 17 aa, synthetic construct
  VARIANT 4: note = X is M or H
  VARIANT 8: note = X is L, W, or Q
  VARIANT 9: note = X is I or Y
  VARIANT 15: note = X is M or L
RFTXLRGXXIEDYGXDV

SEQ ID NO: 49 - protein, 9 aa, synthetic construct
  VARIANT 6: note = X is T or I
QQSYSXPLT

SEQ ID NO: 50 - skipped

SEQ ID NO: 51 - protein, 6 aa, synthetic construct
  VARIANT 4: note = X is G, A, or E
WYDXSN

SEQ ID NO: 52 - protein, 17 aa, synthetic construct
  VARIANT 4: note = X is M, H, N, R, Y, or S
  VARIANT 8: note = X is L, Q, or W
  VARIANT 9: note = X is I or Y
  VARIANT 15: note = X is M or L
RFTXLRGXXIEDYGXDV

SEQ ID NO: 53 - protein, 9 aa, synthetic construct
FQGTHVPWT

SEQ ID NO: 54 - protein, 121 aa, synthetic construct
QVQLQQPGAELVKPGASVKLSCKASGYTFTSYWMHWVKQRPGQGLEWIGEIDPFDSYTNYNQNFKGKATLTVDKSSSTAYMLLSSLTSDDSAVYYCARERLRLWSYYFDYWGQGTTLTVSS

SEQ ID NO: 55 - protein, 112 aa, synthetic construct
DVVMTQTPLSLPVSLGDQASISCRSSQSIVHSNGNTYLEWYLQKPGQSPKLLIYKVSNRLSGVPDRFSGSGSGTDFTLKISRVEAEDLGVYYCFQGSHDPFTFGSGTKLEIK

SEQ ID NO: 56 - protein, 121 aa, synthetic construct
QVQLQQPGAELVKPGASVKLSCKASGYTFTSYWMHWVKQRPGQGLEWIGEVDPSDSYTNYNQKFKGKATLTVDKSSSTAYILLSNLTSDDSAVYYCARERVRLWSYFFDYWGQGTTLTVSS

SEQ ID NO: 57 - protein, 112 aa, synthetic construct; identical to SEQ ID NO: 55

SEQ ID NO: 58 - protein, 124 aa, synthetic construct
EVQLVESGGGLVKPGGSLKLSCVVSGFTFSNYAMSWVRQTPEKRLEWVATISDGGGYTYYPDSVKGRFTISRDNAKNNLYLQMSHLKSEDTAIYYCARDDLGGGNYVSSYFDVWGTGTTVTVSS

SEQ ID NO: 59 - protein, 112 aa, synthetic construct
DVVMTQTPLSLPVSPGDQASISCRSSQSLVHSNGNTYLYWYLQKPGQSPKLLIYRVSNRFSGVPDRFSGSGSGTDFTLKINRVEAEDLGVYFCFQGTHVPLTFGSGTKLELK

SEQ ID NO: 60 - protein, 125 aa, synthetic construct
EVQLVESGGGLVKPGGSLKLSCAASGFSFSRYAMSWVRQTPEKRLEWVATISDGGSYTHYRDNVKGRFTISRDNAKNNLNLQMSHLKSEDTAIYYCARNEMGLYFDYDVYAMDYWGQGTSVTVSS

SEQ ID NO: 61 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - skipped

SEQ ID NO: 63 - protein, 107 aa, synthetic construct
EIVLTQSPSSLSASVGDRVTITCRASQSISSYLNWYQQKPGKAPKLLIYAASSLQSGVPSRFSGSGSGTDFTLTISSLQPEDFATYYCQQSYSTPLTFGCGTKVEIK

SEQ ID NO: 64 - protein, 126 aa, synthetic construct
EVQLVQSGGGVVQPGRSLRLSCAASGFTFSSYGMHWVRQAPGKCLEWVAVIWYDGSNKYYGDSVKGRFTISRDNSKNTLYLQMNSLRDEDTAVYYCARRFTMLRGQYIEDYGMDVWGQGTTVTVSS

SEQ ID NO: 65 - protein, 126 aa, synthetic construct
EVQLVQSGGGVVQPGRSLRLSCAASGFTFSSYGMHWVRQAPGKCLEWVAVIWYDGSNKYYGDSVKGRFTISRDNSKNTLYLQMNSLRDEDTAVYYCARRFTMLRGWYIEDYGMDVWGQGTTVTVSS

SEQ ID NO: 66 - protein, 126 aa, synthetic construct
EVQLVQSGGGVVQPGRSLRLSCAASGFTFSSYGMHWVRQAPGKCLEWVAVIWYDGSNKYYGDSVKGRFTISRDNSKNTLYLQMNSLRDEDTAVYYCARRFTMLRGWIIEDYGMDVWGQGTTVTVSS

SEQ ID NO: 67 - protein, 126 aa, synthetic construct
EVQLVQSGGGVVQPGRSLRLSCAASGFPFSSYGMHWVRQAPGKCLEWVAVIWYDGSNKYYGDSVKGRFTISRDNSKNTLYLQMNSLRDEDTAVYYCARRFTMLRGWYIEDYGMDVWGQGTTVTVSS

SEQ ID NO: 68 - protein, 126 aa, synthetic construct
EVQLVQSGGGVVQPGRSLRLSCAASGEWFSSYGMHWVRQAPGKCLEWVAVIWYDGSNKYYGDSVKGRFTISRDNSKNTLYLQMNSLRDEDTAVYYCARRFTHLRGWIIEDYGMDVWGQGTTVTVSS

SEQ ID NO: 69 - protein, 126 aa, synthetic construct
EVQLVQSGGGVVQPGRSLRLSCAASGFTFSSSGMHWVRQAPGKCLEWVAVIWYDGSNKYYGDSVKGRFTISRDNSKNTLYLQMNSLRDEDTAVYYCARRFTMLRGWYIEDYGMDVWGQGTTVTVSS

SEQ ID NO: 70 - protein, 126 aa, synthetic construct
EVQLVQSGGGVVQPGRSLRLSCAASGFTFSSSGMHWVRQAPGKCLEWVAVIWYDASNKYYGDSVKGRFTISRDNSKNTLYLQMNSLRDEDTAVYYCARRFTRLRGWYIEDYGLDVWGQGTTVTVSS

SEQ ID NO: 71 - protein, 126 aa, synthetic construct
EVQLVQSGGGVVQPGRSLRLSCAASGFTFSSSGMHWVRQAPGKCLEWVAVIWYDASNKYYGDSVKGRFTISRDNSKNTLYLQMNSLRDEDTAVYYCARRFTYLRGWYIEDYGLDVWGQGTTVTVSS

SEQ ID NO: 72 - protein, 126 aa, synthetic construct
EVQLVQSGGGVVQPGRSLRLSCAASGFTFSSSGMHWVRQAPGKCLEWVAVIWYDASNKYYGDSVKGRFTISRDNSKNTLYLQMNSLRDEDTAVYYCARRFTSLRGWYIEDYGLDVWGQGTTVTVSS

SEQ ID NO: 73 - protein, 126 aa, synthetic construct
EVQLVQSGGGVVQPGRSLRLSCAASGFTFSSYGMHWVRQAPGKCLEWVAVIWYDASNKYYGDSVKGRFTISRDNSKNTLYLQMNSLRDEDTAVYYCARRFTHLRGWYIEDYGLDVWGQGTTVTVSS

SEQ ID NO: 74 - protein, 126 aa, synthetic construct
EVQLVQSGGGVVQPGRSLRLSCAASGFAFSSYGMHWVRQAPGKCLEWVAVIWYDESNKYYGDSVKGRFTISRDNSRNTLYLQMNSLRDEDTAVYYCARRFTNLRGWIIEDYGLDVWGQGTTVTVSS

SEQ ID NO: 75 - protein, 126 aa, synthetic construct
EVQLVQSGGGVVQPGRSLRLSCAASGFTFSMYGMHWVRQAPGKCLEWVAVIWYDASNKYYGDSVKGRFTISRDNSKNTLYLQMNSLRDEDTAVYYCARRFTRLRGWYIEDYGLDVWGQGTTVTVSS

SEQ ID NO: 76 - protein, 126 aa, synthetic construct
EVQLVQSGGGVVQPGRSLRLSCAASGFTFGSYGMHWVRQAPGKCLEWVAVIWYDGSNKYYGDSVKGRFTISRDNSKNTLYLQMNSLRDEDTAVYYCARRFTHLRGQYIEDYGMDVWGQGTTVTVSS

SEQ ID NO: 77 - protein, 126 aa, synthetic construct
EVQLVQSGGGVVQPGRSLRLSCAASGFTFSSYGMHWVRQAPGKCLEWVAVIWYDGSNKYYGDSVKGRFTISRDNSKNTLYLQMNSLRDEDTAVYYCARRFTMLRGLIIEDYGMDVWGQGTTVTVSS

SEQ ID NO: 78 - skipped

SEQ ID NO: 79 - protein, 112 aa, synthetic construct
DIVMTQTPLSLPVSLGDQASISCRSSQSLLHSNGNTYLYWYLQKPGQSPKLLIHRVSNRFSGVPDRFGGSGSGTDFTLKIIRVEAEDLGVYFCFQGTHVPWTFGGGTKLEIK

SEQ ID NO: 80 - protein, 7 aa, synthetic construct
GYTFTSY

SEQ ID NO: 81 - protein, 6 aa, synthetic construct
DPFDSY

SEQ ID NO: 82 - protein, 12 aa, synthetic construct
ERLRLWSYYFDY

SEQ ID NO: 83 - protein, 16 aa, synthetic construct
RSSQSIVHSNGNTYLE

SEQ ID NO: 84 - protein, 7 aa, synthetic construct
KVSNRLS

SEQ ID NO: 85 - protein, 9 aa, synthetic construct
FQGSHDPFT

SEQ ID NO: 86 - protein, 6 aa, synthetic construct
DPSDSY

SEQ ID NO: 87 - protein, 12 aa, synthetic construct
ERVRLWSYFFDY

SEQ ID NO: 88 - protein, 7 aa, synthetic construct
GFTFSNY

SEQ ID NO: 89 - protein, 5 aa, synthetic construct
DGGGY

SEQ ID NO: 90 - protein, 15 aa, synthetic construct
DDLGGGNYVSSYFDV

SEQ ID NO: 91 - protein, 16 aa, synthetic construct
RSSQSLVHSNGNTYLY

SEQ ID NO: 92 - protein, 7 aa, synthetic construct
RVSNRFS

SEQ ID NO: 93 - protein, 9 aa, synthetic construct
FQGTHVPLT

SEQ ID NO: 94 - protein, 7 aa, synthetic construct
GFSFSRY

SEQ ID NO: 95 - protein, 5 aa, synthetic construct
DGGSY

SEQ ID NO: 96 - protein, 16 aa, synthetic construct
NEMGLYFDYDVYAMDY

SEQ ID NO: 97 - protein, 16 aa, synthetic construct
RSSQSLLHSNGNTYLY

SEQ ID NO: 98 - protein, 20 aa, synthetic construct
GGGGSGGGGSGGGGSGGGGS

SEQ ID NO: 99 - protein, 20 aa, synthetic construct
GSGSGSGSGSGSGSGSGSGS

SEQ ID NO: 100 - protein, 30 aa, synthetic construct
GGSGGSGGSGGSGGSGGSGGSGGSGGSGGS

SEQ ID NO: 101 - protein, 40 aa, synthetic construct
GGGSGGGSGGGSGGGSGGGSGGGSGGGSGGGSGGGSGGGS

SEQ ID NO: 102 - protein, 40 aa, synthetic construct
GGSGGGSGGGSGGGSGGGSGGGSGGGSGGGSGGGSGGGSG

SEQ ID NO: 103 - protein, 50 aa, synthetic construct
GGSGGGGSGGGGSGGGGSGGGGSGGGGSGGGGSGGGGSGGGGSGGGGSGG

SEQ ID NO: 104 - protein, 50 aa, synthetic construct
GGGGSGGGGSGGGGSGGGGSGGGGSGGGGSGGGGSGGGGSGGGGSGGGGS

SEQ ID NO: 105 - protein, 15 aa, synthetic construct
GGGGSGGGGSGGGGS

SEQ ID NO: 106 - protein, 100 aa, synthetic construct
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 107 - protein, 100 aa, synthetic construct
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 108 - protein, 8 aa, synthetic construct
SGSGGGGS

SEQ ID NO: 109 - protein, 40 aa, synthetic construct
  source 1..40: note = up to 19 copies of GS could be deleted
GSGSGSGSGSGSGSGSGSGSGSGSGSGSGSGSGSGSGSGS

SEQ ID NO: 110 - protein, 60 aa, synthetic construct
  source 1..60: note = up to 19 copies of GGS could be deleted
GGSGGSGGSGGSGGSGGSGGSGGSGGSGGSGGSGGSGGSGGSGGSGGSGGSGGSGGSGGS

SEQ ID NO: 111 - protein, 4 aa, synthetic construct
  REPEAT 1..4: note = GGGS can be repeated n times, wherein n is 1, 2, 3, 4, 5, 6, 7, 8, 9, 10, 11, 12, 13, 14, 15, 16, 17, 18, 19, or 20
GGGS

SEQ ID NO: 112 - protein, 4 aa, synthetic construct
  REPEAT 1..4: note = GGSG can be repeated n times, wherein n is 1, 2, 3, 4, 5, 6, 7, 8, 9, 10, 11, 12, 13, 14, 15, 16, 17, 18, 19, or 20
GGSG

SEQ ID NO: 113 - protein, 5 aa, synthetic construct
  REPEAT 1..5: note = GGSGG can be repeated n times, wherein n is 1, 2, 3, 4, 5, 6, 7, 8, 9, 10, 11, 12, 13, 14, 15, 16, 17, 18, 19, or 20
GGSGG

SEQ ID NO: 114 - protein, 5 aa, synthetic construct
  REPEAT 1..5: note = GGGGS can be repeated n times, wherein n is 1, 2, 3, 4, 5, 6, 7, 8, 9, 10, 11, 12, 13, 14, 15, 16, 17, 18, 19, or 20
GGGGS